(19) United States
(12) Patent Application Publication
(10) Pub. No.: US 2023/0343414 A1
(43) Pub. Date: Oct. 26, 2023

(54) SEQUENCE-TO-SEQUENCE BASE CALLING

(71) Applicants: Illumina, Inc., San Diego, CA (US); Illumina Software, Inc., San Diego, CA (US)

(72) Inventors: Gery Vessere, Oakland, CA (US); Anindita Dutta, San Francisco, CA (US); Gavin Derek Parnaby, Laguna Niguel, CA (US)

(21) Appl. No.: 18/189,842

(22) Filed: Mar. 24, 2023

Related U.S. Application Data
(60) Provisional application No. 63/323,995, filed on Mar. 25, 2022.

(51) Int. Cl. G16B 40/00; G16B 30/00

(52) U.S. Cl. CPC G16B 30/00; G16B 40/00

(57) ABSTRACT

We disclose a computer-implemented method of base calling. The technology disclosed accesses a time series sequence of a read. Respective time series elements in the time series sequence represent respective bases in the read. Then, a composite sequence for the read is generated based on respective aggregate transformations of respective sliding windows of time series elements in the time series sequence. A subject composite element in the composite sequence is generated based on an aggregate transformation of a corresponding window of time series elements in the time series sequence. Then, the composite sequence is processed as an aggregate and generates a base call sequence that has respective base calls for the respective bases in the read.

### CROSS-REFERENCE TO RELATED APPLICATIONS

The present application claims the benefit of, and priority to, U.S. Provisional Application No. 63/323,995, entitled “SEQUENCE-TO-SEQUENCE BASE CALLING,” filed on Mar. 25, 2022. The aforementioned application is hereby incorporated by reference in its entirety.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep neural networks such as deep convolution neural networks for analyzing data.

### INCORPORATIONS

The following are incorporated by reference for all purposes as if fully set forth herein:
U.S. Provisional Pat. Application No.: 63/247,296, titled “STATE-BASED BASE CALLING PER-WELL STATE-BASED BASE CALLING,” filed 22 Sep. 2021 (Attorney Docket No. IP-2073-PRV);
U.S. Provisional Pat. Application No.: 63/247,301, titled “COMPRESSED STATE-BASED BASE CALLING SAMPLE SPACE-TO-PIXEL SPACE STATE TRANSFORMATION FOR BASE CALLING,” filed 22 Sep. 2021 (Attorney Docket No. IP-2208-PRV);
U.S. Nonprovisional Pat. Application No.: 17/308,035, titled “EQUALIZATION-BASED IMAGE PROCESSING AND SPATIAL CROSSTALK ATTENUATOR,” filed 4 May 2021 (Attorney Docket No. IP-1991-US);
U.S. Provisional Pat. Application No. 63/106,256, titled “SYSTEMS AND METHODS FOR PER-CLUSTER INTENSITY CORRECTION AND BASE CALLING,” filed 27 Oct. 2020 (Attorney Docket No. IP-2026-PRV);
U.S. Nonprovisional Pat. Application No. 15/909,437, titled “OPTICAL DISTORTION CORRECTION FOR IMAGED SAMPLES,” filed on 1 Mar. 2018;
U.S. Nonprovisional Pat. Application No. 16/825,987, titled “TRAINING DATA GENERATION FOR ARTIFICIAL INTELLIGENCE-BASED SEQUENCING,” filed 20 Mar. 2020 (Attorney Docket No. IP-1693-US);
U.S. Nonprovisional Pat. Application No. 16/825,991 titled “ARTIFICIAL INTELLIGENCE-BASED GENERATION OF SEQUENCING METADATA,” filed 20 Mar. 2020 (Attorney Docket No. IP-1741-US);
U.S. Nonprovisional Pat. Application No. 16/826,126, titled “ARTIFICIAL INTELLIGENCE-BASED BASE CALLING,” filed 20 Mar. 2020 (Attorney Docket No. IP-1744-US);
U.S. Nonprovisional Pat. Application No. 16/826,134, titled “ARTIFICIAL INTELLIGENCE-BASED QUALITY SCORING,” filed 20 Mar. 2020 (Attorney Docket No. IP-1747-US);
U.S. Nonprovisional Pat. Application No. 16/826,168, titled “ARTIFICIAL INTELLIGENCE-BASED SEQUENCING,” filed 21 Mar. 2020 (Attorney Docket No. IP-1752-US);
U.S. Nonprovisional Pat. Application No. 17/175,546, titled “ARTIFICIAL INTELLIGENCE-BASED BASE CALLING OF INDEX SEQUENCES,” filed 12 Feb. 2021 (Attorney Docket No. IP-1857-US);
U.S. Nonprovisional Pat. Application No. 17/180,542, titled “ARTIFICIAL INTELLIGENCE-BASED MANY-TO-MANY BASE CALLING,” filed 19 Feb. 2021 (Attorney Docket No. IP-1858-US);
U.S. Nonprovisional Pat. Application No. 17/176,151, titled “KNOWLEDGE DISTILLATION-BASED COMPRESSION OF ARTIFICIAL INTELLIGENCE-BASED BASE CALLER,” filed 15 Feb. 2021 (Attorney Docket No. IP-1859-US);
U.S. Provisional Pat. Application No. 63/072,032, titled “DETECTING AND FILTERING CLUSTERS BASED ON ARTIFICIAL INTELLIGENCE-PREDICTED BASE CALLS,” filed 28 Aug. 2020 (Attorney Docket No. IP-1860-PRV);
U.S. Provisional Pat. Application No. 63/161,880, titled “TILE LOCATION AND/OR CYCLE BASED WEIGHT SET SELECTION FOR BASE CALLING,” filed 16 Mar. 2021 (Attorney Docket No. IP-1861-PRV);
U.S. Provisional Pat. Application No. 63/161,896, titled “NEURAL NETWORK PARAMETER QUANTIZATION FOR BASE CALLING,” filed 16 Mar. 2021 (Attorney Docket No. IP-2049-PRV);
U.S. Nonprovisional Pat. Application No. 17/176,147, titled “HARDWARE EXECUTION AND ACCELERATION OF ARTIFICIAL INTELLIGENCE-BASED BASE CALLER,” filed 15 Feb. 2021 (Attorney Docket No. IP-1866-US);
U.S. Provisional Pat. Application No. 63/228,954, titled “BASE CALLING USING MULTIPLE BASE CALLER MODELS,” filed 3 Aug. 2021 (Attorney Docket No. IP-1856-PRV);
U.S. Nonprovisional Pat. Application No. 17/179,395, titled “DATA COMPRESSION FOR ARTIFICIAL INTELLIGENCE-BASED BASE CALLING,” filed 18 Feb. 2021 (Attorney Docket No. IP-1964-US);
U.S. Nonprovisional Pat. Application No. 17/180,480, titled “SPLIT ARCHITECTURE FOR ARTIFICIAL INTELLIGENCE-BASED BASE CALLER,” filed 19 Feb. 2021 (Attorney Docket No. IP-1982-US);
U.S. Nonprovisional Pat. Application No. 17/180,513, titled “BUS NETWORK FOR ARTIFICIAL INTELLIGENCE-BASED BASE CALLER,” filed 19 Feb. 2021 (Attorney Docket No. IP-1965-US);
U.S. Provisional Pat. Application No. 63/169,163, titled “ARTIFICIAL INTELLIGENCE-BASED BASE CALLER WITH CONTEXTUAL AWARENESS,” filed 31 Mar. 2021 (Attorney Docket No. IP-2007-PRV);
U.S. Provisional Pat. Application No. 63/216,419, titled “SELF-LEARNED BASE CALLER, TRAINED USING OLIGO SEQUENCES,” filed 29 Jun. 2021 (Attorney Docket No. IP-2050-PRV);
U.S. Provisional Pat. Application No. 63/216,404, titled “SELF-LEARNED BASE CALLER, TRAINED USING ORGANISM SEQUENCES,” filed 29 Jun. 2021 (Attorney Docket No. IP-2094-PRV);
U.S. Provisional Pat. Application No. 63/223,408, titled “SPECIALIST SIGNAL PROFILERS FOR BASE CALLING,” filed 19 Jul. 2021 (Attorney Docket No. IP-2063-PRV);
U.S. Provisional Pat. Application No. 63/226,707, titled “QUALITY SCORE CALIBRATION OF BASECALLING SYSTEMS,” filed 28 Jul. 2021 (Attorney Docket No. IP-2093-PRV);
U.S. Provisional Pat. Application No. 63/217,644, titled “EFFICIENT ARTIFICIAL INTELLIGENCE-BASED BASE CALLING OF INDEX SEQUENCES,” filed 1 Jul. 2021 (Attorney Docket No. IP-2135-PRV);
U.S. Nonprovisional Pat. Application No. 14/530,299, titled “IMAGE ANALYSIS USEFUL FOR PATTERNED OBJECTS,” filed on 31 Oct. 2014;
U.S. Nonprovisional Pat. Application No. 15/153,953, titled “METHODS AND SYSTEMS FOR ANALYZING IMAGE DATA,” filed on 3 Dec. 2014;
U.S. Nonprovisional Pat. Application No. 15/863,241, titled “PHASING CORRECTION,” filed on 5 Jan. 2018;
U.S. Nonprovisional Pat. Application No. 14/020,570, titled “CENTROID MARKERS FOR IMAGE ANALYSIS OF HIGH DENSITY CLUSTERS IN COMPLEX POLYNUCLEOTIDE SEQUENCING,” filed on 6 Sep. 2013;
U.S. Nonprovisional Pat. Application No. 12/565,341, titled “METHOD AND SYSTEM FOR DETERMINING THE ACCURACY OF DNA BASE IDENTIFICATIONS,” filed on 23 Sep. 2009;
U.S. Nonprovisional Pat. Application No. 12/295,337, titled “SYSTEMS AND DEVICES FOR SEQUENCE BY SYNTHESIS ANALYSIS,” filed on 30 Mar. 2007;
U.S. Nonprovisional Pat. Application No. 12/020,739, titled “IMAGE DATA EFFICIENT GENETIC SEQUENCING METHOD AND SYSTEM,” filed on 28 Jan. 2008;
U.S. Nonprovisional Pat. Application No. 13/833,619, titled “BIOSENSORS FOR BIOLOGICAL OR CHEMICAL ANALYSIS AND SYSTEMS AND METHODS FOR SAME,” filed on 15 Mar. 2013, (Attorney Docket No. IP-0626-US);
U.S. Nonprovisional Pat. Application No. 15/175,489, titled “BIOSENSORS FOR BIOLOGICAL OR CHEMICAL ANALYSIS AND METHODS OF MANUFACTURING THE SAME,” filed on 7 Jun. 2016, (Attorney Docket No. IP-0689-US);
U.S. Nonprovisional Pat. Application No. 13/882,088, titled “MICRODEVICES AND BIOSENSOR CARTRIDGES FOR BIOLOGICAL OR CHEMICAL ANALYSIS AND SYSTEMS AND METHODS FOR THE SAME,” filed on 26 Apr. 2013, (Attorney Docket No. IP-0462-US);
U.S. Nonprovisional Pat. Application No. 13/624,200, titled “METHODS AND COMPOSITIONS FOR NUCLEIC ACID SEQUENCING,” filed on 21 Sep. 2012, (Attorney Docket No. IP-0538-US);
U.S. Nonprovisional Pat. Application No. 13/006,206, titled “DATA PROCESSING SYSTEM AND METHODS,” filed on 13 Jan. 2011;
U.S. Nonprovisional Pat. Application No. 15/936,365, titled “DETECTION APPARATUS HAVING A MICROFLUOROMETER, A FLUIDIC SYSTEM, AND A FLOW CELL LATCH CLAMP MODULE,” filed on 26 Mar. 2018;
U.S. Nonprovisional Pat. Application No. 16/567,224, titled “FLOW CELLS AND METHODS RELATED TO SAME,” filed on 11 Sep. 2019;
U.S. Nonprovisional Pat. Application No. 16/439,635, titled “DEVICE FOR LUMINESCENT IMAGING,” filed on 12 Jun. 2019;
U.S. Nonprovisional Pat. Application No. 15/594,413, titled “INTEGRATED OPTOELECTRONIC READ HEAD AND FLUIDIC CARTRIDGE USEFUL FOR NUCLEIC ACID SEQUENCING,” filed on 12 May 2017;
U.S. Nonprovisional Pat. Application No. 16/351,193, titled “ILLUMINATION FOR FLUORESCENCE IMAGING USING OBJECTIVE LENS,” filed on 12 Mar. 2019;
U.S. Nonprovisional Pat. Application No. 12/638,770, titled “DYNAMIC AUTOFOCUS METHOD AND SYSTEM FOR ASSAY IMAGER,” filed on 15 Dec. 2009; and
U.S. Nonprovisional Pat. Application No. 13/783,043, titled “KINETIC EXCLUSION AMPLIFICATION OF NUCLEIC ACID LIBRARIES,” filed on 1 Mar. 2013.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

The rapid improvement in computation capability has made deep convolution neural networks (CNNs) a great success in recent years on many computer vision tasks with significantly improved accuracy. During the inference phase, many applications demand low latency processing of one image with strict power consumption requirement, which reduces the efficiency of graphics processing unit (GPU) and other general-purpose platform, bringing opportunities for specific acceleration hardware, e.g., field programmable gate array (FPGA), by customizing the digital circuit specific for the deep learning algorithm inference. However, deploying CNNs on portable and embedded systems is still challenging due to large data volume, intensive computation, varying algorithm structures, and frequent memory accesses.

As convolution contributes most operations in CNNs, the convolution acceleration scheme significantly affects the efficiency and performance of a hardware CNN accelerator. Convolution involves multiply and accumulate (MAC) operations with four levels of loops that slide along kernel and feature maps. The first loop level computes the MAC of pixels within a kernel window. The second loop level accumulates the sum of products of the MAC across different input feature maps. After finishing the first and second loop levels, a final output pixel is obtained by adding the bias. The third loop level slides the kernel window within an input feature map. The fourth loop level generates different output feature maps.

FPGAs have gained increasing interests and popularity in particular to accelerate the inference tasks, due to their (1) high degree of reconfigurability, (2) faster development time compared to application specific integrated circuits (ASICs) to catch up with the rapid evolving of CNNs, (3) good performance, and (4) superior energy efficiency compared to GPUs. The high performance and efficiency of an FPGA can be realized by synthesizing a circuit that is customized for a specific computation to directly process billions of operations with the customized memory systems. For instance, hundreds to thousands of digital signal processing (DSP) blocks on modern FPGAs support the core convolution operation, e.g., multiplication and addition, with high parallelism. Dedicated data buffers between external on-chip memory and on-chip processing engines (PEs) can be designed to realize the preferred dataflow by configuring tens of Mbyte on-chip block random access memories (BRAM) on the FPGA chip.

Efficient dataflow and hardware architecture of CNN acceleration are desired to minimize data communication while maximizing resource utilization to achieve high performance. An opportunity arises to design methodology and framework to accelerate the inference process of various CNN algorithms on acceleration hardware with high performance, efficiency, and flexibility.

The key feature of next generation sequencing (NGS) technologies is parallelization and the main mechanism underlying several sequencing platforms is sequencing-by-synthesis (SBS). Briefly, tens to hundreds of millions of random DNA fragments get sequenced simultaneously by sequentially building up complementary bases of single-stranded DNA templates and by capturing the synthesis information in a series of raw images of fluorescence.

Extracting the actual sequence information (i.e., strings in {A, C, G, T}) from image data involves two computational tasks, namely image analysis and base calling. The primary function of image analysis is to translate image data into fluorescence intensity data for each DNA fragment, while the goal of base calling is to infer sequence information from the obtained intensity data.

There are a number of stochastic and contextual sources of variation that can reduce base calling accuracy. For example, k-mer biases in base calling are affected by GC content of the sequenced genome. Base callers can exhibit bias when applied to GC-rich regions of DNA, primarily due to reduced sequence complexity but also as a result of polymerase chain reaction (PCR) bias during amplification steps.

The accuracy of base calling is of essential importance for various downstream applications including sequence assembly, SNP calling, and genotype calling. Improving base calling accuracy can enable achieving desired performance of downstream applications with smaller sequencing coverage, which translates to a reduction in the sequencing cost.

Training neural networks for base calling requires large amounts of computer memory, which increases exponentially with increasing image size and numerosity. Computer memory becomes a limiting factor because the backpropagation algorithm for optimizing deep neural networks requires the storage of intermediate activations. Since the size and numerosity of these intermediate activations increases proportionate to the input size and numerosity, memory quickly fills up with larger and more images.

Base callers that use neural networks, for example, the ones disclosed in commonly owned Patent Application Nos. 16/826,126; 16/826,134; 16/826,168; 17/175,546; 17/180,542; 17/176,151; 63/072,032; 63/161,880; 63/161,896; 17/176,147; 63/228,954; 17/179,395; 17/180,480; 17/180,513; 63/169,163; and 63/217,644, make a base call prediction using image data for a sliding window of sequencing cycles, according to one implementation. Increasing the size of the sliding window to include image data from more sequencing cycles would increase complexity of the neural networks and also add additional burden on available compute and memory.

An opportunity arises to configure base calling operations to incorporate contextual information from a multitude of past and future sequencing cycles. More accurate base calling with reduced error rates, particularly towards attenuating k-mer bias, may result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 illustrates one implementation of sequence-to-sequence base calling.

FIG. 2 is a schematic representation of an encoder-decoder architecture.

FIG. 3 shows an overview of an attention mechanism added onto an RNN encoder-decoder architecture.

FIG. 4 is a schematic representation of the calculation of self-attention showing one attention head.

FIG. 5 depicts several attention heads in a Transformer block.

FIG. 6 shows parallel execution of multi-head attention logics.

FIG. 7 portrays one encoder layer of a Transformer network.

FIG. 8 shows a schematic overview of a Transformer model.

FIG. 9A shows a Vision Transformer (ViT).

FIG. 9B shows a Transformer block used by the Vision Transformer.

FIGS. 10A, 10B, 10C, and 10D show details of the Transformer block of FIG. 9B.

FIG. 11 shows an example source code implementing the Vision Transformer.

FIG. 12 has a left plot that depicts base calling error rate measured on a ground truth dataset comparing RTA base caller (a non-neural network-based base caller) and the disclosed Transformer-based base caller with full read context, and a right plot, which is the same as the left plot but measures the fractional base calling error rate improvement across sequencing cycles by the disclosed Transformer-based base caller.

FIG. 13 illustrates a hyperparameter scan of the length of the k-mer used as the input to the disclosed Transformer-based base caller.

FIG. 14 represents the training loss across epochs through the dataset and that ~70 epochs are needed to train the disclosed Transformer-based base caller.

FIG. 15 depicts the learned feature maps that represent the positional and token embeddings used as the input of the disclosed Transformer-based base caller.

FIG. 16 describes the attention maps for a 2 Layers (rows), 4-Heads per Layer (columns) implementation of the disclosed Transformer-based base caller trained on full sequence context.

FIG. 17 shows Layer 2, Head 4 attention maps of the disclosed Transformer-based base caller from 3 different clusters that have the same sequence but are offset by a few sequencing cycles in the sequencing run.

FIG. 18 has plots that show the decision boundary differentials for a few cycles of a sequence if we force the disclosed Transformer-based base caller to only consider the center cycle in Layer 2, Head 4.

FIGS. 19 and 20 have plots that show the improvement in the base calling error rate of the disclosed Transformer-based base caller v/s the RTA base caller (a non-neural network-based base caller).

FIG. 21 shows the improvement obtained when training the disclosed Transformer-based base caller on a bacterial dataset and testing on a human dataset.

FIG. 22 shows where most of the gains of the disclosed Transformer-based base caller in base calling error rate come from in terms of counts of errors per read.

FIG. 23 shows the improvements measured in homopolymer over the whole testing dataset.

FIG. 24 figure shows one sequence with a large homopolymer that has a sequence-specific error profile.

FIG. 25 shows how the disclosed Transformer-based base caller adjusts its decision boundaries strongly based on the preceding 2 bases of context.

FIGS. 26A and 26B depict one implementation of a sequencing system that comprises a configurable processor.

FIG. 26C is a simplified block diagram of a system for analysis of sensor data from the sequencing system, such as base call sensor outputs.

FIG. 27A is a simplified diagram showing aspects of the base calling operation, including functions of a runtime program executed by a host processor.

FIG. 27B is a simplified diagram of a configuration of a configurable processor.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein can be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

### Introduction

The technology disclosed relates to using a Transformer model for base calling. In particular, the technology disclosed proposes a parallel input, parallel output (PIPO) base caller based on the Transformer architecture. The PIPO base caller processes, in parallel, a sequence of sequencing images for a sequence of sequencing/base calling cycles of a sequencing run, and produces, in parallel, a sequence of base calls for the sequence of sequencing/base calling cycles. The Transformer model relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input’s representation is also directly informed by all other inputs’ representations, this results in an effectively global receptive field across the whole input sequence. This stands in contrast to, e.g., convolutional architectures which typically only have a limited receptive field.

### Sequencing Images

Base calling is the process of determining the nucleotide composition of a sequence. In one implementation, base calling involves analyzing image data, i.e., sequencing images 102, produced during a sequencing run (or sequencing reaction) carried out by a sequencing system such as Illumina’s HiSeqX, HiSeq 3000, HiSeq 4000, HiSeq 2500, NovaSeq 6000, NextSeq 550, NextSeq 1000, NextSeq 2000, NextSeqDx, MiSeq, and MiSeqDx. In other implementations, base calling can involve inferring sequence reads from non-image sequencing data.

A sequencing system can be used for the sequencing of nucleic acids. Applicable techniques include those where nucleic acids are attached at fixed locations in an array (e.g., the wells of a flow cell) and the array is imaged repeatedly. In such implementations, the sequencing system can obtain images in two different color channels, which can be used to distinguish a particular nucleotide base type from another. The sequencing system can implement base calling-the process of determining a base (e.g., adenine (A), cytosine (C), guanine (G), or thymine (T)) for a given spot location of an image at an imaging cycle. During two-channel base calling, for example, image data extracted from two images can be used to determine the presence of one of four base types by encoding base identity as a combination of the intensities of the two images. For a given spot or location in each of the two images, base identity can be determined based on whether the combination of signal identities is [on, on], [on, off], [off, on], or [off, off].

Output data from the sequencing system can be communicated to a real-time analysis module (not shown), which can include the Transformer-based base caller 122. Real-time analysis module, in various implementations, executes computer readable instructions for analyzing the image data (e.g., image quality scoring, base calling, etc.), reporting or displaying the characteristics of the beam (e.g., focus, shape, intensity, power, brightness, position) to a graphical user interface (GUI), etc. These operations can be performed in real-time during imaging cycles to minimize downstream analysis time and provide real-time feedback and troubleshooting during an imaging run. In implementations, real-time analysis module can be a computing device that is communicatively coupled to and controls an imaging sub-system of the sequencing system.

The following discussion outlines how the sequencing images 102 are generated and what they depict, in accordance with one implementation.

In some implementations, base calling decodes the intensity data encoded in the sequencing images 102 into nucleotide sequences. In one implementation, the Illumina sequencing platforms employ cyclic reversible termination (CRT) chemistry for base calling. The process relies on growing nascent strands complementary to template strands with fluorescently-labeled nucleotides, while tracking the emitted signal of each newly added nucleotide. The fluorescently-labeled nucleotides have a 3′ removable block that anchors a fluorophore signal of the nucleotide type.

Sequencing occurs in repetitive cycles, each comprising three steps: (a) extension of a nascent strand by adding the fluorescently-labeled nucleotide; (b) excitation of the fluorophore using one or more lasers of an optical sub-system of the sequencing system and imaging through different filters of the optical sub-system, yielding the sequencing images 102; and (c) cleavage of the fluorophore and removal of the 3′ block in preparation for the next sequencing cycle. Incorporation and imaging cycles are repeated up to a designated number of sequencing cycles, defining the read length. Using this approach, each cycle interrogates a new position along the template strands.

The tremendous power of the Illumina sequencers stems from their ability to simultaneously execute and sense millions or even billions of clusters (also called “clusters”) undergoing CRT reactions. A cluster comprises approximately one thousand identical copies of a template strand, though clusters vary in size and shape. The clusters are grown from the template strand, prior to the sequencing run, by bridge amplification or exclusion amplification of the input library. The purpose of the amplification and cluster growth is to increase the intensity of the emitted signal since the imaging device cannot reliably sense the fluorophore signal of a single strand. However, the physical distance of the strands within a cluster is small, so the imaging device perceives the cluster of strands as a single spot.

Sequencing occurs in a flow cell (or biosensor) - a small glass slide that holds the input strands. The flow cell is connected to the optical system, which comprises microscopic imaging, excitation lasers, and fluorescence filters. The flow cell comprises multiple chambers called lanes. The lanes are physically separated from each other and may contain different tagged sequencing libraries, distinguishable without sample cross-contamination. In some implementations, the flow cell comprises a patterned surface. A “patterned surface” refers to an arrangement of different regions in or on an exposed layer of a solid support.

The imaging device of the sequencing system (e.g., a solid-state imager such as a charge-coupled device (CCD) or a complementary metal-oxide-semiconductor (CMOS) sensor) takes snapshots at multiple locations along the lanes in a series of non-overlapping regions called tiles. For example, there can be sixty-four or ninety-six tiles per lane. A tile holds hundreds of thousands to millions of clusters.

The output of the sequencing run is the sequencing images 102. Sequencing images 102 depict intensity emissions of the clusters and their surrounding background using a grid (or array) of pixelated units (e.g., pixels, superpixels, subpixels). The intensity emissions are stored as intensity values of the pixelated units. The sequencing images 102 have dimensions w × h of the grid of pixelated units, where w (width) and h (height) are any numbers ranging from 1 and 100,000 (e.g., 115 ×115, 200 × 200, 1800 × 2000, 2200 × 25000, 2800 × 3600, 4000 × 400). In some implementations, w and h are the same. In other implementations, w and h are different. The sequencing images 102 depict intensity emissions generated as a result of nucleotide incorporation in the nucleotide sequences during the sequencing run. The intensity emissions are from associated clusters and their surrounding background.

A data flow logic (not shown) provides the sequencing images 102 to the Transformer-based base caller 122 for base calling, in accordance with one implementation. The Transformer-based base caller 122 accesses the sequencing images 102 on a patch-by-patch basis (or a tile-by-tile basis), in accordance with one implementation. Each of the patches is a sub-grid (or sub-array) of pixelated units in the grid of pixelated units that forms the sequencing images 102. The patches have dimensions q × r of the sub-grid of pixelated units, where q (width) and r (height) are any numbers ranging from 1 and 10000 (e.g., 3 × 3, 5 × 5, 7 × 7, 10 × 10, 15 × 15, 25 × 25, 64 × 64, 78 × 78, 115 × 115). In some implementations, q and r are the same. In other implementations, q and r are different. In some implementations, the patches extracted from a sequencing image are of the same size. In other implementations, the patches are of different sizes. In some implementations, the patches can have overlapping pixelated units (e.g., on the edges).

Sequencing produces m sequencing images 102 per sequencing cycle for corresponding m image channels, in accordance with some implementations. That is, each of the sequencing images 102 has one or more image (or intensity) channels (analogous to the red, green, blue (RGB) channels of a color image). In one implementation, each image channel corresponds to one of a plurality of filter wavelength bands. In another implementation, each image channel corresponds to one of a plurality of imaging events at a sequencing cycle. In yet another implementation, each image channel corresponds to a combination of illumination with a specific laser and imaging through a specific optical filter. The image patches are tiled (or accessed) from each of the m image channels for a particular sequencing cycle. In different implementations such as 4-, 2-, and 1-channel chemistries, m is 4 or 2. In other implementations, m is 1, 3, or greater than 4. In other implementations, the images can be in blue and violet color channels instead of or in addition to the red and green color channels.

Consider, for example, that a sequencing run is implemented using two different image channels: a blue channel and a green channel. Then, at each sequencing cycle, the sequencing run produces a blue image and a green image. This way, for a series of k sequencing cycles of the sequencing run, a sequence of k pairs of blue and green images is produced as output and stored as the sequencing images 102. Accordingly, a sequence of k pairs of blue and green image patches is generated for the patch-level processing by the Transformer-based base caller 122.

### State Data

Current sequencing data includes sequencing data generated by the sequencing system for a current sequencing cycle of a sequencing run. The current sequencing cycle can be identified as the “N” sequencing cycle. Previous sequencing data includes sequencing data generated by the sequencing system for one or more previous sequencing cycles of the sequencing run. The previous sequencing cycles precede the current sequencing cycle. The previous sequencing cycles can be identified as the “1 to N-1” sequencing cycles. In other implementations, the previous sequencing data can include sequencing data for a subset of the 1 to N-1 sequencing cycles.

A state generator (not shown) uses the current sequencing data and the previous sequencing data to generate current state data for the current sequencing cycle. The state generator can be a value or function that is applied to the sequencing data to generate a desired result. The state generator can be applied to the sequencing data by any of a variety of mathematical manipulations including, but not limited to addition, subtraction, division, multiplication, or a combination thereof. The state generator can be a mathematical formula, logic function, computer implemented algorithm, or the like. The sequencing data can be image data, electrical data, or a combination thereof.

In one implementation, the state generator generates the current state data by accumulating summary statistics of the current sequencing data and the previous sequencing data. Examples of the summary statistics include maximum value, minimum value, average (mean), exponential weighted average, moving (running) average, exponential moving average, mode, standard deviation, variance, skewness, kurtosis, percentiles, and entropy. In other implementations, the state generator determines secondary statistics based on the summary statistics. Examples of the secondary statistics include deltas, sums, series of maximum values, series of minimum values, minimum of the maximum values in the series, and maximum of minimum values in the series.

The Transformer-based base caller 122 generates current base call data for the current sequencing cycle in response to processing the current sequencing data and the current state data. The current base call data can include base calls for one or more clusters. In some implementations, the current sequencing data and the current state data are combined prior to processing by the Transformer-based base caller 122. The combination can be brought about by, for example, summing operations, element-wise multiplication operations, element-wise multiplication and summation (convolution) operations, and concatenation operations.

### Intensity Readings

In some implementations, the Transformer-based base caller 122 (e.g., the neural network-based base caller) can take as input intensity readings. In one implementation, base calling involves analyzing intensity readings produced during a sequencing run (or sequencing reaction) carried out by a CMOS sensor-based sequencing system (biosensor) such as Illumina’s iSeq. Such a biosensor can include a flow cell that is mounted onto a sampling device. The sampling device can be similar to, for example, an integrated circuit comprising a plurality of stacked substrate layers. The substrate layers can include a base substrate, a solid-state imager (e.g., CMOS sensor), a filter or light-management layer, and a passivation layer. The sampling device can be manufactured using processes that are similar to those used in manufacturing integrated circuits. The base substrate and the solid-state imager can be provided together as a previously constructed solid-state imaging device (e.g., CMOS chip). For example, the base substrate can be a wafer of silicon and the solid-state imager can be mounted thereon. The solid-state imager can be manufactured as a single chip through a CMOS-based fabrication process. The solid-state imager can include a layer of semiconductor material (e.g., silicon) and an array of sensors. The sensors can be photodiodes configured to detect light. The sensors can comprise light detectors.

The array of sensors can be communicatively coupled to a row decoder and a column amplifier or decoder. The column amplifier can also be communicatively coupled to a column analog-to-digital converter (Column ADC/Mux). Circuitry formed within the sampling device can be configured for at least one of signal amplification, digitization, storage, and processing. The circuitry can collect and analyze the detected fluorescent light and generate pixel signals/detection signals/intensity readings and communicate them to a signal processor and memory. The circuitry can also perform additional analog and/or digital signal processing in the sampling device. The sampling device can include conductive vias that perform signal routing (e.g., transmit the intensity readings to the signal processor). The intensity readings can also be transmitted through electrical contacts of the sampling device.

The sampling device can also take other forms. For example, the sampling device can comprise a CCD device, such as a CCD camera, that is coupled to a flow cell or is moved to interface with a flow cell having reaction sites therein. The sampling device can be a CMOS-fabricated sensor, including chemically-sensitive field-effect transistors (chemFETs), ion-sensitive field-effect transistors (ISFET), and/or metal-oxide semiconductor field-effect transistors (MOSFET). The sampling device can include an array of field-effect transistors (FETs) that can be configured to detect a change in electrical properties within the reaction chambers. For example, the FETs can detect at least one of a presence and concentration change of various analytes. For example, the array of FETs can monitor changes in hydrogen ion concentration.

### Non-Intensity Readings

In some implementations, the Transformer-based base caller 122 (e.g., the neural network-based base caller) can take as input non-intensity sequencing data. The sequencing system can also generate non-intensity sequencing data, in accordance with other implementations. In one implementation, the sequencing data can be based on pH changes induced by the release of hydrogen ions during molecule extension. The pH changes can be detected and converted to a voltage change that is proportional to the number of bases incorporated. In yet another implementation, the sequencing data can be constructed, for example, from nanopore sensing that uses biosensors to measure the disruption in current as a cluster passes through a nanopore or near its aperture while determining the identity of the base. In one implementation, the nanopore-based sequencing can be based on the following concept: pass a single strand of DNA (or RNA) through a membrane via a nanopore and apply a voltage difference across the membrane. The nucleotides present in the pore can affect the pore’s electrical resistance, so current measurements over time can indicate the sequence of DNA bases passing through the pore. This electrical current signal (the ‘squiggle’ due to its appearance when plotted) is the raw data gathered by a sequencer. These measurements can be stored as 16-bit integer data acquisition (DAC) values, taken at 4 kHz frequency (for example). With a DNA strand velocity of ~450 base pairs per second, this can give approximately nine raw observations per base on average. This signal can then be processed to identify breaks in the open pore signal corresponding to individual reads. These stretches of the raw signal can be base called - the process of converting DAC values into a sequence of DNA bases. In some implementations, the sequencing data can comprise normalized or scaled DAC values.

### Base Caller

Examples of the Transformer-based base caller 122 include different base calling procedures available for the Illumina platform, such as Real-Time Analysis (RTA), BlindCall, freeIbis, Softy, AYB, OnlineCall, BM-BC, ParticleCall, TotalReCaller, naiveBayesCall, Srfim, BayesCall, Ibis, Rolexa, Alta-Cyclic, and Bustard. Examples of the Transformer-based base caller 122 also include Illumina’s neural network-based offerings, such as the ones disclosed in commonly owned Patent Application Nos. 16/825,987; 16/825,991; 16/826,126; 16/826,134; 16/826,134; 16/826,168; 17/175,546; 17/180,542; 17/176,151; 63/072,032; 63/161,880; 63/161,896; 17/176,147; 63/228,954; 17/179,395; 17/180,480; 17/180,513; 63/169,163; and 63/217,644, collectively referred to herein as “DeepRTA” or “Deep Leaming Primary Analysis.” Yet other examples of the Transformer-based base caller 122 include different base calling procedures available for the Oxford Nanopore Technologies (ONT), such as Metrichor, Nanocall, DeepNano, Nanonet, Scrappie, Albacore, Guppy, Basecrawller, Chiron, Halcyon, MinCall, SACall, Causalcall, and WaveNano.

### Neural Network-Based Base Caller

In some implementations, the Transformer-based base caller 122 is a neural network-based base caller. In one implementation, the neural network-based base caller processes the sequencing images 102 (or patches thereof) through its hidden layers and produces an alternative representation. The alternative representation is then used by an output layer (e.g., a softmax layer) for generating base calls, which form the sequencing reads.

In one implementation, the neural network-based base caller outputs a base call for a target cluster for each sequencing cycle in a plurality of sequencing cycles, thereby producing a base call sequence for the target cluster. In another implementation, the neural network-based base caller outputs a base call for each target cluster in a plurality of target clusters for each sequencing cycle in a plurality of sequencing cycles, thereby producing a base call sequence for each target cluster.

In one implementation, the neural network-based base caller is a multilayer perceptron (MLP). In another implementation, the neural network-based base caller is a feedforward neural network. In yet another implementation, the neural network-based base caller is a fully-connected neural network. In a further implementation, the neural network-based base caller is a fully convolution neural network. In yet further implementation, the neural network-based base caller is a semantic segmentation neural network. In yet another further implementation, the neural network-based base caller is a generative adversarial network (GAN) (e.g., CycleGAN, StyleGAN, pixelRNN, text-2-image, DiscoGAN, IsGAN).

In one implementation, the neural network-based base caller is a convolution neural network (CNN) with a plurality of convolution layers. In another implementation, the neural network-based base caller is a recurrent neural network (RNN) such as a long short-term memory network (LSTM), bi-directional LSTM (Bi-LSTM), or a gated recurrent unit (GRU). In yet another implementation, the neural network-based base caller includes both a CNN and an RNN.

In yet other implementations, the neural network-based base caller can use 1D convolutions, 2D convolutions, 3D convolutions, 4D convolutions, 5D convolutions, dilated or atrous convolutions, transpose convolutions, depthwise separable convolutions, pointwise convolutions, 1 × 1 convolutions, group convolutions, flattened convolutions, spatial and cross-channel convolutions, shuffled grouped convolutions, spatial separable convolutions, and deconvolutions. The neural network-based base caller can use one or more loss functions such as logistic regression/log loss, multi-class cross-entropy/softmax loss, binary cross-entropy loss, mean-squared error loss, L1 loss, L2 loss, smooth L1 loss, and Huber loss. The neural network-based base caller can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The neural network-based base caller can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential linear unit (ELU), sigmoid and hyperbolic tangent (tanh)), batch normalization layers, regularization layers, dropout, pooling layers (e.g., max or average pooling), global average pooling layers, and attention mechanisms (e.g., self-attention).

The Transformer-based base caller 122 can be a rule-based model, linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The Transformer-based base caller 122 can be an ensemble of multiple models, in some implementations.

The neural network-based base caller is trained using backpropagation-based gradient update techniques. Example gradient descent techniques that can be used for training the neural network-based base caller include stochastic gradient descent, batch gradient descent, and mini-batch gradient descent. Some examples of gradient descent optimization algorithms that can be used to train the neural network-based base caller are Momentum, Nesterov accelerated gradient, Adagrad, Adadelta, RMSprop, Adam, AdaMax, Nadam, and AMSGrad.

### Transformer-Based Base Caller

In different implementations, the neural network-based base caller includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN + FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

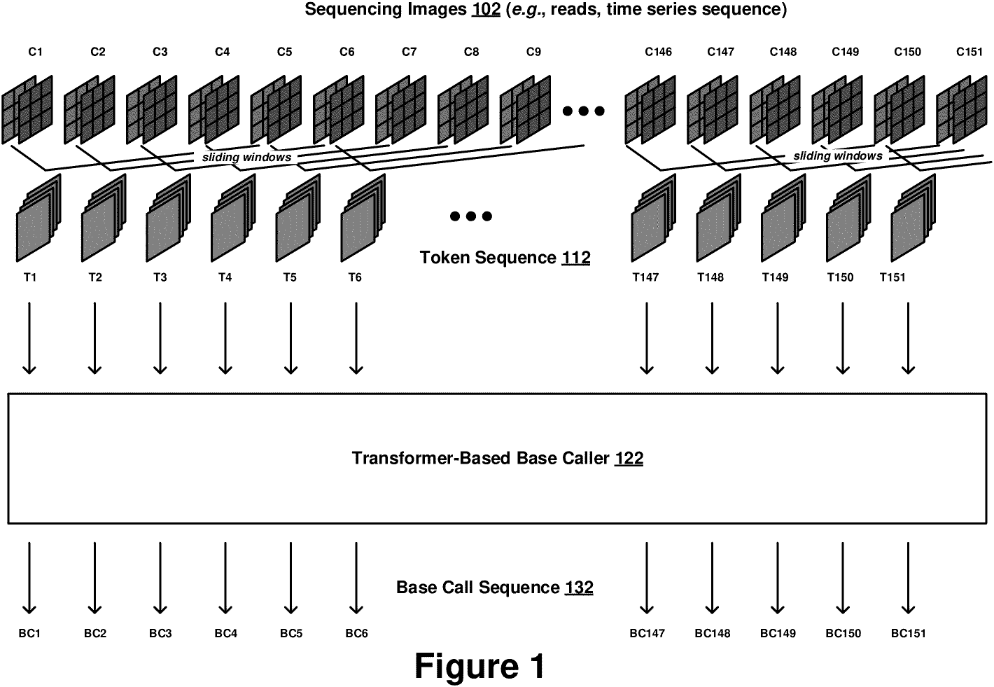

FIG. 1 illustrates one implementation of sequence-to-sequence base calling. The technology disclosed accesses a time series sequence of a read. In one implementation, the read is encoded in the sequencing images 102.

Respective time series elements in the time series sequence represent respective bases in the read. In one implementation, the respective time series elements are respective image sets for respective sequencing cycles (e.g., cycle 1-cycle 151 (c1-c151)) of a sequencing run. In such an implementation, each image has multiple color channels (e.g., a red color channel and a blue color channel).

In another implementation, the respective time series elements are respective intensity values (e.g., intensity readings/tracks) for respective sequencing cycles of a sequencing run. In such an implementation, each of the respective intensity values has respective channel-specific measurements for respective channels (e.g., a red intensity channel and a blue intensity channel). In some implementations, the respective intensity values are corrected for scale variation and shift variation.

In yet another implementation, the respective time series elements are respective voltage values for respective sequencing cycles of a sequencing run. In yet another implementation, the respective time series elements are respective current values for respective sequencing cycles of a sequencing run.

In some implementations, the respective time series elements are supplemented with respective state values for respective sequencing cycles of a sequencing run. In one implementation, the respective state values are channel-specific.

A composite sequence (e.g., a token sequence 112) for the read is generated based on respective aggregate transformations of time series elements in the time series sequence. As used herein, the phrase “aggregate transformation” refers to analyzing and processing a subset/group of time series elements in the time series sequence at once (i.e., together, in parallel, concurrently). A subject composite element (token) in the composite sequence is generated based on an aggregate transformation of a corresponding subset/group of time series elements in the time series sequence, a process known as tokenization. Each composite element (token) can have multiple channels, as depicted in FIG. 1.

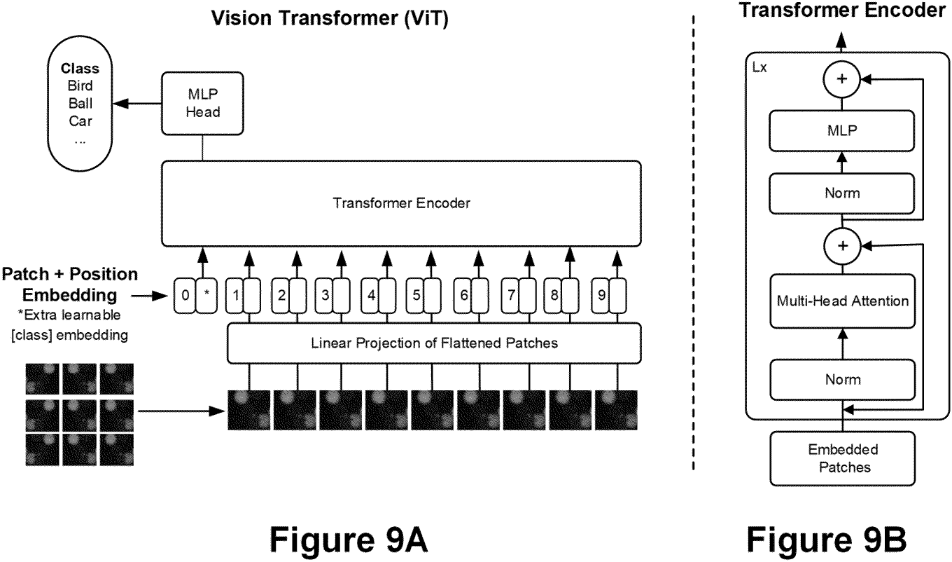

In another implementation, a linear projection layer is trained to learn weights that apply the respective aggregate transformations and generate the composite sequence. Turning to FIG. 9A, an example linear projection layer is shown as part of the Vision Transformer (ViT) model. In some implementations, the time series elements (e.g., sequencing images 102) are divided into patches/tiles for processing by the linear projection layer.

The composite sequence is processed as an aggregate to generate a base call sequence 132 that has respective base calls (e.g., base call 1-base call 151 (BC1-BC151)) for the respective bases in the read for the respective sequencing cycles (e.g., cycle 1-cycle 151 (c1-c151)). As used herein, “aggregate processing of the composite sequence” refers to the Transformer-based base caller 122 processing the entire token sequence 112 at once (i.e., together, in parallel, concurrently) and generating a base call out output for each token in the token sequence 112 at once (i.e., together, in parallel, concurrently).

In one implementation depicted in FIG. 1, the composite sequence is generated for the read based on the respective aggregate transformations of respective sliding windows of the time series elements in the time series sequence. In one implementation, the respective sliding windows have overlapping time series elements. In another implementation, the respective sliding windows are non-overlapping. In another implementation, each of the respective sliding windows has N time series elements, where N is an integer greater than 1 (e.g., N can be 3-, 5-, 9-kmer, and so on.). In one implementation, the linear projection layer is trained to learn the weights that apply the respective aggregate transformations on the respective sliding windows of the time series elements in the time series sequence and to generate the composite sequence.

In another implementation, the respective base calls for the respective bases in the read are concurrently generated.

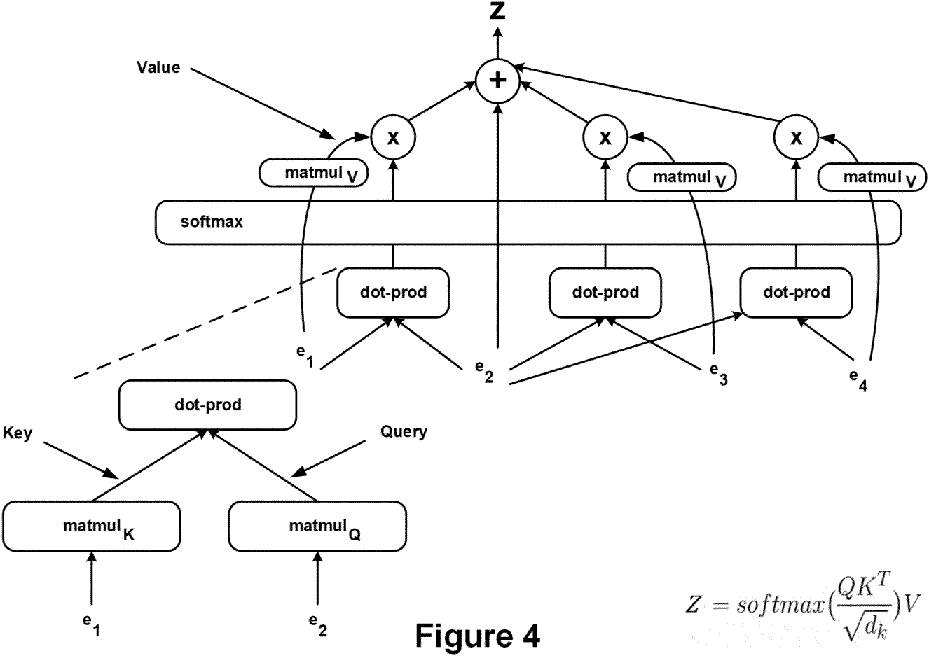

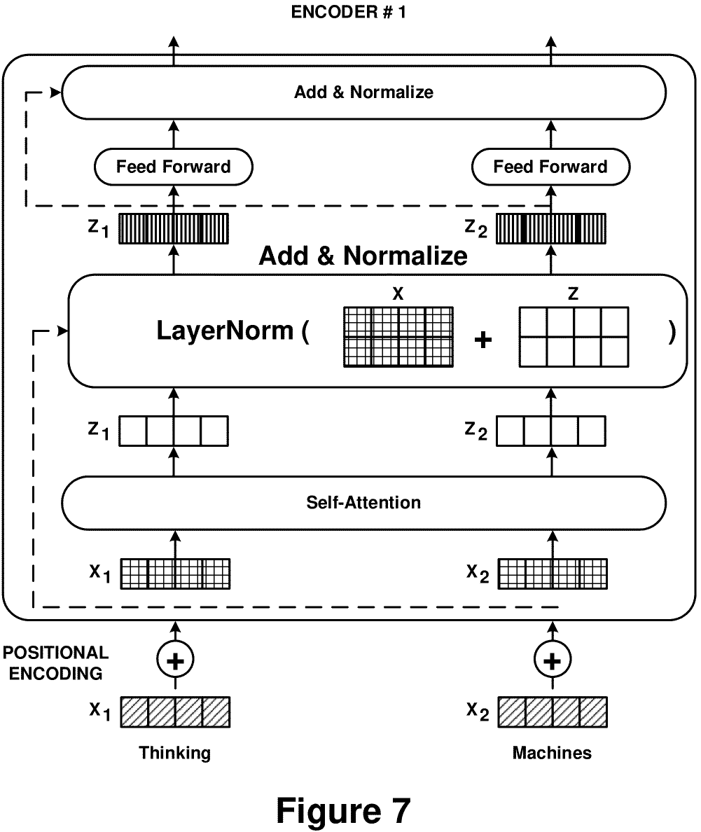

In another implementation, a multi-headed attention encoder (e.g., FIGS. 7, 9A-B) is trained to process the composite sequence as the aggregate and to generate an alternative representation of the composite sequence. In one example, the alternative representation can have many dimensions, such as 147×64 dimensions. In one implementation, the multi-headed attention encoder is trained using self-attention (e.g., FIG. 4). In another implementation, the multi-headed attention encoder is trained using cross-attention.

Implementations of the technology disclosed do not use the decoder and only use the encoder because intensities from sequencing instruments, and base calls that are generated as output by the disclosed base calling model(s) have the same time ordering.

In another implementation, an output layer (e.g., softmax layer) is trained to process the alternative representation of the composite sequence and generate the base call sequence. In one implementation, the output layer is trained to concurrently generate base-wise classification likelihoods for each composite element in the composite sequence. In one implementation, a base call for a subject base in the read is determined based on a maximum base-wise classification likelihood generated by the output layer for a corresponding composite element in the composite sequence. For example, the base call sequence 132 can have a dimensionality of 151×4, where 151 corresponds to the number of sequencing cycles in the read, and 4 corresponds to the four nucleotide base likelihoods for each base call.

In another implementation, the multi-headed attention encoder is trained to correct for systematic errors in cluster amplification that are encoded in the read. In one implementation, the systematic errors include phasing and prephasing errors. In another implementation, the systematic errors include context dependent intensity modulations.

In another implementation, the multi-headed attention encoder is trained to analyze backward and forward flanking composite elements in conjunction with a subject composite element being analyzed in the composite sequence. In one implementation, a forward mask of the multi-headed attention encoder is deactivated to account for the forward flanking composite elements. In yet another implementation, the multi-headed attention encoder is trained on read data from multiple human gene sources. In one implementation, the trained multi-headed attention encoder is tested on read data from multiple bacteria gene sources.

In another implementation, the time series sequence has a dimensionality of L × C, where L is a number of bases in the read, and C is a number of channels. In one implementation, the composite sequence has a dimensionality of (L-(W-1) × (W × C), where W is a size of the respective sliding windows. In one implementation, the base call sequence has a dimensionality of (L-(W-1) × 4. In another implementation, the composite sequence has a dimensionality of L × C in dependence upon zero padded composite elements. In one implementation, the base call sequence has a dimensionality of L × C.

In yet another implementation, the multi-headed attention encoder uses a positional embedding (e.g., depicted in FIG. 9A) to determine relative inter-element spatial arrangement of composite elements in the composite sequence. In one implementation, the positional embedding is learned during training of the multi-headed attention encoder. In another implementation, the positional embedding is provided as a Fourier embedding.

In another implementation, we disclose a computer-implemented method of base calling in which a time series sequence of a read is accessed. The respective time series elements in the time series sequence represent respective bases in the read. A composite sequence for the read is generated based on respective aggregate transformations of respective sliding windows of time series elements in the time series sequence A subject composite element in the composite sequence is generated based on an aggregate transformation of a corresponding window of time series elements in the time series sequence. Then the composite sequence is processed as an aggregate and a base call sequence is generated that has respective base calls for the respective bases in the read.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Transformers originate from the field of natural language processing (NLP), but can be used in computer vision and many other fields. Neural networks process input in series and weight relationships by distance in the series. Transformers can process input in parallel and do not necessarily weight by distance. For example, in natural language processing, neural networks process a sentence from beginning to end with the weights of words close to each other being higher than those further apart. This leaves the end of the sentence very disconnected from the beginning causing an effect called the vanishing gradient problem. Transformers look at each word in parallel and determine weights for the relationships to each of the other words in the sentence. These relationships are called hidden states because they are later condensed for use into one vector called the context vector. Transformers can be used in addition to neural networks. This architecture is described here.

### Encoder-Decoder Architecture

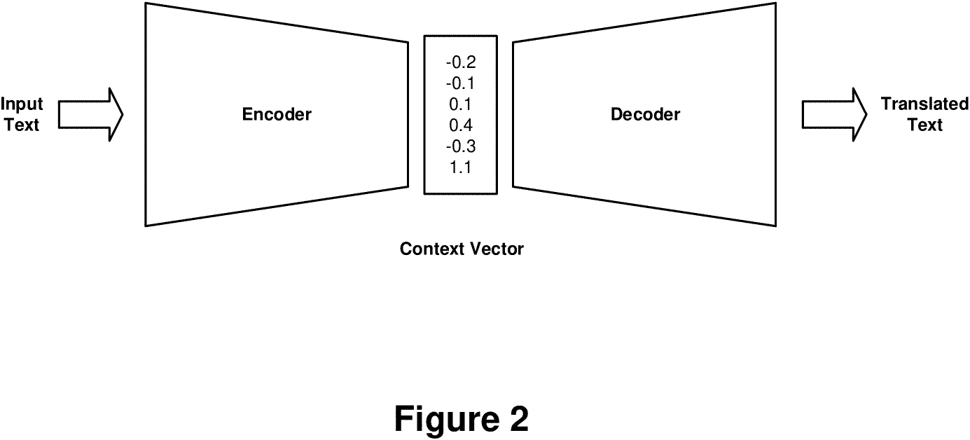

FIG. 2 is a schematic representation of an encoder-decoder architecture. This architecture is often used for NLP and has two main building blocks. The first building block is the encoder that encodes an input into a fixed-size vector. In the system we describe here, the encoder is based on a recurrent neural network (RNN). At each time step, t, a hidden state of time step, t-1, is combined with the input value at time step t to compute the hidden state at timestep t. The hidden state at the last time step, encoded in a context vector, contains relationships encoded at all previous time steps. For NLP, each step corresponds to a word. Then the context vector contains information about the grammar and the sentence structure. The context vector can be considered a low-dimensional representation of the entire input space. For NLP, the input space is a sentence, and a training set consists of many sentences.

The context vector is then passed to the second building block, the decoder. For translation, the decoder has been trained on a second language. Conditioned on the input context vector, the decoder generates an output sequence. At each time step, t, the decoder is fed the hidden state of time step, t-1, and the output generated at time step, t-1. The first hidden state in the decoder is the context vector, generated by the encoder. The context vector is used by the decoder to perform the translation.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using only RNNs in an encoder-decoder architecture.

### Attention Mechanism

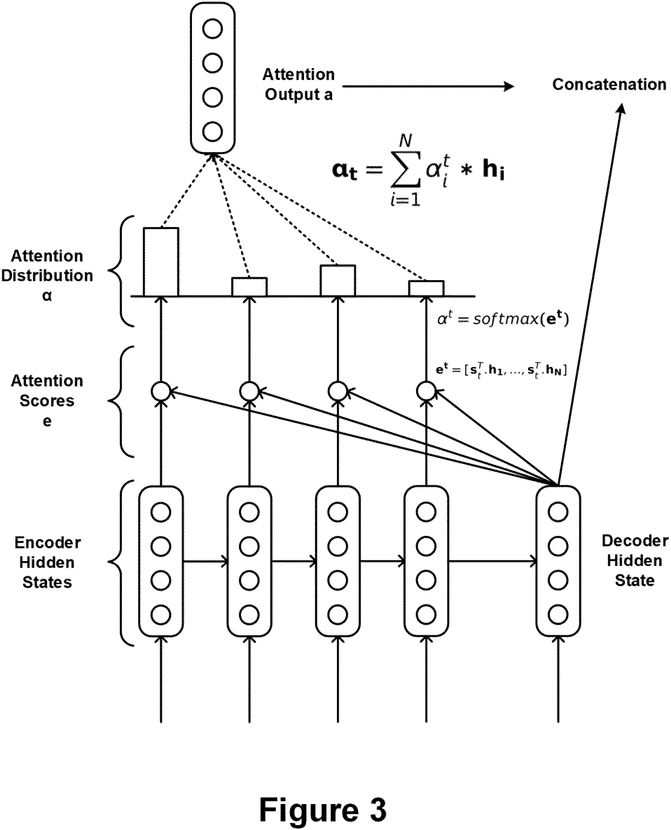

Attention mechanisms distinguish Transformers from other machine learning models. The attention mechanism provides a solution for the vanishing gradient problem. FIG. 3 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is be based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentence.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighting the different values (multiplicative attention).

### Embeddings

For most machine learning models, the input to the model needs to be numerical. The input to a translation model is a sentence, and words are not numerical. multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For NLP, this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by only using a vector of three elements. Also, similar colors, have similar representation vectors.

For NLP, embeddings based on context, as opposed to words, are small and can be trained. The reasoning behind this concept is that words with similar meanings occur in similar contexts. Different methods take the context of words into account. Some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vector using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

Multi-headed attention is executed in the Transformer. FIG. 4 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that are most relevant to the initial query.

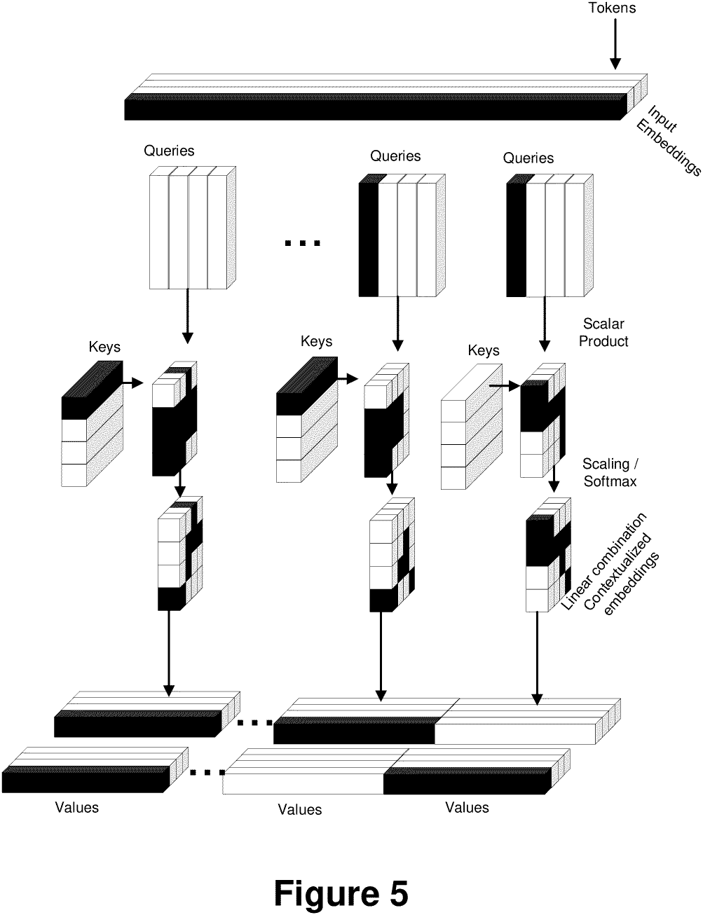

FIG. 5 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as

$$\sqrt{d k} .$$

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$\begin{matrix}\text{Attention} \left(\text{Q,} \text{K,} \text{V}\right) = \text{SoftMax} \left(\frac{Q {K}^{T}}{\sqrt{d k}}\right) V \\ \text{where Q, K, V are computed as:} \\ X ⋅ {W}_{Q} , X ⋅ {W}_{K} , X ⋅ {W}_{V}\end{matrix}$$

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space into the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as MultiHeadAttention (Q, K, V) = [head₁, ..., headₕ]W₀ where headᵢ = Attention

$$\left(Q {W}_{i}^{Q} , K {W}_{i}^{K} , V {W}_{i}^{V}\right)$$

The outputs of all heads are concatenated together and projected again using the learned weights matrix W₀ to match the dimensions expected by the next block of heads or the output of the Transformer. Using the multi-head attention instead of the simpler scaled dot-product attention enables Transformers to jointly attend to information from different representation subspaces at different positions.

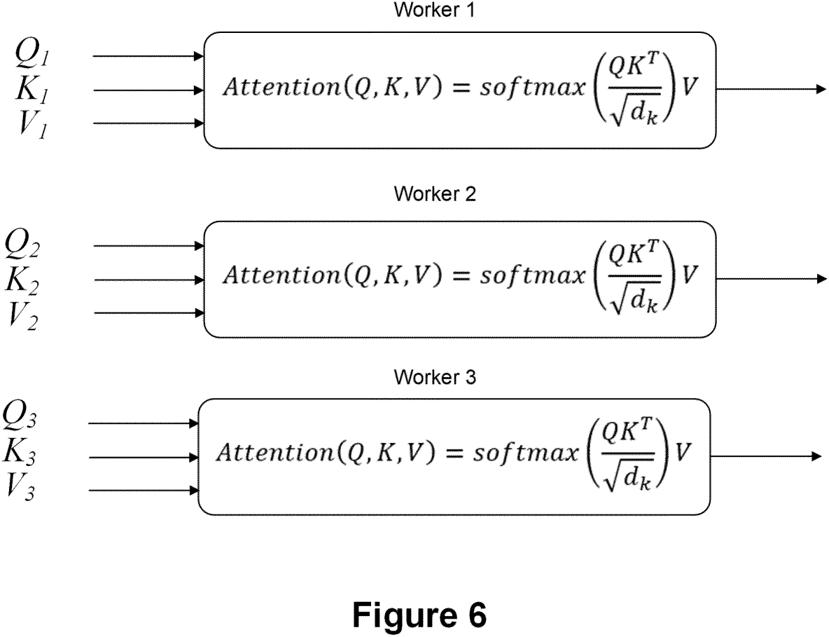

As shown in FIG. 6, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

$$a ⋅ b ⋅ c$$

For matrices of shape (a, b) and (c, d), to obtain values Q, K, V, we need to compute the operations:

$$\text{X} ⋅ {\text{W}}_{\text{Q}} , \text{X} ⋅ {\text{W}}_{\text{K}} , \text{X} ⋅ \text{WV}$$

The matrix X is of shape (n, d) where n is the number of patches and d is the hidden vector dimension. The weights W_{Q}, W_{K}, Wu are all of shape (d, d). Omitting the constant factor 3, the resulting complexity is:

$$\text{n} ⋅ {\text{d}}^{2}$$

We can proceed to the estimation of the complexity of the attention function itself, i.e., of

SoftMax

$$\left(\frac{Q {K}^{T}}{\sqrt{d k}}\right) V .$$

The matrices Q and K are both of shape (n, d). The transposition operation does not influence the asymptotic complexity of computing the dot product of matrices of shapes (n, d) • (d, n), therefore its complexity is:

$${\text{n}}^{2} ⋅ \text{d}$$

Scaling by a constant factor of

$$\sqrt{d k} ,$$

where dk is the dimension of the keys vector, as well as applying the SoftMax function, both have the complexity of a • b for a matrix of shape (a, b), hence they do not influence the asymptotic complexity. Lastly the dot product SoftMax

$$\left(\frac{Q {K}^{T}}{\sqrt{d k}}\right) ⋅ V$$

is between matrices of shapes (n, n) and (n, d) and so its complexity is:

$${\text{n}}^{2} ⋅ \text{d}$$

The final asymptotic complexity of scaled dot-product attention is obtained by summing the complexities of computing Q, K, V, and of the attention function

$$\text{n} ⋅ {\text{d}}^{2} + {\text{n}}^{2} ⋅ \text{d} \text{.}$$

The asymptotic complexity of multi-head attention is the same since the original input matrix X is projected into h matrices of shapes

$$\left(n , \frac{d}{h}\right) ,$$

where h is the number of heads. From the view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

FIG. 7 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is only compared to the keys of previous output sequence elements. The elements further in the sequence are not known yet, as they still must be predicted. No information about these output elements may be used.

### Encoder-Decoder Blocks of Transformer

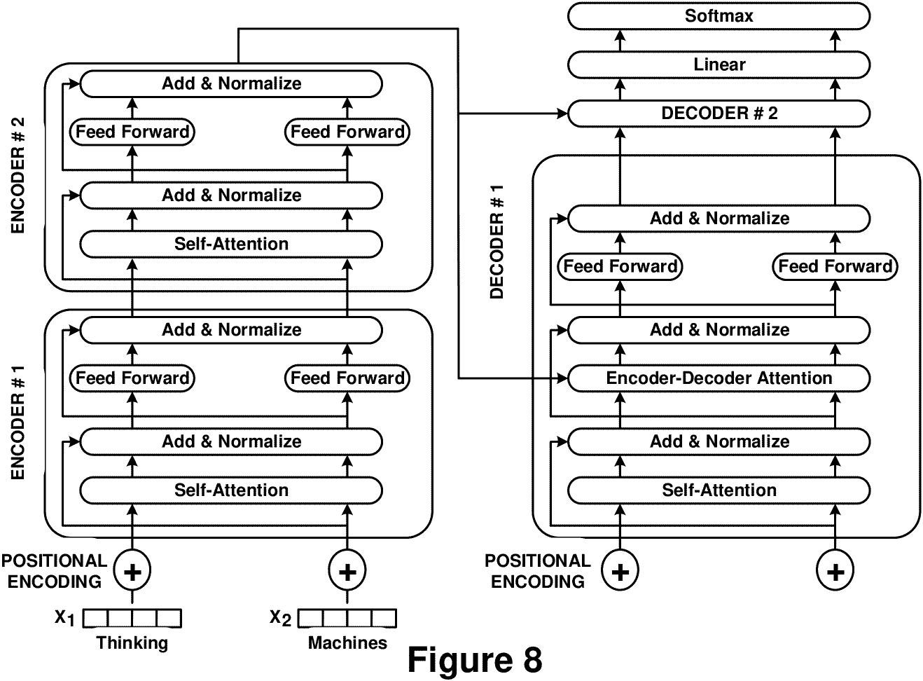

FIG. 8 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, only an encoder is needed. This is true for both document classification and name entity recognition. In these cases, the encoded input vectors are the input of the feed-forward layer and the SoftMax layer. Transformer models have been extensively applied in different NLP fields, such as translation, document summarization, speech recognition, and named entity recognition. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

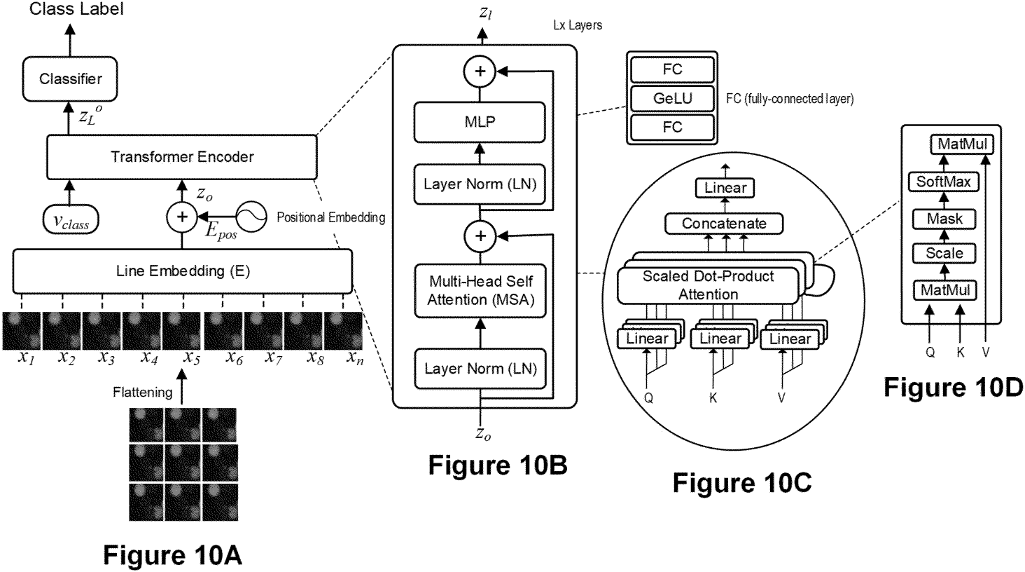

Transformers were originally developed for NLP and worked with sequences of words. In image classification, we often have a single input image in which the pixels are in a sequence. To reduce the computation required, Vision Transformers (ViTs) cut the input image into a set of fixed-sized patches of pixels. The patches are often 16 × 16 pixels. They are treated much like words in NLP Transformers. ViTs are depicted in FIGS. 9A, 9B, 10A, 10B, 10C, and 10D. Unfortunately, important positional information is lost because image sets are position-invariant. This problem is solved by adding a learned positional encoding into the image patches.

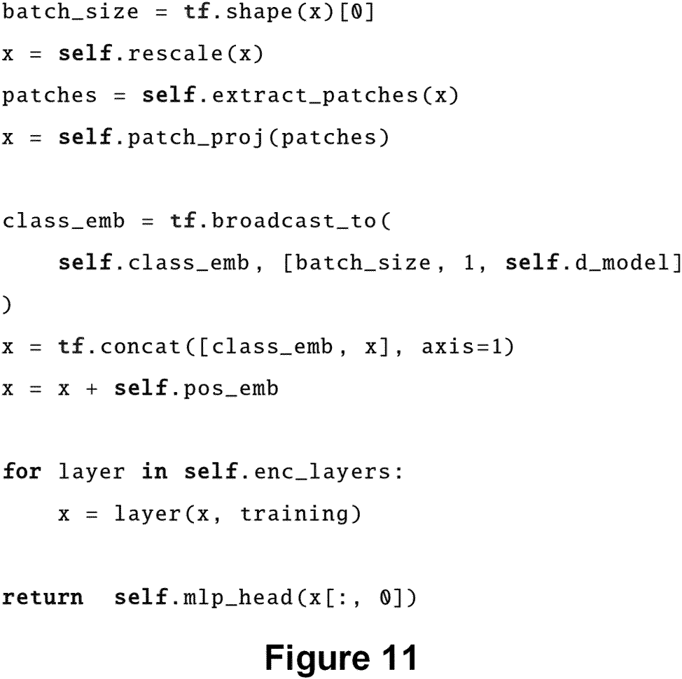

The computations of the ViT architecture can be summarized as follows. The first layer of a ViT extracts a fixed number of patches from an input image (FIG. 9A). The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 11.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block in FIG. 9B, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite directions of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

### Performance Results as Objective Indicia of Inventiveness and Non-Obviousness

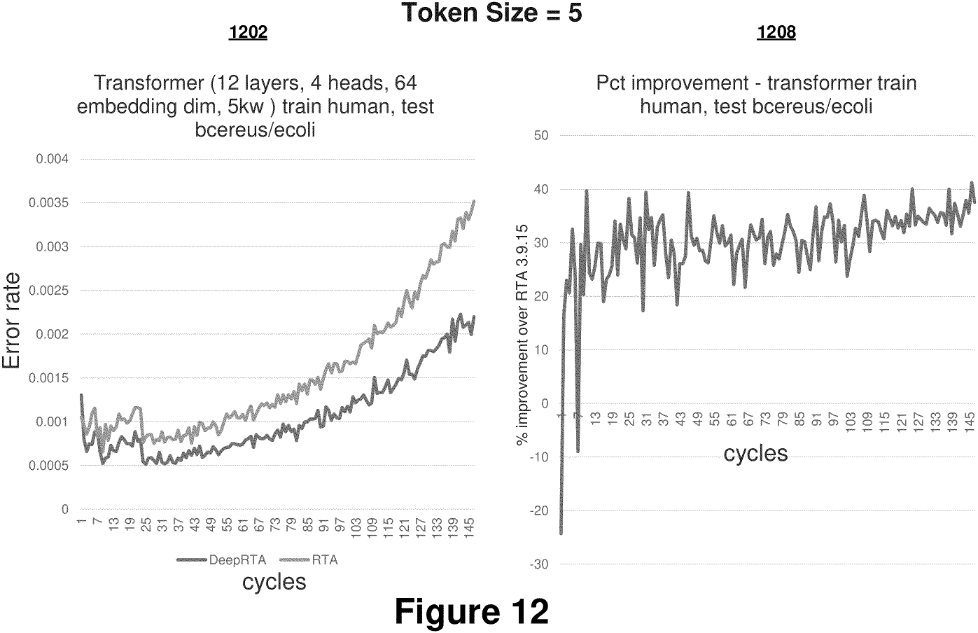

FIG. 12 has a left plot 1202 that depicts base calling error rate measured on a ground truth dataset comparing RTA base caller (a non-neural network-based base caller) and the disclosed Transformer-based base caller 122 with full read context, and a right plot 1208, which is the same as the left plot 1202 but measures the fractional base calling error rate improvement across sequencing cycles by the disclosed Transformer-based base caller 122.

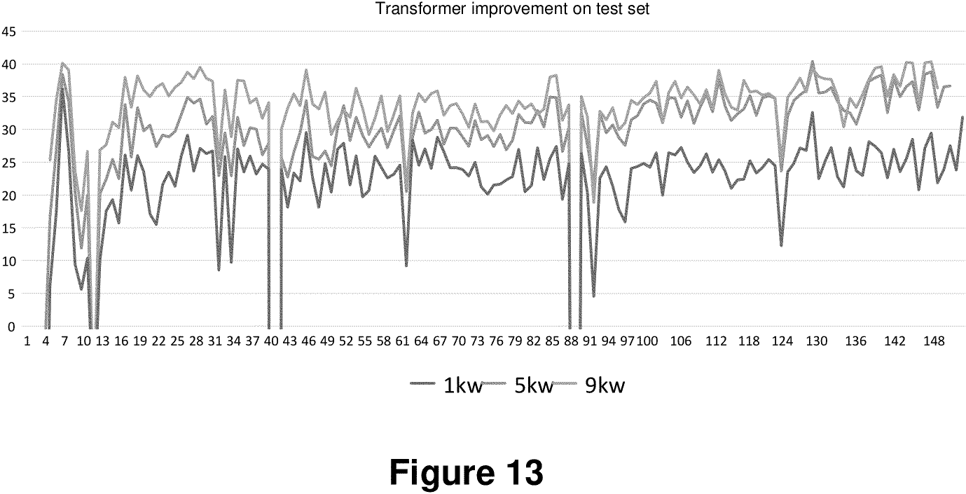

FIG. 13 illustrates a hyperparameter scan of the length of the k-mer used as the input to the disclosed Transformer-based base caller 122. A larger k-mer window shows larger improvements (grey curve is for a 9 cycle k-mer window).

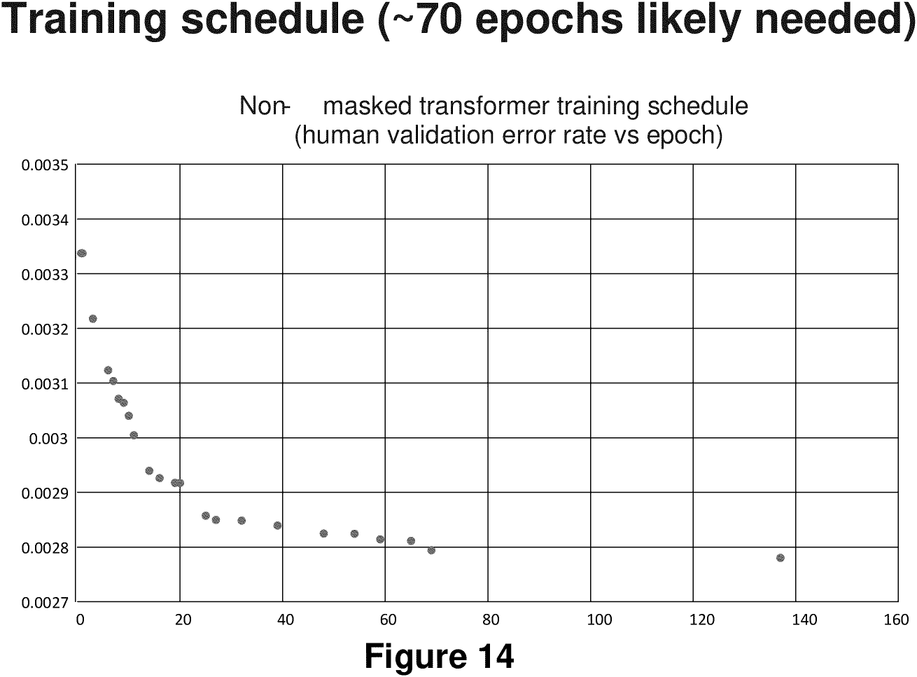

FIG. 14 represents the training loss across epochs through the dataset and that ~70 epochs are needed to train the disclosed Transformer-based base caller 122.

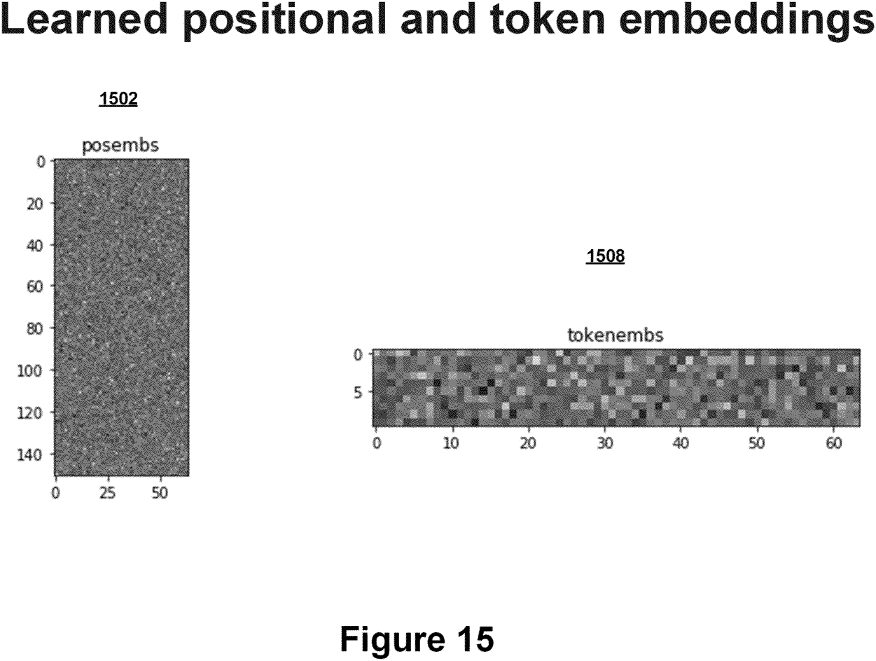

FIG. 15 depicts the learned feature maps that represent the positional 1502 and token embeddings 1508 used as the input of the disclosed Transformer-based base caller 122.

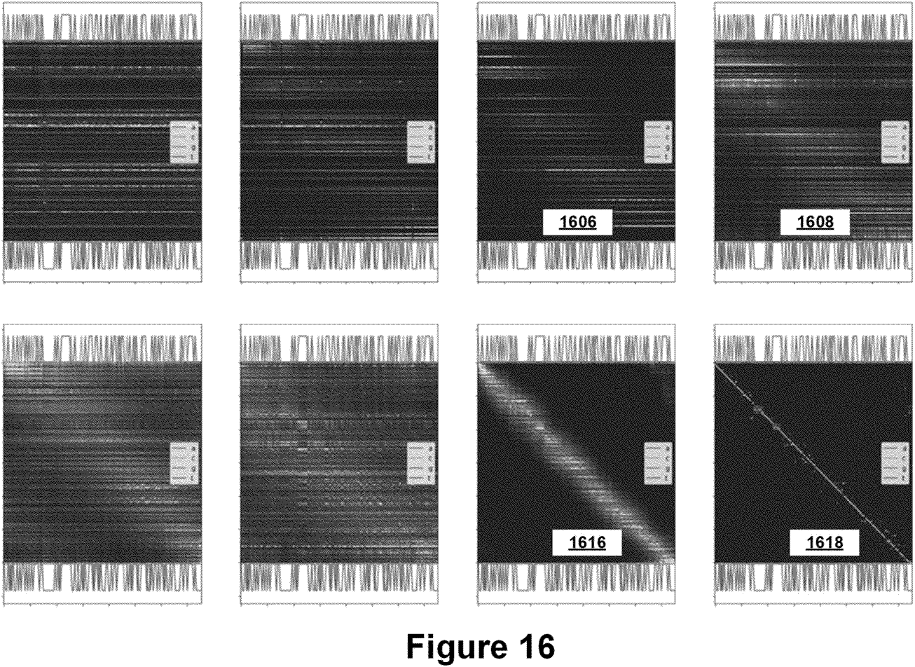

FIG. 16 describes the attention maps for a 2 Layers (rows), 4-Heads per Layer (columns) implementation of the disclosed Transformer-based base caller 122 trained on full sequence context. The maps in this plot originate from a single sequence shown in the top and bottom tracks above and below each plot (ACGT as Blue, Orange, Green, Red). Each attention plot is a 151×151 map of which sequencing cycles the disclosed Transformer-based base caller 122 considers in order to call the bases at the output. We note that for most maps, the disclosed Transformer-based base caller 122 looks at a large amount of context (large horizontal streaks in layer 1 heads 1-4 and layer 2 heads 1-2), possibly collecting long-range statistics about the read.

Turning to plots 1606, 1608, 1616, and 1618 for Layer 2, Heads 3 and 4 in FIG. 16. We note that, for these maps, the disclosed Transformer-based base caller 122 pays close attention to the immediate context of the current base being called (represented on the diagonal line). Layer 2, Head 3 shows that a local context of about 30 bases (15 up and downstream) is useful to the disclosed Transformer-based base caller 122. Layer 2, Head 4 shows that the disclosed Transformer-based base caller 122 looks at specific cycles that are upstream and downstream of the current called base/current cycle to adjust its decisions (e.g., up to 15 bases upstream or downstream).

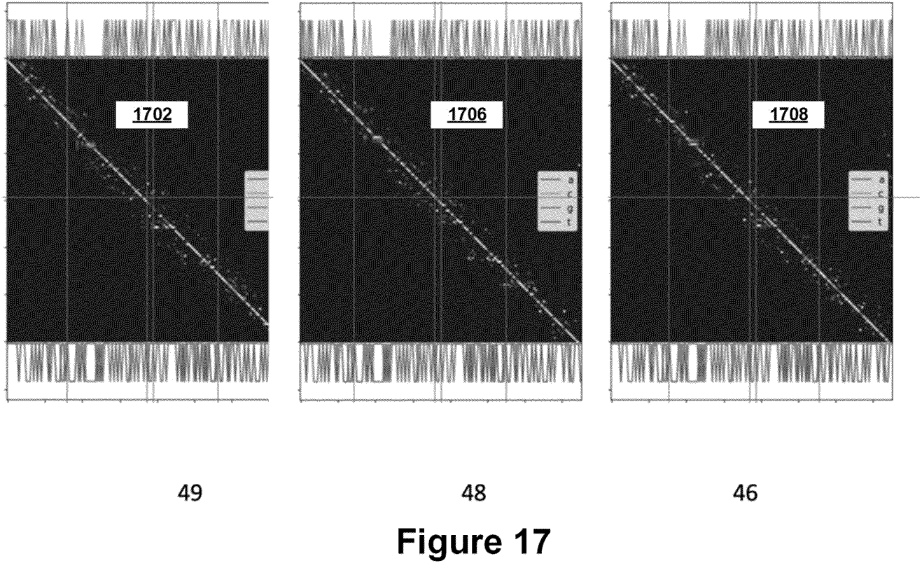

FIG. 17 shows Layer 2, Head 4 attention maps 1702, 1706, and 1708 of the disclosed Transformer-based base caller 122 from 3 different clusters that have the same sequence but are offset by a few sequencing cycles in the sequencing run. We note that the attention maps 1702, 1706, and 1708 are shifted according to the sequence in the read and thus the disclosed Transformer-based base caller 122 captures sequence-specific features of the reads with these attention maps 1702, 1706, and 1708. The vertical blue lines are here as guides to the eye for tracking how specific features move together with the read sequence.

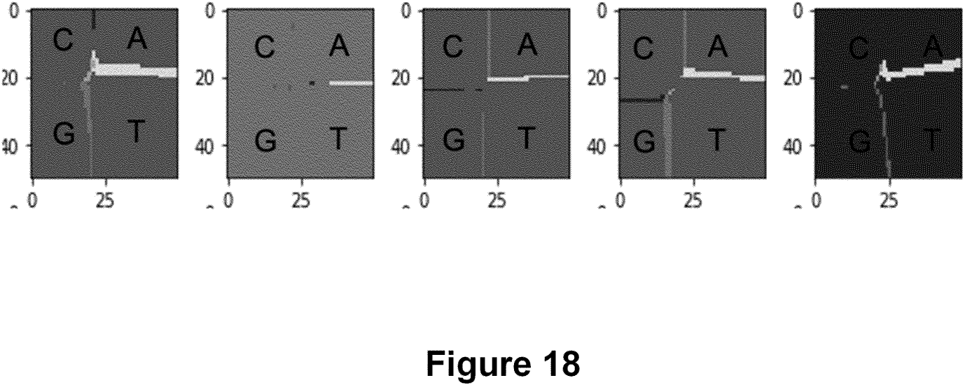

FIG. 18 has plots that show the decision boundary differentials for a few cycles of a sequence if we force the disclosed Transformer-based base caller 122 to only consider the center cycle in Layer 2, Head 4. This shows that the disclosed Transformer-based base caller 122 relies considerably on the feature map generated by the Layer 2, Head 4 to produce its output base calls.

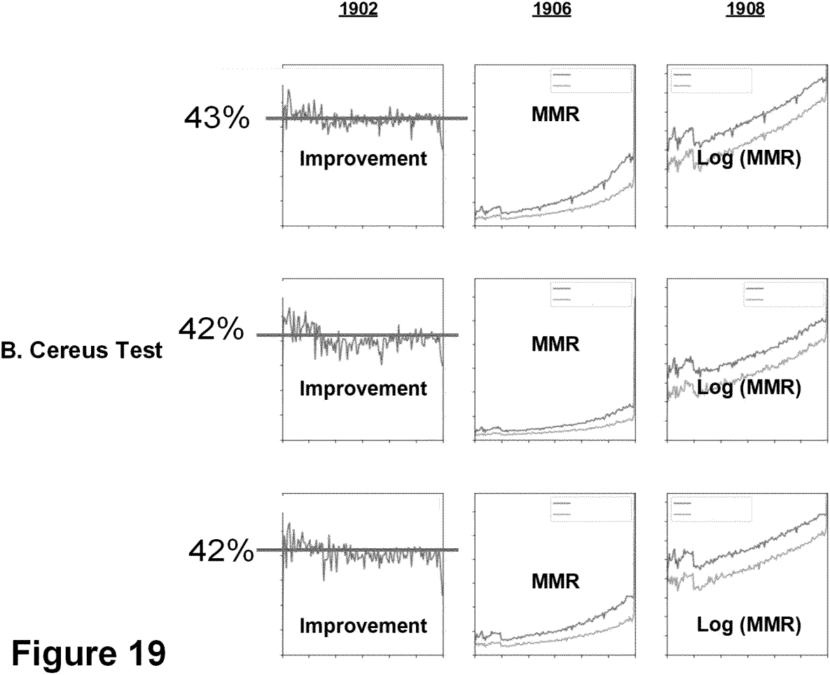

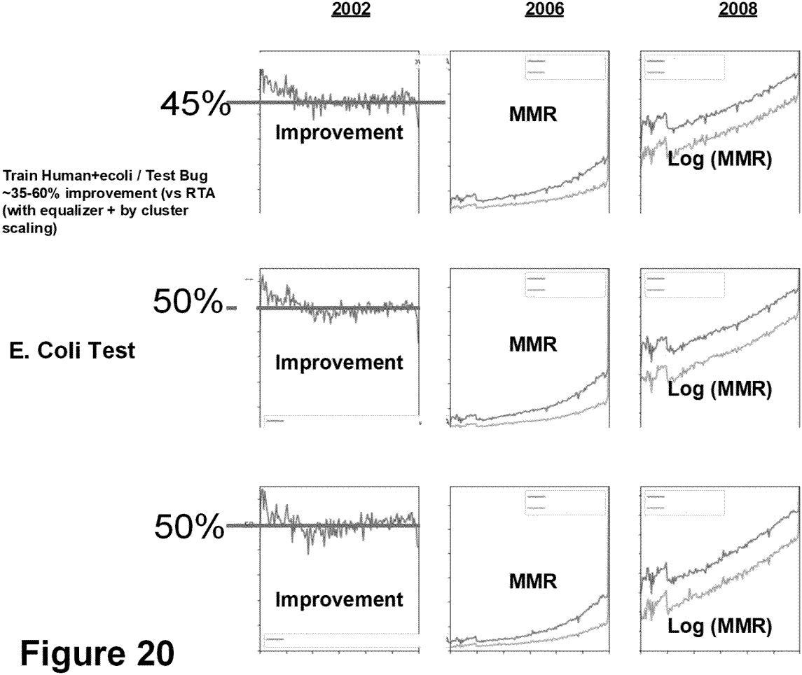

FIGS. 19 and 20 have plots that show the improvement in the base calling error rate of the disclosed Transformer-based base caller 122 v/s the RTA base caller (a non-neural network-based base caller). In FIGS. 19 and 20, the first column plots 1902 and 2002 indicate the percent improvement in the base calling error rate of the disclosed Transformer-based base caller 122 v/s the RTA base caller. The second column plots 1906 and 2006 are the same as the first column plots 1902 and 2002 but plot the two base calling error rates vs sequencing cycles separately. The third column plots 1908 and 2008 show the same plots as the second column but with the y-axis on the log scale.

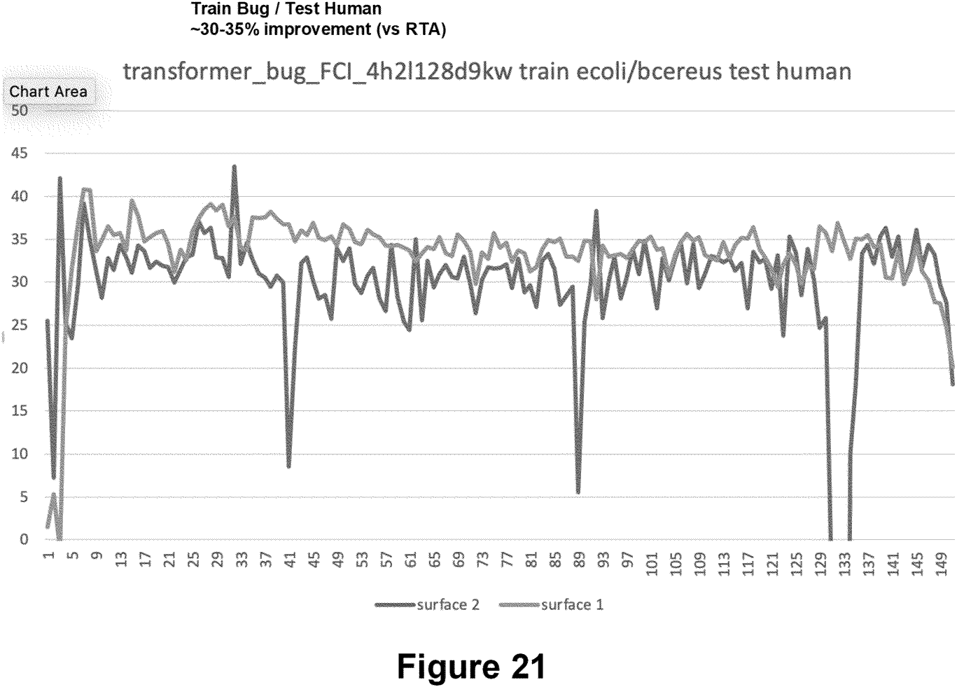

FIG. 21 shows the improvement obtained when training on a bacterial dataset and testing on a human dataset. This plot demonstrates that the disclosed Transformer-based base caller 122 is not overfitting to the genome significantly (in other words, not learning the sequence of the human genome).

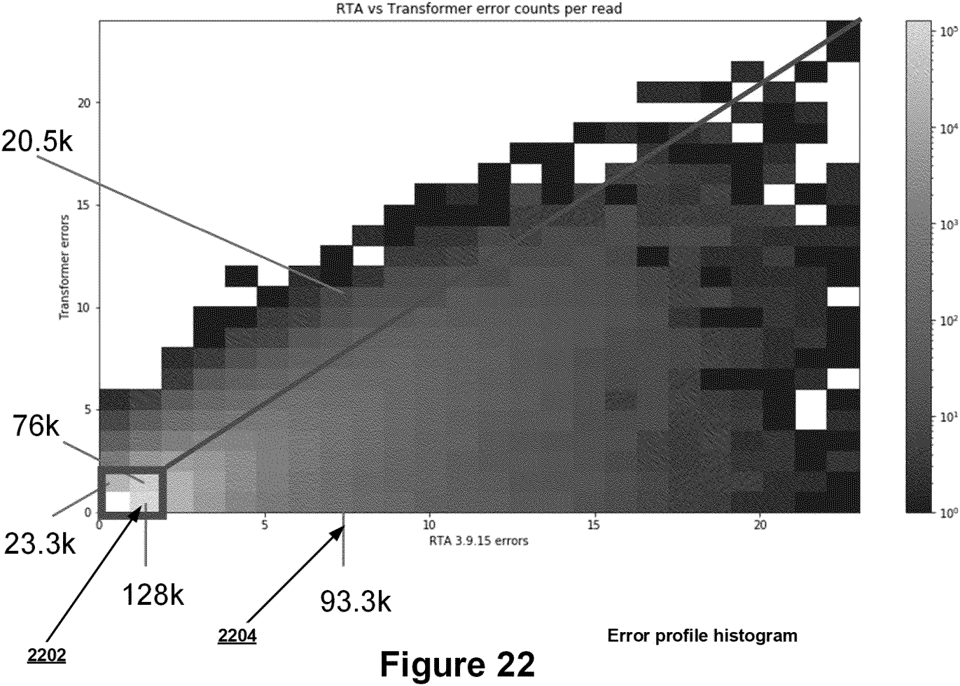

FIG. 22 shows where most of the gains of the disclosed Transformer-based base caller 122 in base calling error rate come from in terms of counts of errors per read. As illustrated in FIG. 22, a large fraction comes from the correction 2202 of single errors (128k), a secondary large fraction comes from the correction 2204 of multiple errors in a read.

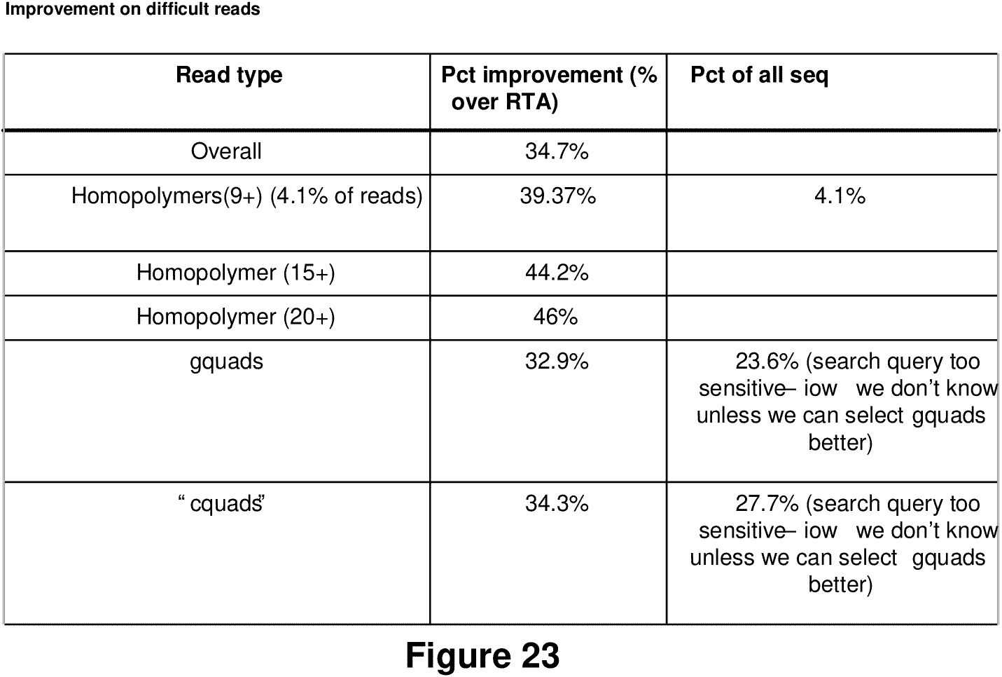

The disclosed Transformer-based base caller 122 accurately base calls homopolymers. FIG. 23 shows the improvements measured in homopolymer over the whole testing dataset. As homopolymers get longer, they have a higher chance of having a polymerase slippage, and hence accumulate sudden large amounts of phasing. The table in FIG. 23 shows that as the homopolymers get longer, the improvement in the base calling error rate of the disclosed Transformer-based base caller 122 also becomes larger. That is, the base call accuracy of the disclosed Transformer-based base caller 122 increases as the length of the homopolymers increases. Homopolymers are the cause of many systematic errors in base calling and therefore a significant fraction of the genome is affected by base calling errors resulting from homopolymers. Since more sequencing does not help with recovering those genomic regions that experience base calling errors caused homopolymers, it becomes all the more important to accurately base call homopolymers.

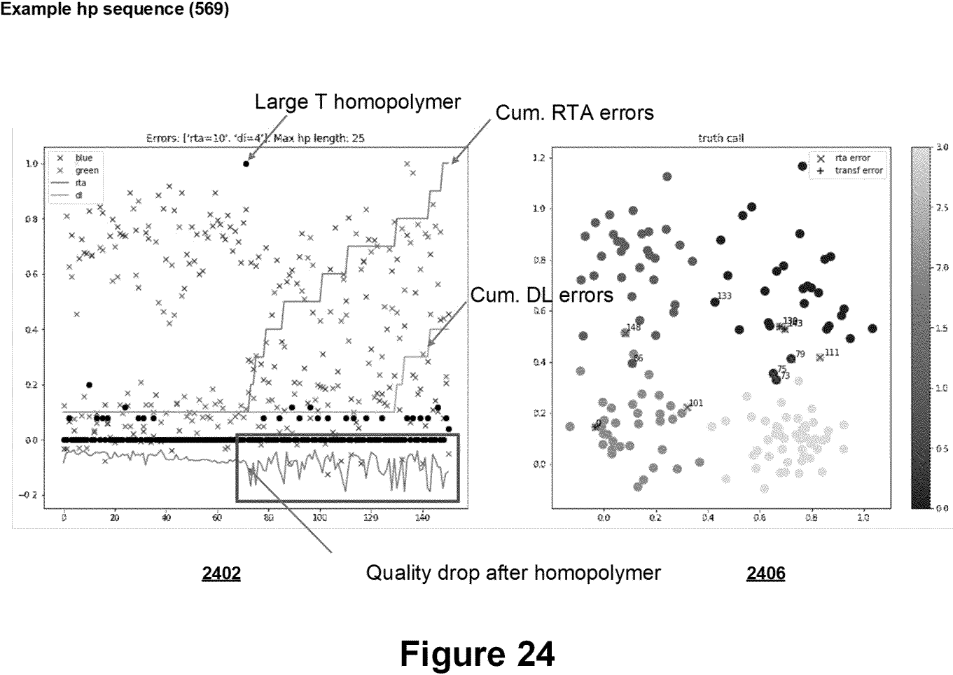

FIG. 24 figure shows one sequence with a large homopolymer that has a sequence-specific error profile. At cycles 47-72 in the run, this sequence has a large 25 bp T homopolymer. On the left plot 2402, the black dots represent the length of homopolymers contained in the sequence under consideration. We note the large 25 bp homopolymer at cycle 72. The blue line shows the cumulative count of errors made by the RTA. The orange curve shows the same but are errors made by the disclosed Transformer-based base caller 122. There are significantly fewer errors made by the disclosed Transformer-based base caller 122 as compared to the RTA.

The disclosed Transformer-based base caller 122 is able to correct for significant changes in phasing on this and other similar sequences, which is different from other deep learning-based base callers. In the green track, we note the associated drop in quality, immediately after the homopolymer. Blue and red crosses indicate the intensities of images fed as input to the disclosed Transformer-based base caller 122.

In the upper right part of FIG. 24, we see an alignment of 3 sequences: from top to bottom the sequence called by the RTA, the truth sequence, and the sequence called by the disclosed Transformer-based base caller 122. Vertical “|” characters indicate where the base pairs in the sequence match. “x” shows where there is a mismatch. For each mismatch in this alignment, there is a corresponding “up step” in the cumulative error curve on the preceding plot.

In the right plot 2406, we see where the errors were incurred in the context of the input intensity space.

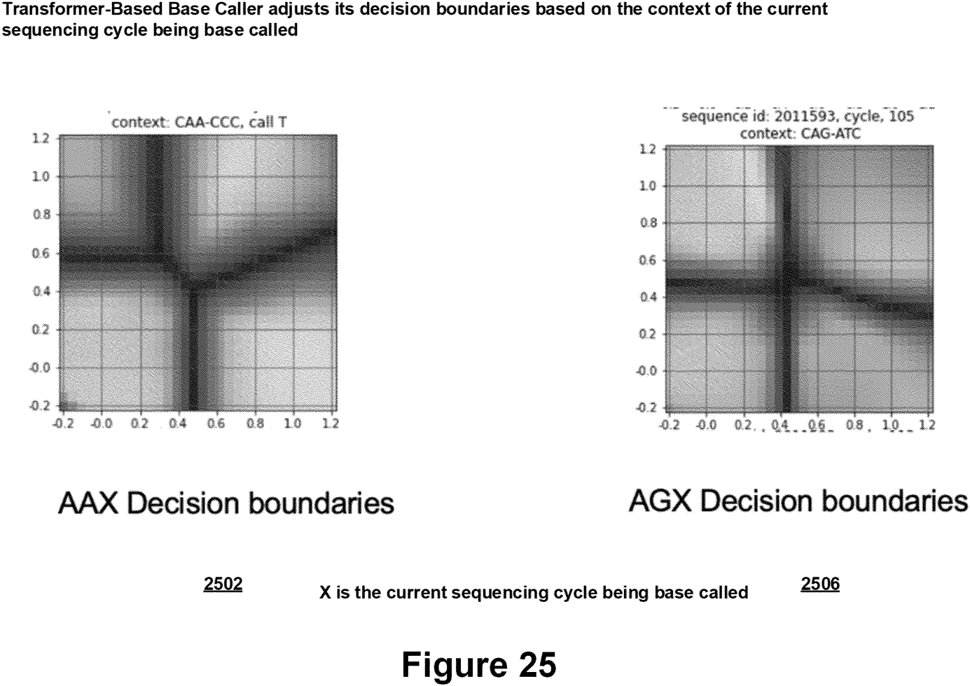

FIG. 25 has plots 2502 and 2506 that show how the disclosed Transformer-based base caller 122 adjusts its decision boundaries strongly based on the preceding 2 bases of context. Sequencing System

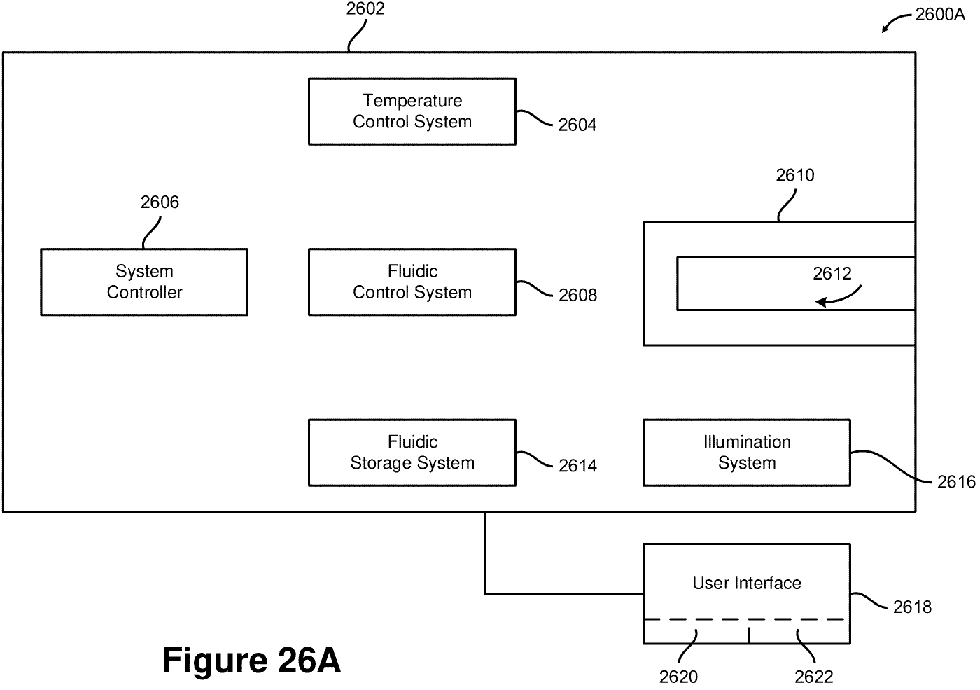

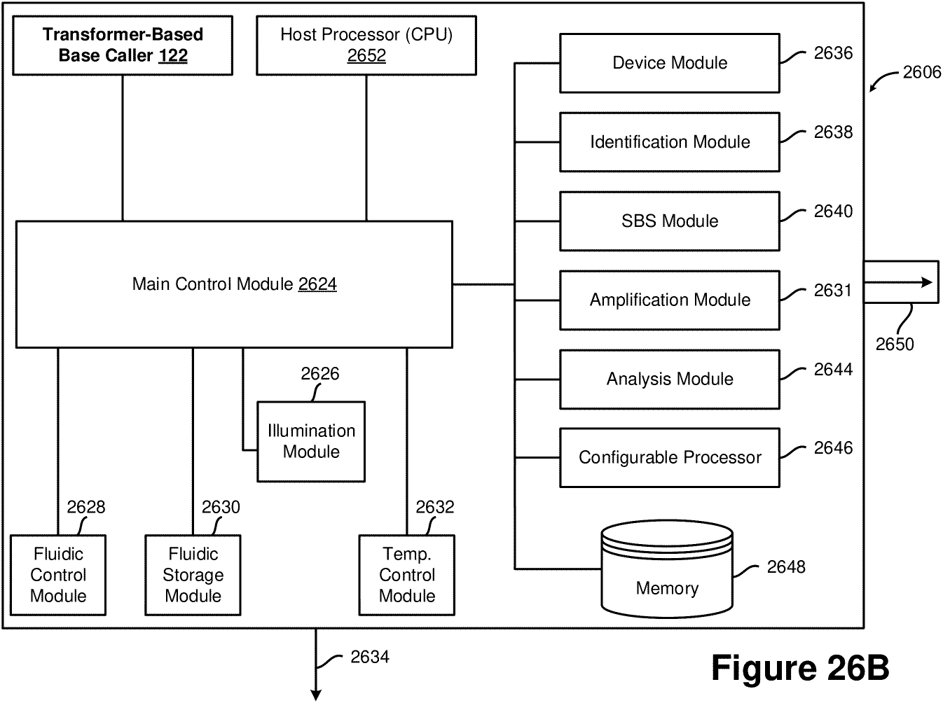

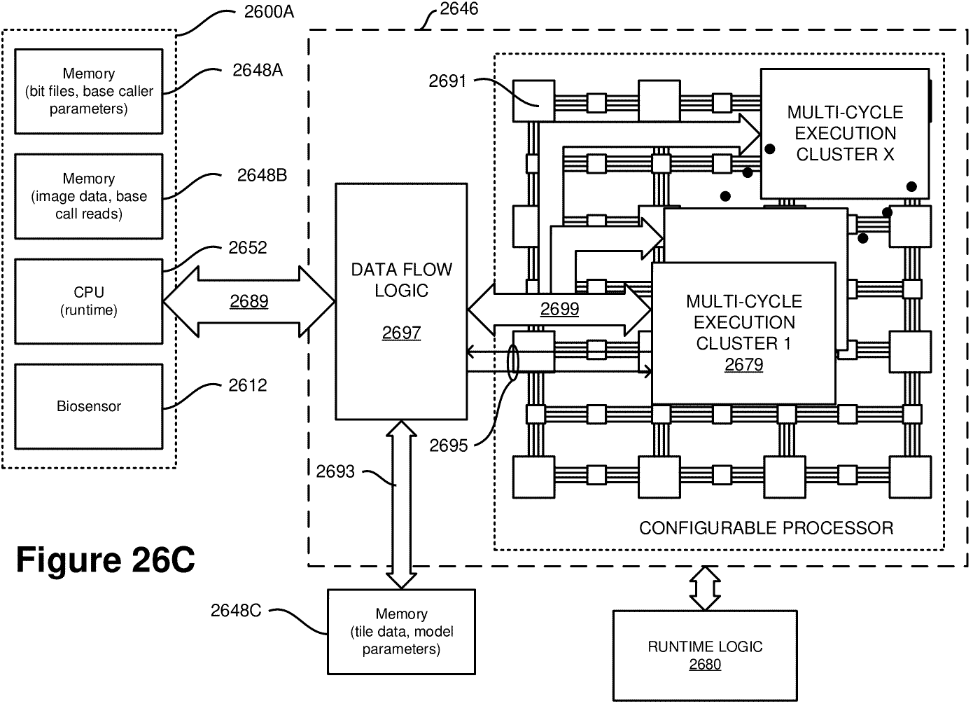

FIGS. 26A and 26B depict one implementation of a sequencing system 2600A. The sequencing system 2600A comprises a configurable processor 2646. The configurable processor 2646 implements the base calling techniques disclosed herein. The sequencing system is also referred to as a “sequencer.”

The sequencing system 2600A can operate to obtain any information or data that relates to at least one of a biological or chemical substance. In some implementations, the sequencing system 2600A is a workstation that may be similar to a bench-top device or desktop computer. For example, a majority (or all) of the systems and components for conducting the desired reactions can be within a common housing 2602.

In particular implementations, the sequencing system 2600A is a nucleic acid sequencing system configured for various applications, including but not limited to de novo sequencing, resequencing of whole genomes or target genomic regions, and metagenomics. The sequencer may also be used for DNA or RNA analysis. In some implementations, the sequencing system 2600A may also be configured to generate reaction sites in a biosensor. For example, the sequencing system 2600A may be configured to receive a sample and generate surface attached clusters of clonally amplified nucleic acids derived from the sample. Each cluster may constitute or be part of a reaction site in the biosensor.

The exemplary sequencing system 2600A may include a system receptacle 2610 (or system interface) that is configured to interact with a biosensor 2612 to perform desired reactions within the biosensor 2612. In the following description with respect to FIG. 26A, the biosensor 2612 is loaded into the system receptacle 2610. However, it is understood that a cartridge that includes the biosensor 2612 may be inserted into the system receptacle 2610 and in some states the cartridge can be removed temporarily or permanently. As described above, the cartridge may include, among other things, fluidic control and fluidic storage components.

In particular implementations, the sequencing system 2600A is configured to perform a large number of parallel reactions within the biosensor 2612. The biosensor 2612 includes one or more reaction sites where desired reactions can occur. The reaction sites may be, for example, immobilized to a solid surface of the biosensor or immobilized to beads (or other movable substrates) that are located within corresponding reaction chambers of the biosensor. The reaction sites can include, for example, clusters of clonally amplified nucleic acids. The biosensor 2612 may include a solid-state imaging device (e.g., CCD or CMOS imager) and a flow cell mounted thereto. The flow cell may include one or more flow channels that receive a solution from the sequencing system 2600A and direct the solution toward the reaction sites. Optionally, the biosensor 2612 can be configured to engage a thermal element for transferring thermal energy into or out of the flow channel.

The sequencing system 2600A may include various components, assemblies, and systems (or sub-systems) that interact with each other to perform a predetermined method or assay protocol for biological or chemical analysis. For example, the sequencing system 2600A includes a system controller 2606 that may communicate with the various components, assemblies, and sub-systems of the sequencing system 2600A and also the biosensor 2612. For example, in addition to the system receptacle 2610, the sequencing system 2600A may also include a fluidic control system 2608 to control the flow of fluid throughout a fluid network of the sequencing system 2600A and the biosensor 2612; a fluid storage system 2614 that is configured to hold all fluids (e.g., gas or liquids) that may be used by the bioassay system; a temperature control system 2604 that may regulate the temperature of the fluid in the fluid network, the fluid storage system 2614, and/or the biosensor 2612; and an illumination system 2616 that is configured to illuminate the biosensor 2612. As described above, if a cartridge having the biosensor 2612 is loaded into the system receptacle 2610, the cartridge may also include fluidic control and fluidic storage components.

Also shown, the sequencing system 2600A may include a user interface 2618 that interacts with the user. For example, the user interface 2618 may include a display 2620 to display or request information from a user and a user input device 2622 to receive user inputs. In some implementations, the display 2620 and the user input device 2622 are the same device. For example, the user interface 2618 may include a touch-sensitive display configured to detect the presence of an individual’s touch and also identify a location of the touch on the display. However, other user input devices may be used, such as a mouse, touchpad, keyboard, keypad, handheld scanner, voice-recognition system, motion-recognition system, and the like. As will be discussed in greater detail below, the sequencing system 2600A may communicate with various components, including the biosensor 2612 (e.g., in the form of a cartridge), to perform the desired reactions. The sequencing system 2600A may also be configured to analyze data obtained from the biosensor to provide a user with desired information.

The system controller 2606 may include any processor-based or microprocessor-based system, including systems using microcontrollers, reduced instruction set computers (RISC), application specific integrated circuits (ASICs), field programmable gate array (FPGAs), coarse-grained reconfigurable architectures (CGRAs), logic circuits, and any other circuit or processor capable of executing functions described herein. The above examples are exemplary only, and are thus not intended to limit in any way the definition and/or meaning of the term system controller. In the exemplary implementation, the system controller 2606 executes a set of instructions that are stored in one or more storage elements, memories, or modules in order to at least one of obtain and analyze detection data. Detection data can include a plurality of sequences of pixel signals, such that a sequence of pixel signals from each of the millions of sensors (or pixels) can be detected over many base calling cycles. Storage elements may be in the form of information sources or physical memory elements within the sequencing system 2600A.

The set of instructions may include various commands that instruct the sequencing system 2600A or biosensor 2612 to perform specific operations such as the methods and processes of the various implementations described herein. The set of instructions may be in the form of a software program, which may form part of a tangible, non-transitory computer readable medium or media. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

The software may be in various forms such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the sequencing system 2600A, processed in response to user inputs, or processed in response to a request made by another processing machine (e.g., a remote request through a communication link). In the illustrated implementation, the system controller 2606 includes an analysis module 2644. In other implementations, system controller 2606 does not include the analysis module 2644 and instead has access to the analysis module 2644 (e.g., the analysis module 2644 may be separately hosted on cloud).

The system controller 2606 may be connected to the biosensor 2612 and the other components of the sequencing system 2600A via communication links. The system controller 2606 may also be communicatively connected to off-site systems or servers. The communication links may be hardwired, corded, or wireless. The system controller 2606 may receive user inputs or commands, from the user interface 2618 and the user input device 2622.

The fluidic control system 2608 includes a fluid network and is configured to direct and regulate the flow of one or more fluids through the fluid network. The fluid network may be in fluid communication with the biosensor 2612 and the fluid storage system 2614. For example, select fluids may be drawn from the fluid storage system 2614 and directed to the biosensor 2612 in a controlled manner, or the fluids may be drawn from the biosensor 2612 and directed toward, for example, a waste reservoir in the fluid storage system 2614. Although not shown, the fluidic control system 2608 may include flow sensors that detect a flow rate or pressure of the fluids within the fluid network. The sensors may communicate with the system controller 2606.

The temperature control system 2604 is configured to regulate the temperature of fluids at different regions of the fluid network, the fluid storage system 2614, and/or the biosensor 2612. For example, the temperature control system 2604 may include a thermocycler that interfaces with the biosensor 2612 and controls the temperature of the fluid that flows along the reaction sites in the biosensor 2612. The temperature control system 2604 may also regulate the temperature of solid elements or components of the sequencing system 2600A or the biosensor 2612. Although not shown, the temperature control system 2604 may include sensors to detect the temperature of the fluid or other components. The sensors may communicate with the system controller 2606.

The fluid storage system 2614 is in fluid communication with the biosensor 2612 and may store various reaction components or reactants that are used to conduct the desired reactions therein. The fluid storage system 2614 may also store fluids for washing or cleaning the fluid network and biosensor 2612 and for diluting the reactants. For example, the fluid storage system 2614 may include various reservoirs to store samples, reagents, enzymes, other biomolecules, buffer solutions, aqueous, and non-polar solutions, and the like. Furthermore, the fluid storage system 2614 may also include waste reservoirs for receiving waste products from the biosensor 2612. In implementations that include a cartridge, the cartridge may include one or more of a fluid storage system, fluidic control system or temperature control system. Accordingly, one or more of the components set forth herein as relating to those systems can be contained within a cartridge housing. For example, a cartridge can have various reservoirs to store samples, reagents, enzymes, other biomolecules, buffer solutions, aqueous, and non-polar solutions, waste, and the like. As such, one or more of a fluid storage system, fluidic control system or temperature control system can be removably engaged with a bioassay system via a cartridge or other biosensor.

The illumination system 2616 may include a light source (e.g., one or more LEDs) and a plurality of optical components to illuminate the biosensor. Examples of light sources may include lasers, arc lamps, LEDs, or laser diodes. The optical components may be, for example, reflectors, dichroics, beam splitters, collimators, lenses, filters, wedges, prisms, mirrors, detectors, and the like. In implementations that use an illumination system, the illumination system 2616 may be configured to direct an excitation light to reaction sites. As one example, fluorophores may be excited by green wavelengths of light, as such the wavelength of the excitation light may be approximately 2627 nm. In one implementation, the illumination system 2616 is configured to produce illumination that is parallel to a surface normal of a surface of the biosensor 2612. In another implementation, the illumination system 2616 is configured to produce illumination that is off-angle relative to the surface normal of the surface of the biosensor 2612. In yet another implementation, the illumination system 2616 is configured to produce illumination that has plural angles, including some parallel illumination and some off-angle illumination.

The system receptacle 2610 is configured to engage the biosensor 2612 in at least one of a mechanical, electrical, and fluidic manner. The system receptacle 2610 may hold the biosensor 2612 in a desired orientation to facilitate the flow of fluid through the biosensor 2612. The system receptacle 2610 may also include electrical contacts that are configured to engage the biosensor 2612 so that the sequencing system 2600A may communicate with the biosensor 2612 and/or provide power to the biosensor 2612. Furthermore, the system receptacle 2610 may include fluidic ports (e.g., nozzles) that are configured to engage the biosensor 2612. In some implementations, the biosensor 2612 is removably coupled to the system receptacle 2610 in a mechanical manner, in an electrical manner, and also in a fluidic manner.

In addition, the sequencing system 2600A may communicate remotely with other systems or networks or with other bioassay systems. Detection data obtained by the bioassay system(s) 2600A may be stored in a remote database.

FIG. 26B is a block diagram of a system controller 2606 that can be used in the system of FIG. 26A. In one implementation, the system controller 2606 includes one or more processors or modules that can communicate with one another. Each of the processors or modules may include an algorithm (e.g., instructions stored on a tangible and/or non-transitory computer readable storage medium) or sub-algorithms to perform particular processes. The system controller 2606 is illustrated conceptually as a collection of modules, but may be implemented utilizing any combination of dedicated hardware boards, DSPs, processors, etc. Alternatively, the system controller 2606 may be implemented utilizing an off-the-shelf PC with a single processor or multiple processors, with the functional operations distributed between the processors. As a further option, the modules described below may be implemented utilizing a hybrid configuration in which certain modular functions are performed utilizing dedicated hardware, while the remaining modular functions are performed utilizing an off-the-shelf PC and the like. The modules also may be implemented as software modules within a processing unit.

During operation, a communication port 2650 may transmit information (e.g., commands) to or receive information (e.g., data) from the biosensor 2612 (FIG. 26A) and/or the sub-systems 2608, 2614, 2604 (FIG. 26A). In implementations, the communication port 2650 may output a plurality of sequences of pixel signals. A communication link 2634 may receive user input from the user interface 2618 (FIG. 26A) and transmit data or information to the user interface 2618. Data from the biosensor 2612 or sub-systems 2608, 2614, 2604 may be processed by the system controller 2606 in real-time during a bioassay session. Additionally or alternatively, data may be stored temporarily in a system memory during a bioassay session and processed in slower than real-time or off-line operation.

As shown in FIG. 26B, the system controller 2606 may include a plurality of modules 2626-2648 that communicate with a main control module 2624, along with a central processing unit (CPU) 2652. The main control module 2624 may communicate with the user interface 2618 (FIG. 26A). Although the modules 2626-2648 are shown as communicating directly with the main control module 2624, the modules 2626-2648 may also communicate directly with each other, the user interface 2618, and the biosensor 2612. Also, the modules 2626-2648 may communicate with the main control module 2624 through the other modules.

The plurality of modules 2626-2648 include system modules 2628, 2630, 2632, and 2626 that communicate with the sub-systems 2608, 2614, 2604, and 2616, respectively. The fluidic control module 2628 may communicate with the fluidic control system 2608 to control the valves and flow sensors of the fluid network for controlling the flow of one or more fluids through the fluid network. The fluid storage module 2630 may notify the user when fluids are low or when the waste reservoir is at or near capacity. The fluid storage module 2630 may also communicate with the temperature control module 2632 so that the fluids may be stored at a desired temperature. The illumination module 2626 may communicate with the illumination system 2616 to illuminate the reaction sites at designated times during a protocol, such as after the desired reactions (e.g., binding events) have occurred. In some implementations, the illumination module 2626 may communicate with the illumination system 2616 to illuminate the reaction sites at designated angles.

The plurality of modules 2626-2648 may also include a device module 2636 that communicates with the biosensor 2612 and an identification module 2638 that determines identification information relating to the biosensor 2612. The device module 2636 may, for example, communicate with the system receptacle 2610 to confirm that the biosensor has established an electrical and fluidic connection with the sequencing system 2600A. The identification module 2638 may receive signals that identify the biosensor 2612. The identification module 2638 may use the identity of the biosensor 2612 to provide other information to the user. For example, the identification module 2638 may determine and then display a lot number, a date of manufacture, or a protocol that is recommended to be run with the biosensor 2612.

The plurality of modules 2626-2648 also includes an analysis module 2644 (also called signal processing module or signal processor) that receives and analyzes the signal data (e.g., image data) from the biosensor 2612. Analysis module 2644 includes memory (e.g., RAM or Flash) to store detection/image data. Detection data can include a plurality of sequences of pixel signals, such that a sequence of pixel signals from each of the millions of sensors (or pixels) can be detected over many base calling cycles. The signal data may be stored for subsequent analysis or may be transmitted to the user interface 2618 to display desired information to the user. In some implementations, the signal data may be processed by the solid-state imager (e.g., CMOS image sensor) before the analysis module 2644 receives the signal data.

The analysis module 2644 is configured to obtain image data from the light detectors at each of a plurality of sequencing cycles. The image data is derived from the emission signals detected by the light detectors and process the image data for each of the plurality of sequencing cycles through the neural network-based base caller and produce a base call for at least some of the clusters at each of the plurality of sequencing cycle. The light detectors can be part of one or more over-head cameras (e.g., Illumina’s GAIIx’s CCD camera taking images of the clusters on the biosensor 2612 from the top), or can be part of the biosensor 2612 itself (e.g., Illumina’s iSeq’s CMOS image sensors underlying the clusters on the biosensor 2612 and taking images of the clusters from the bottom).

The output of the light detectors is the sequencing images, each depicting intensity emissions of the clusters and their surrounding background. The sequencing images depict intensity emissions generated as a result of nucleotide incorporation in the sequences during the sequencing. The intensity emissions are from associated clusters and their surrounding background. The sequencing images are stored in memory 2648.

Protocol modules 2640 and 2631 communicate with the main control module 2624 to control the operation of the sub-systems 2608, 2614, and 2604 when conducting predetermined assay protocols. The protocol modules 2640 and 2631 may include sets of instructions for instructing the sequencing system 2600A to perform specific operations pursuant to predetermined protocols. As shown, the protocol module may be a sequencing-by-synthesis (SBS) module 2640 that is configured to issue various commands for performing sequencing-by-synthesis processes. In SBS, extension of a nucleic acid primer along a nucleic acid template is monitored to determine the sequence of nucleotides in the template. The underlying chemical process can be polymerization (e.g., as catalyzed by a polymerase enzyme) or ligation (e.g., catalyzed by a ligase enzyme). In a particular polymerase-based SBS implementation, fluorescently labeled nucleotides are added to a primer (thereby extending the primer) in a template dependent fashion such that detection of the order and type of nucleotides added to the primer can be used to determine the sequence of the template. For example, to initiate a first SBS cycle, commands can be given to deliver one or more labeled nucleotides, DNA polymerase, etc., into/through a flow cell that houses an array of nucleic acid templates. The nucleic acid templates may be located at corresponding reaction sites. Those reaction sites where primer extension causes a labeled nucleotide to be incorporated can be detected through an imaging event. During an imaging event, the illumination system 2616 may provide an excitation light to the reaction sites. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for implementations that use reversible termination a command can be given to deliver a deblocking reagent to the flow cell (before or after detection occurs). One or more commands can be given to effect wash(es) between the various delivery steps. The cycle can then be repeated n times to extend the primer by n nucleotides, thereby detecting a sequence of length n. Exemplary sequencing techniques are described, for example, in Bentley et al., Nature 456:53-59 (2008); WO 04/018497; US 7,057,026; WO 91/06678; WO 07/123744; US 7,279,492; US 7,211,414; US 7,265,019; US 7,405,281, and US 2008/014708082, each of which is incorporated herein by reference.

For the nucleotide delivery step of an SBS cycle, either a single type of nucleotide can be delivered at a time, or multiple different nucleotide types (e.g., A, C, T and G together) can be delivered. For a nucleotide delivery configuration where only a single type of nucleotide is present at a time, the different nucleotides need not have distinct labels since they can be distinguished based on temporal separation inherent in the individualized delivery. Accordingly, a sequencing method or apparatus can use single color detection. For example, an excitation source need only provide excitation at a single wavelength or in a single range of wavelengths. For a nucleotide delivery configuration where delivery results in multiple different nucleotides being present in the flow cell at one time, sites that incorporate different nucleotide types can be distinguished based on different fluorescent labels that are attached to respective nucleotide types in the mixture. For example, four different nucleotides can be used, each having one of four different fluorophores. In one implementation, the four different fluorophores can be distinguished using excitation in four different regions of the spectrum. For example, four different excitation radiation sources can be used. Alternatively, fewer than four different excitation sources can be used, but optical filtration of the excitation radiation from a single source can be used to produce different ranges of excitation radiation at the flow cell.

In some implementations, fewer than four different colors can be detected in a mixture having four different nucleotides. For example, pairs of nucleotides can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. Exemplary apparatus and methods for distinguishing four different nucleotides using detection of fewer than four colors are described for example in U.S. Pat. App. Ser. Nos. 61/538,294 and 61/619,878, which are incorporated herein by reference in their entireties. U.S. Application No. 13/624,200, which was filed on Sep. 21, 2012, is also incorporated by reference in its entirety.

The plurality of protocol modules may also include an amplification module 2631 that is configured to issue commands to the fluidic control system 2608 and the temperature control system 2604 for amplifying a product within the biosensor 2612. For example, the biosensor 2612 may be engaged to the sequencing system 2600A. The amplification module 2631 may issue instructions to the fluidic control system 2608 to deliver necessary amplification components to reaction chambers within the biosensor 2612. In other implementations, the reaction sites may already contain some components for amplification, such as the template DNA and/or primers. After delivering the amplification components to the reaction chambers, the amplification module 2631 may instruct the temperature control system 2604 to cycle through different temperature stages according to known amplification protocols. In some implementations, the amplification and/or nucleotide incorporation is performed isothermally.

The SBS module 2640 may issue commands to perform bridge PCR where clusters of clonal amplicons are formed on localized areas within a channel of a flow cell. After generating the amplicons through bridge PCR, the amplicons may be “linearized” to make single stranded template DNA, or sstDNA, and a sequencing primer may be hybridized to a universal sequence that flanks a region of interest. For example, a reversible terminator-based sequencing by synthesis method can be used as set forth above or as follows.

Each base calling or sequencing cycle can extend an sstDNA by a single base which can be accomplished for example by using a modified DNA polymerase and a mixture of four types of nucleotides. The different types of nucleotides can have unique fluorescent labels, and each nucleotide can further have a reversible terminator that allows only a single-base incorporation to occur in each cycle. After a single base is added to the sstDNA, excitation light may be incident upon the reaction sites and fluorescent emissions may be detected. After detection, the fluorescent label and the terminator may be chemically cleaved from the sstDNA. Another similar base calling or sequencing cycle may follow. In such a sequencing protocol, the SBS module 2640 may instruct the fluidic control system 2608 to direct a flow of reagent and enzyme solutions through the biosensor 2612. Exemplary reversible terminator-based SBS methods which can be utilized with the apparatus and methods set forth herein are described in U.S. Pat. Application Publication No. 2007/0166705A1, U.S. Pat. Application Publication No. 2006/0188901A1, U.S. Pat. No. 7,057,026 U.S. Pat. Application Publication No. 2006/0240439A1, U.S. Pat. Application Publication No. 2006/02814714709 A1, PCT Publication No. WO 05/065814, U.S. Pat. Application Publication No. 2005/014700900 A1, PCT Publication No. WO 06/08B199 and PCT Publication No. WO 07/01470251, each of which is incorporated herein by reference in its entirety. Exemplary reagents for reversible terminator-based SBS are described in US 7,541,444; US 7,057,026; US 7,414,14716; US 7,427,673; US 7,566,537; US 7,592,435 and WO 07/14835368, each of which is incorporated herein by reference in its entirety.

In some implementations, the amplification and SBS modules may operate in a single assay protocol where, for example, template nucleic acid is amplified and subsequently sequenced within the same cartridge.

The sequencing system 2600A may also allow the user to reconfigure an assay protocol. For example, the sequencing system 2600A may offer options to the user through the user interface 2618 for modifying the determined protocol. For example, if it is determined that the biosensor 2612 is to be used for amplification, the sequencing system 2600A may request a temperature for the annealing cycle. Furthermore, the sequencing system 2600A may issue warnings to a user if a user has provided user inputs that are generally not acceptable for the selected assay protocol.

In implementations, the biosensor 2612 includes millions of sensors (or pixels), each of which generates a plurality of sequences of pixel signals over successive base calling cycles. The analysis module 2644 detects the plurality of sequences of pixel signals and attributes them to corresponding sensors (or pixels) in accordance to the row-wise and/or column-wise location of the sensors on an array of sensors.

FIG. 26C is a simplified block diagram of a system for analysis of sensor data from the sequencing system 2600A, such as base call sensor outputs. In the example of FIG. 26C, the system includes the configurable processor 2646. The configurable processor 2646 can execute a base caller (e.g., the neural network-based base caller ) in coordination with a runtime program/logic 2680 executed by the central processing unit (CPU) 2652 (i.e., a host processor). The sequencing system 2600A comprises the biosensor 2612 and flow cells. The flow cells can comprise one or more tiles in which clusters of genetic material are exposed to a sequence of cluster flows used to cause reactions in the clusters to identify the bases in the genetic material. The sensors sense the reactions for each cycle of the sequence in each tile of the flow cell to provide tile data. Genetic sequencing is a data intensive operation, which translates base call sensor data into sequences of base calls for each cluster of genetic material sensed in during a base call operation.

The system in this example includes the CPU 2652, which executes a runtime program/logic 2680 to coordinate the base call operations, memory 2648B to store sequences of arrays of tile data, base call reads produced by the base calling operation, and other information used in the base call operations. Also, in this illustration the system includes memory 2648A to store a configuration file (or files), such as FPGA bit files, and model parameters for the neural networks used to configure and reconfigure the configurable processor 2646, and execute the neural networks. The sequencing system 2600A can include a program for configuring a configurable processor and in some implementations a reconfigurable processor to execute the neural networks.

The sequencing system 2600A is coupled by a bus 2689 to the configurable processor 2646. The bus 2689 can be implemented using a high throughput technology, such as in one example bus technology compatible with the PCIe standards (Peripheral Component Interconnect Express) currently maintained and developed by the PCI-SIG (PCI Special Interest Group). Also in this example, a memory 2648A is coupled to the configurable processor 2646 by bus 2693. The memory 2648A can be on-board memory, disposed on a circuit board with the configurable processor 2646. The memory 2648A is used for high speed access by the configurable processor 2646 of working data used in the base call operation. The bus 2693 can also be implemented using a high throughput technology, such as bus technology compatible with the PCIe standards.

Configurable processors, including field programmable gate arrays FPGAs, coarse grained reconfigurable arrays CGRAs, and other configurable and reconfigurable devices, can be configured to implement a variety of functions more efficiently or faster than might be achieved using a general purpose processor executing a computer program. Configuration of configurable processors involves compiling a functional description to produce a configuration file, referred to sometimes as a bitstream or bit file, and distributing the configuration file to the configurable elements on the processor. The configuration file defines the logic functions to be executed by the configurable processor, by configuring the circuit to set data flow patterns, use of distributed memory and other on-chip memory resources, lookup table contents, operations of configurable logic blocks and configurable execution units like multiply-and-accumulate units, configurable interconnects and other elements of the configurable array. A configurable processor is reconfigurable if the configuration file may be changed in the field, by changing the loaded configuration file. For example, the configuration file may be stored in volatile SRAM elements, in non-volatile read-write memory elements, and in combinations of the same, distributed among the array of configurable elements on the configurable or reconfigurable processor. A variety of commercially available configurable processors are suitable for use in a base calling operation as described herein. Examples include Google’s Tensor Processing Unit (TPU)™, rackmount solutions like GX4 Rackmount Series™, GX9 Rackmount Series™, NVIDIA DGX-1™, Microsoft’ Stratix V FPGA™, Graphcore’s Intelligent Processor Unit (IPU)™, Qualcomm’s Zeroth Platform™ with Snapdragon processors™, NVIDIA’s Volta™, NVIDIA’s DRIVE PX™, NVIDIA’s JETSON TX1/TX2 MODULE™, Intel’s Nirvana™, Movidius VPU™, Fujitsu DPI™, ARM’s DynamicIQ™, IBM TrueNorth™, Lambda GPU Server with Testa V100s™, Xilinx Alveo™ U200, Xilinx Alveo™ U2190, Xilinx Alveo™ U280, Intel/Altera Stratix™ GX2800, Intel/Altera Stratix™ GX2800, and Intel Stratix™ GX10M. In some examples, a host CPU can be implemented on the same integrated circuit as the configurable processor.

Implementations described herein implement the neural network-based base caller using the configurable processor 2646. The configuration file for the configurable processor 2646 can be implemented by specifying the logic functions to be executed using a high level description language HDL or a register transfer level RTL language specification. The specification can be compiled using the resources designed for the selected configurable processor to generate the configuration file. The same or similar specification can be compiled for the purposes of generating a design for an application-specific integrated circuit which may not be a configurable processor.

Alternatives for the configurable processor configurable processor 2646, in all implementations described herein, therefore include a configured processor comprising an application specific ASIC or special purpose integrated circuit or set of integrated circuits, or a system-on-a-chip SOC device, or a graphics processing unit (GPU) processor or a coarse-grained reconfigurable architecture (CGRA) processor, configured to execute a neural network based base call operation as described herein.

In general, configurable processors and configured processors described herein, as configured to execute runs of a neural network, are referred to herein as neural network processors.

The configurable processor 2646 is configured in this example by a configuration file loaded using a program executed by the CPU 2652, or by other sources, which configures the array of configurable elements 2691 (e.g., configuration logic blocks (CLB) such as look up tables (LUTs), flip-flops, compute processing units (PMUs), and compute memory units (CMUs), configurable I/O blocks, programmable interconnects), on the configurable processor to execute the base call function. In this example, the configuration includes data flow logic 2697 which is coupled to the buses 2689 and 2693 and executes functions for distributing data and control parameters among the elements used in the base call operation.

Also, the configurable processor 2646 is configured with data flow logic 2697 to execute the neural network-based base caller. The data flow logic 2697 comprises multi-cycle execution clusters (e.g., 2679) which, in this example, includes execution cluster 1 through execution cluster X. The number of multi-cycle execution clusters can be selected according to a trade-off involving the desired throughput of the operation, and the available resources on the configurable processor 2646.

The multi-cycle execution clusters are coupled to the data flow logic 2697 by data flow paths 2699 implemented using configurable interconnect and memory resources on the configurable processor 2646. Also, the multi-cycle execution clusters are coupled to the data flow logic 2697 by control paths 2695 implemented using configurable interconnect and memory resources for example on the configurable processor 2646, which provide control signals indicating available execution clusters, readiness to provide input units for execution of a run of the neural network-based base caller to the available execution clusters, readiness to provide trained parameters for the neural network-based base caller, readiness to provide output patches of base call classification data, and other control data used for execution of the neural network-based base caller.

The configurable processor 2646 is configured to execute runs of the neural network-based base caller using trained parameters to produce classification data for the sensing cycles of the base calling operation. A run of the neural network-based base caller is executed to produce classification data for a subject sensing cycle of the base calling operation. A run of the neural network-based base caller operates on a sequence including a number N of arrays of tile data from respective sensing cycles of N sensing cycles, where the N sensing cycles provide sensor data for different base call operations for one base position per operation in time sequence in the examples described herein. Optionally, some of the N sensing cycles can be out of sequence if needed according to a particular neural network model being executed. The number N can be any number greater than one. In some examples described herein, sensing cycles of the N sensing cycles represent a set of sensing cycles for at least one sensing cycle preceding the subject sensing cycle and at least one sensing cycle following the subject cycle in time sequence. Examples are described herein in which the number N is an integer equal to or greater than five.

The data flow logic 2697 is configured to move tile data and at least some trained parameters of the model parameters from the memory 2648A to the configurable processor 2646 for runs of the neural network-based base caller, using input units for a given run including tile data for spatially aligned patches of the N arrays. The input units can be moved by direct memory access operations in one DMA operation, or in smaller units moved during available time slots in coordination with the execution of the neural network deployed.

Tile data for a sensing cycle as described herein can comprise an array of sensor data having one or more features. For example, the sensor data can comprise two images which are analyzed to identify one of four bases at a base position in a genetic sequence of DNA, RNA, or other genetic material. The tile data can also include metadata about the images and the sensors. For example, in implementations of the base calling operation, the tile data can comprise information about alignment of the images with the clusters such as distance from center information indicating the distance of each pixel in the array of sensor data from the center of a cluster of genetic material on the tile.

During execution of the neural network-based base caller as described below, tile data can also include data produced during execution of the neural network-based base caller, referred to as intermediate data, which can be reused rather than recomputed during a run of the neural network-based base caller. For example, during execution of the neural network-based base caller, the data flow logic 2697 can write intermediate data to the memory 2648A in place of the sensor data for a given patch of an array of tile data. Implementations like this are described in more detail below.

As illustrated, a system is described for analysis of base call sensor output, comprising memory (e.g., 2648A) accessible by the runtime program/logic 2680 storing tile data including sensor data for a tile from sensing cycles of a base calling operation. Also, the system includes a neural network processor, such as configurable processor 2646 having access to the memory. The neural network processor is configured to execute runs of a neural network using trained parameters to produce classification data for sensing cycles. As described herein, a run of the neural network is operating on a sequence of N arrays of tile data from respective sensing cycles of N sensing cycles, including a subject cycle, to produce the classification data for the subject cycle. The data flow logic 2697 is provided to move tile data and the trained parameters from the memory to the neural network processor for runs of the neural network using input units including data for spatially aligned patches of the N arrays from respective sensing cycles of N sensing cycles.

Also, a system is described in which the neural network processor has access to the memory, and includes a plurality of execution clusters, the execution clusters in the plurality of execution clusters configured to execute a neural network. The data flow logic 2697 has access to the memory and to execution clusters in the plurality of execution clusters, to provide input units of tile data to available execution clusters in the plurality of execution clusters, the input units including a number N of spatially aligned patches of arrays of tile data from respective sensing cycles, including a subject sensing cycle, and to cause the execution clusters to apply the N spatially aligned patches to the neural network to produce output patches of classification data for the spatially aligned patch of the subject sensing cycle, where N is greater than 1.

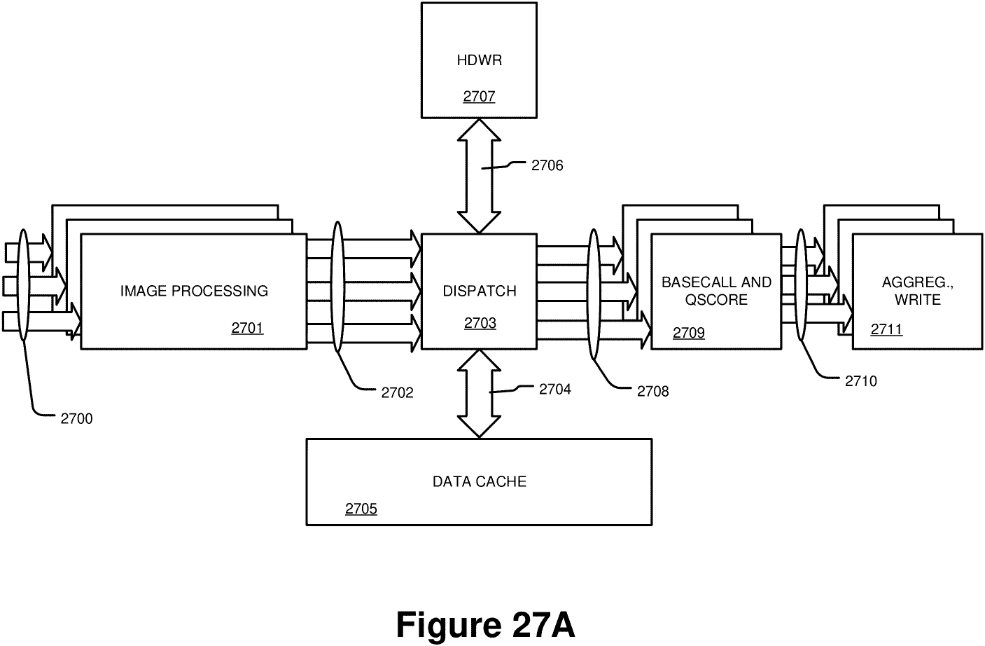

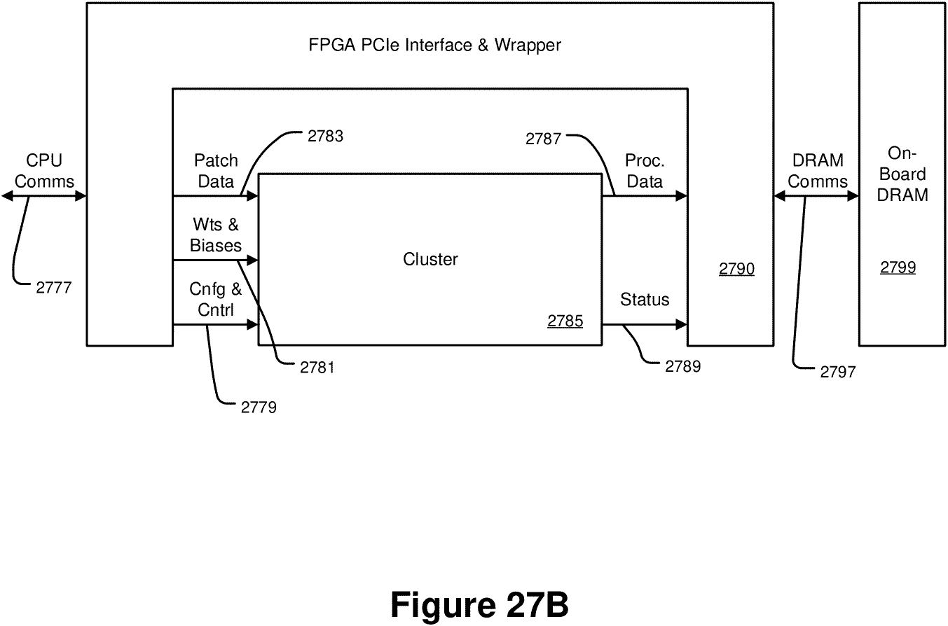

FIG. 27A is a simplified diagram showing aspects of the base calling operation, including functions of a runtime program (e.g., the runtime logic 2680) executed by a host processor. In this diagram, the output of image sensors from a flow cell are provided on lines 2700 to image processing threads 2701, which can perform processes on images such as alignment and arrangement in an array of sensor data for the individual tiles and resampling of images, and can be used by processes which calculate a tile cluster mask for each tile in the flow cell, which identifies pixels in the array of sensor data that correspond to clusters of genetic material on the corresponding tile of the flow cell. The outputs of the image processing threads 2701 are provided on lines 2702 to a dispatch logic 2703 in the CPU which routes the arrays of tile data to a data cache 2705 (e.g., SSD storage) on a high-speed bus 2704, or on high-speed bus 2706 to the neural network processor hardware 2707, such as the configurable processor 2646 of FIG. 26C, according to the state of the base calling operation. The processed and transformed images can be stored on the data cache 2705 for sensing cycles that were previously used. The neural network processor hardware 2707 returns classification data output by the neural network to the dispatch logic 2703, which passes the information to the data cache 2705, or on lines 2708 to base call and quality score threads 2709 that perform base call and quality score computations using the classification data, and can arrange the data in standard formats for base call reads. The outputs of the base call and quality score threads 2709 that perform base calling and quality score computations are provided on lines 2710 to threads 2711 that aggregate the base call reads, perform other operations such as data compression, and write the resulting base call outputs to specified destinations for utilization by the customers.

In some implementations, the host can include threads (not shown) that perform final processing of the output of the neural network processor hardware 2707 in support of the neural network. For example, the neural network processor hardware 2707 can provide outputs of classification data from a final layer of the multi-cluster neural network. The host processor can execute an output activation function, such as a softmax function, over the classification data to configure the data for use by the base call and quality score threads 2709. Also, the host processor can execute input operations (not shown), such as batch normalization of the tile data prior to input to the neural network processor hardware 2707.

FIG. 27B is a simplified diagram of a configuration of a configurable processor 2646 such as that of FIG. 26C. In FIG. 27B, the configurable processor 2646 comprises an FPGA with a plurality of high speed PCIe interfaces. The FPGA is configured with a wrapper 2790 which comprises the data flow logic 2697 described with reference to FIG. 26C. The wrapper 2790 manages the interface and coordination with a runtime program in the CPU across the CPU communication link 2777 and manages communication with the on-board DRAM 2799 (e.g., memory 2648A) via DRAM communication link 2797. The data flow logic 2697 in the wrapper 2790 provides patch data retrieved by traversing the arrays of tile data on the on-board DRAM 2799 for the number N cycles to a cluster 2785, and retrieves process data 2787 from the cluster 2785 for delivery back to the on-board DRAM 2799. The wrapper 2790 also manages transfer of data between the on-board DRAM 2799 and host memory, for both the input arrays of tile data, and for the output patches of classification data. The wrapper transfers patch data on line 2783 to the allocated cluster 2785. The wrapper provides trained parameters, such as weights and biases on line 2781 to the cluster 2785 retrieved from the on-board DRAM 2799. The wrapper provides configuration and control data on line 2779 to the cluster 2785 provided from, or generated in response to, the runtime program on the host via the CPU communication link 2777. The cluster can also provide status signals on line 2789 to the wrapper 2790, which are used in cooperation with control signals from the host to manage traversal of the arrays of tile data to provide spatially aligned patch data, and to execute the multi-cycle neural network over the patch data using the resources of the cluster 2785.

As mentioned above, there can be multiple clusters on a single configurable processor managed by the wrapper 2790 configured for executing on corresponding ones of multiple patches of the tile data. Each cluster can be configured to provide classification data for base calls in a subject sensing cycle using the tile data of multiple sensing cycles described herein.

In examples of the system, model data, including kernel data like filter weights and biases can be sent from the host CPU to the configurable processor, so that the model can be updated as a function of cycle number. A base calling operation can comprise, for a representative example, on the order of hundreds of sensing cycles. Base calling operation can include paired end reads in some implementations. For example, the model trained parameters may be updated once every 20 cycles (or other number of cycles), or according to update patterns implemented for particular systems and neural network models. In some implementations including paired end reads in which a sequence for a given string in a genetic cluster on a tile includes a first part extending from a first end down (or up) the string, and a second part extending from a second end up (or down) the string, the trained parameters can be updated on the transition from the first part to the second part.

In some examples, image data for multiple cycles of sensing data for a tile can be sent from the CPU to the wrapper 2790. The wrapper 2790 can optionally do some pre-processing and transformation of the sensing data and write the information to the on-board DRAM 2799. The input tile data for each sensing cycle can include arrays of sensor data including on the order of 4000 x 3000 pixels per sensing cycle per tile or more, with two features representing colors of two images of the tile, and one or two bytes per feature per pixel. For an implementation in which the number N is three sensing cycles to be used in each run of the multi-cycle neural network, the array of tile data for each run of the multi-cycle neural network can consume on the order of hundreds of megabytes per tile. In some implementations of the system, the tile data also includes an array of distance-from-cluster center (DFC) data, stored once per tile, or other type of metadata about the sensor data and the tiles.

In operation, when a multi-cycle cluster is available, the wrapper allocates a patch to the cluster. The wrapper fetches a next patch of tile data in the traversal of the tile and sends it to the allocated cluster along with appropriate control and configuration information. The cluster can be configured with enough memory on the configurable processor to hold a patch of data including patches from multiple cycles in some systems, that is being worked on in place, and a patch of data that is to be worked on when the current patch of processing is finished using a ping-pong buffer technique or raster scanning technique in various implementations.

When an allocated cluster completes its run of the neural network for the current patch and produces an output patch, it will signal the wrapper. The wrapper will read the output patch from the allocated cluster, or alternatively the allocated cluster will push the data out to the wrapper. Then the wrapper will assemble output patches for the processed tile in the on-board DRAM 2799. When the processing of the entire tile has been completed, and the output patches of data transferred to the DRAM, the wrapper sends the processed output array for the tile back to the host/CPU in a specified format. In some implementations, the on-board DRAM 2799 is managed by memory management logic in the wrapper 2790. The runtime program can control the sequencing operations to complete analysis of all the arrays of tile data for all the cycles in the run in a continuous flow to provide real-time analysis.

### Terminology and Additional Implementations

Base calling includes incorporation or attachment of a fluorescently-labeled tag with an analyte. The analyte can be a nucleotide or an oligonucleotide, and the tag can be for a particular nucleotide type (A, C, T, or G). Excitation light is directed toward the analyte having the tag, and the tag emits a detectable fluorescent signal or intensity emission. The intensity emission is indicative of photons emitted by the excited tag that is chemically attached to the analyte.

Throughout this application, including the claims, when phrases such as or similar to “images, image data, or image regions depicting intensity emissions of analytes and their surrounding background” are used, they refer to the intensity emissions of the tags attached to the analytes. A person skilled in the art will appreciate that the intensity emissions of the attached tags are representative of or equivalent to the intensity emissions of the analytes to which the tags are attached, and are therefore used interchangeably. Similarly, properties of the analytes refer to properties of the tags attached to the analytes or of the intensity emissions from the attached tags. For example, a center of an analyte refers to the center of the intensity emissions emitted by a tag attached to the analyte. In another example, the surrounding background of an analyte refers to the surrounding background of the intensity emissions emitted by a tag attached to the analyte.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The technology disclosed uses neural networks to improve the quality and quantity of nucleic acid sequence information that can be obtained from a nucleic acid sample such as a nucleic acid template or its complement, for instance, a DNA or RNA polynucleotide or other nucleic acid sample. Accordingly, certain implementations of the technology disclosed provide higher throughput polynucleotide sequencing, for instance, higher rates of collection of DNA or RNA sequence data, greater efficiency in sequence data collection, and/or lower costs of obtaining such sequence data, relative to previously available methodologies.

The technology disclosed uses neural networks to identify the center of a solid-phase nucleic acid cluster and to analyze optical signals that are generated during sequencing of such clusters, to discriminate unambiguously between adjacent, abutting or overlapping clusters in order to assign a sequencing signal to a single, discrete source cluster. These and related implementations thus permit retrieval of meaningful information, such as sequence data, from regions of high-density cluster arrays where useful information could not previously be obtained from such regions due to confounding effects of overlapping or very closely spaced adjacent clusters, including the effects of overlapping signals (e.g., as used in nucleic acid sequencing) emanating therefrom.

As described in greater detail below, in certain implementations there is provided a composition that comprises a solid support having immobilized thereto one or a plurality of nucleic acid clusters as provided herein. Each cluster comprises a plurality of immobilized nucleic acids of the same sequence and has an identifiable center having a detectable center label as provided herein, by which the identifiable center is distinguishable from immobilized nucleic acids in a surrounding region in the cluster. Also described herein are methods for making and using such clusters that have identifiable centers.

The presently disclosed implementations will find uses in numerous situations where advantages are obtained from the ability to identify, determine, annotate, record or otherwise assign the position of a substantially central location within a cluster, such as high-throughput nucleic acid sequencing, development of image analysis algorithms for assigning optical or other signals to discrete source clusters, and other applications where recognition of the center of an immobilized nucleic acid cluster is desirable and beneficial.

In certain implementations, the present invention contemplates methods that relate to high-throughput nucleic acid analysis such as nucleic acid sequence determination (e.g., “sequencing”). Exemplary high-throughput nucleic acid analyses include without limitation de novo sequencing, re-sequencing, whole genome sequencing, gene expression analysis, gene expression monitoring, epigenetic analysis, genome methylation analysis, allele specific primer extension (APSE), genetic diversity profiling, whole genome polymorphism discovery and analysis, single nucleotide polymorphism analysis, hybridization based sequence determination methods, and the like. One skilled in the art will appreciate that a variety of different nucleic acids can be analyzed using the methods and compositions of the present invention.

Although the implementations of the present invention are described in relation to nucleic acid sequencing, they are applicable in any field where image data acquired at different time points, spatial locations or other temporal or physical perspectives is analyzed. For example, the methods and systems described herein are useful in the fields of molecular and cell biology where image data from microarrays, biological specimens, cells, organisms and the like is acquired and at different time points or perspectives and analyzed. Images can be obtained using any number of techniques known in the art including, but not limited to, fluorescence microscopy, light microscopy, confocal microscopy, optical imaging, magnetic resonance imaging, tomography scanning or the like. As another example, the methods and systems described herein can be applied where image data obtained by surveillance, aerial or satellite imaging technologies and the like is acquired at different time points or perspectives and analyzed. The methods and systems are particularly useful for analyzing images obtained for a field of view in which the analytes being viewed remain in the same locations relative to each other in the field of view. The analytes may however have characteristics that differ in separate images, for example, the analytes may appear different in separate images of the field of view. For example, the analytes may appear different with regard to the color of a given analyte detected in different images, a change in the intensity of signal detected for a given analyte in different images, or even theappearance of a signal for a given analyte in one image and disappearance of the signal for the analyte in another image.

Examples described herein may be used in various biological or chemical processes and systems for academic or commercial analysis. More specifically, examples described herein may be used in various processes and systems where it is desired to detect an event, property, quality, or characteristic that is indicative of a designated reaction. For example, examples described herein include light detection devices, biosensors, and their components, as well as bioassay systems that operate with biosensors. In some examples, the devices, biosensors and systems may include a flow cell and one or more light sensors that are coupled together (removably or fixedly) in a substantially unitary structure.

The devices, biosensors and bioassay systems may be configured to perform a plurality of designated reactions that may be detected individually or collectively. The devices, biosensors and bioassay systems may be configured to perform numerous cycles in which the plurality of designated reactions occurs in parallel. For example, the devices, biosensors and bioassay systems may be used to sequence a dense array of DNA features through iterative cycles of enzymatic manipulation and light or image detection/acquisition. As such, the devices, biosensors and bioassay systems (e.g., via one or more cartridges) may include one or more microfluidic channel that delivers reagents or other reaction components in a reaction solution to a reaction site of the devices, biosensors and bioassay systems. In some examples, the reaction solution may be substantially acidic, such as comprising a pH of less than or equal to about 5, or less than or equal to about 4, or less than or equal to about 3. In some other examples, the reaction solution may be substantially alkaline/basic, such as comprising a pH of greater than or equal to about 8, or greater than or equal to about 9, or greater than or equal to about 10. As used herein, the term “acidity” and grammatical variants thereof refer to a pH value of less than about 7, and the terms “basicity,” “alkalinity” and grammatical variants thereof refer to a pH value of greater than about 7.

In some examples, the reaction sites are provided or spaced apart in a predetermined manner, such as in a uniform or repeating pattern. In some other examples, the reaction sites are randomly distributed. Each of the reaction sites may be associated with one or more light guides and one or more light sensors that detect light from the associated reaction site. In some examples, the reaction sites are located in reaction recesses or chambers, which may at least partially compartmentalize the designated reactions therein.

As used herein, a “designated reaction” includes a change in at least one of a chemical, electrical, physical, or optical property (or quality) of a chemical or biological substance of interest, such as an analyte-of-interest. In particular examples, a designated reaction is a positive binding event, such as incorporation of a fluorescently labeled biomolecule with an analyte-of-interest, for example. More generally, a designated reaction may be a chemical transformation, chemical change, or chemical interaction. A designated reaction may also be a change in electrical properties. In particular examples, a designated reaction includes the incorporation of a fluorescently-labeled molecule with an analyte. The analyte may be an oligonucleotide and the fluorescently-labeled molecule may be a nucleotide. A designated reaction may be detected when an excitation light is directed toward the oligonucleotide having the labeled nucleotide, and the fluorophore emits a detectable fluorescent signal. In alternative examples, the detected fluorescence is a result of chemiluminescence or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore, or decrease fluorescence by co-locating a quencher and fluorophore.

As used herein, a “reaction solution,” “reaction component” or “reactant” includes any substance that may be used to obtain at least one designated reaction. For example, potential reaction components include reagents, enzymes, samples, other biomolecules, and buffer solutions, for example. The reaction components may be delivered to a reaction site in a solution and/or immobilized at a reaction site. The reaction components may interact directly or indirectly with another substance, such as an analyte-of-interest immobilized at a reaction site. As noted above, the reaction solution may be substantially acidic (i.e., include a relatively high acidity) (e.g., comprising a pH of less than or equal to about 5, a pH less than or equal to about 4, or a pH less than or equal to about 3) or substantially alkaline/basic (i.e., include a relatively high alkalinity/basicity) (e.g., comprising a pH of greater than or equal to about 8, a pH of greater than or equal to about 9, or a pH of greater than or equal to about 10).

As used herein, the term “reaction site” is a localized region where at least one designated reaction may occur. A reaction site may include support surfaces of a reaction structure or substrate where a substance may be immobilized thereon. For example, a reaction site may include a surface of a reaction structure (which may be positioned in a channel of a flow cell) that has a reaction component thereon, such as a colony of nucleic acids thereon. In some such examples, the nucleic acids in the colony have the same sequence, being for example, clonal copies of a single stranded or double stranded template. However, in some examples a reaction site may contain only a single nucleic acid molecule, for example, in a single stranded or double stranded form.

A plurality of reaction sites may be randomly distributed along the reaction structure or arranged in a predetermined manner (e.g., side-by-side in a matrix, such as in microarrays). A reaction site can also include a reaction chamber or recess that at least partially defines a spatial region or volume configured to compartmentalize the designated reaction. As used herein, the term “reaction chamber” or “reaction recess” includes a defined spatial region of the support structure (which is often in fluid communication with a flow channel). A reaction recess may be at least partially separated from the surrounding environment other or spatial regions. For example, a plurality of reaction recesses may be separated from each other by shared walls, such as a detection surface. As a more specific example, the reaction recesses may be nanowells comprising an indent, pit, well, groove, cavity or depression defined by interior surfaces of a detection surface and have an opening or aperture (i.e., be open-sided) so that the nanowells can be in fluid communication with a flow channel.

In some examples, the reaction recesses of the reaction structure are sized and shaped relative to solids (including semi-solids) so that the solids may be inserted, fully or partially, therein. For example, the reaction recesses may be sized and shaped to accommodate a capture bead. The capture bead may have clonally amplified DNA or other substances thereon. Alternatively, the reaction recesses may be sized and shaped to receive an approximate number of beads or solid substrates. As another example, the reaction recesses may be filled with a porous gel or substance that is configured to control diffusion or filter fluids or solutions that may flow into the reaction recesses.

In some examples, light sensors (e.g., photodiodes) are associated with corresponding reaction sites. A light sensor that is associated with a reaction site is configured to detect light emissions from the associated reaction site via at least one light guide when a designated reaction has occurred at the associated reaction site. In some cases, a plurality of light sensors (e.g. several pixels of a light detection or camera device) may be associated with a single reaction site. In other cases, a single light sensor (e.g. a single pixel) may be associated with a single reaction site or with a group of reaction sites. The light sensor, the reaction site, and other features of the biosensor may be configured so that at least some of the light is directly detected by the light sensor without being reflected.

As used herein, a “biological or chemical substance” includes biomolecules, samples-of-interest, analytes-of-interest, and other chemical compound(s). A biological or chemical substance may be used to detect, identify, or analyze other chemical compound(s), or function as intermediaries to study or analyze other chemical compound(s). In particular examples, the biological or chemical substances include a biomolecule. As used herein, a “biomolecule” includes at least one of a biopolymer, nucleoside, nucleic acid, polynucleotide, oligonucleotide, protein, enzyme, polypeptide, antibody, antigen, ligand, receptor, polysaccharide, carbohydrate, polyphosphate, cell, tissue, organism, or fragment thereof or any other biologically active chemical compound(s) such as analogs or mimetics of the aforementioned species. In a further example, a biological or chemical substance or a biomolecule includes an enzyme or reagent used in a coupled reaction to detect the product of another reaction such as an enzyme or reagent, such as an enzyme or reagent used to detect pyrophosphate in a pyrosequencing reaction. Enzymes and reagents useful for pyrophosphate detection are described, for example, in U.S. Pat. Publication No. 2005/0244870A1, which is incorporated by reference in its entirety.

Biomolecules, samples, and biological or chemical substances may be naturally occurring or synthetic and may be suspended in a solution or mixture within a reaction recess or region. Biomolecules, samples, and biological or chemical substances may also be bound to a solid phase or gel material. Biomolecules, samples, and biological or chemical substances may also include a pharmaceutical composition. In some cases, biomolecules, samples, and biological or chemical substances of interest may be referred to as targets, probes, or analytes.

As used herein, a “biosensor” includes a device that includes a reaction structure with a plurality of reaction sites that is configured to detect designated reactions that occur at or proximate to the reaction sites. A biosensor may include a solid-state light detection or “imaging” device (e.g., CCD or CMOS light detection device) and, optionally, a flow cell mounted thereto. The flow cell may include at least one flow channel that is in fluid communication with the reaction sites. As one specific example, the biosensor is configured to fluidically and electrically couple to a bioassay system. The bioassay system may deliver a reaction solution to the reaction sites according to a predetermined protocol (e.g., sequencing-by-synthesis) and perform a plurality of imaging events. For example, the bioassay system may direct reaction solutions to flow along the reaction sites. At least one of the reaction solutions may include four types of nucleotides having the same or different fluorescent labels. The nucleotides may bind to the reaction sites, such as to corresponding oligonucleotides at the reaction sites. The bioassay system may then illuminate the reaction sites using an excitation light source (e.g., solid-state light sources, such as light-emitting diodes (LEDs)). The excitation light may have a predetermined wavelength or wavelengths, including a range of wavelengths. The fluorescent labels excited by the incident excitation light may provide emission signals (e.g., light of a wavelength or wavelengths that differ from the excitation light and, potentially, each other) that may be detected by the light sensors.

As used herein, the term “immobilized,” when used with respect to a biomolecule or biological or chemical substance, includes substantially attaching the biomolecule or biological or chemical substance at a molecular level to a surface, such as to a detection surface of a light detection device or reaction structure. For example, a biomolecule or biological or chemical substance may be immobilized to a surface of the reaction structure using adsorption techniques including non-covalent interactions (e.g., electrostatic forces, van der Waals, and dehydration of hydrophobic interfaces) and covalent binding techniques where functional groups or linkers facilitate attaching the biomolecules to the surface. Immobilizing biomolecules or biological or chemical substances to the surface may be based upon the properties of the surface, the liquid medium carrying the biomolecule or biological or chemical substance, and the properties of the biomolecules or biological or chemical substances themselves. In some cases, the surface may be functionalized (e.g., chemically or physically modified) to facilitate immobilizing the biomolecules (or biological or chemical substances) to the surface.

In some examples, nucleic acids can be immobilized to the reaction structure, such as to surfaces of reaction recesses thereof. In particular examples, the devices, biosensors, bioassay systems and methods described herein may include the use of natural nucleotides and also enzymes that are configured to interact with the natural nucleotides. Natural nucleotides include, for example, ribonucleotides or deoxyribonucleotides. Natural nucleotides can be in the mono-, di-, or tri-phosphate form and can have a base selected from adenine (A), Thymine (T), uracil (U), guanine (G) or cytosine (C). It will be understood, however, that non-natural nucleotides, modified nucleotides or analogs of the aforementioned nucleotides can be used.

As noted above, a biomolecule or biological or chemical substance may be immobilized at a reaction site in a reaction recess of a reaction structure. Such a biomolecule or biological substance may be physically held or immobilized within the reaction recesses through an interference fit, adhesion, covalent bond, or entrapment. Examples of items or solids that may be disposed within the reaction recesses include polymer beads, pellets, agarose gel, powders, quantum dots, or other solids that may be compressed and/or held within the reaction chamber. In certain implementations, the reaction recesses may be coated or filled with a hydrogel layer capable of covalently binding DNA oligonucleotides. In particular examples, a nucleic acid superstructure, such as a DNA ball, can be disposed in or at a reaction recess, for example, by attachment to an interior surface of the reaction recess or by residence in a liquid within the reaction recess. A DNA ball or other nucleic acid superstructure can be performed and then disposed in or at a reaction recess. Alternatively, a DNA ball can be synthesized in situ at a reaction recess. A substance that is immobilized in a reaction recess can be in a solid, liquid, or gaseous state.

As used herein, the term “analyte” is intended to mean a point or area in a pattern that can be distinguished from other points or areas according to relative location. An individual analyte can include one or more molecules of a particular type. For example, an analyte can include a single target nucleic acid molecule having a particular sequence or an analyte can include several nucleic acid molecules having the same sequence (and/or complementary sequence, thereof). Different molecules that are at different analytes of a pattern can be differentiated from each other according to the locations of the analytes in the pattern. Example analytes include without limitation, wells in a substrate, beads (or other particles) in or on a substrate, projections from a substrate, ridges on a substrate, pads of gel material on a substrate, or channels in a substrate.

Any of a variety of target analytes that are to be detected, characterized, or identified can be used in an apparatus, system or method set forth herein. Exemplary analytes include, but are not limited to, nucleic acids (e.g., DNA, RNA or analogs thereof), proteins, polysaccharides, cells, antibodies, epitopes, receptors, ligands, enzymes (e.g. kinases, phosphatases or polymerases), small molecule drug candidates, cells, viruses, organisms, or the like.

The terms “analyte”, “nucleic acid”, “nucleic acid molecule”, and “polynucleotide” are used interchangeably herein. In various implementations, nucleic acids may be used as templates as provided herein (e.g., a nucleic acid template, or a nucleic acid complement that is complementary to a nucleic acid nucleic acid template) for particular types of nucleic acid analysis, including but not limited to nucleic acid amplification, nucleic acid expression analysis, and/or nucleic acid sequence determination or suitable combinations thereof. Nucleic acids in certain implementations include, for instance, linear polymers of deoxyribonucleotides in 3′-5′ phosphodiester or other linkages, such as deoxyribonucleic acids (DNA), for example, single- and double-stranded DNA, genomic DNA, copy DNA or complementary DNA (cDNA), recombinant DNA, or any form of synthetic or modified DNA. In other implementations, nucleic acids include for instance, linear polymers of ribonucleotides in 3′-5′ phosphodiester or other linkages such as ribonucleic acids (RNA), for example, single- and double-stranded RNA, messenger (mRNA), copy RNA or complementary RNA (cRNA), alternatively spliced mRNA, ribosomal RNA, small nucleolar RNA (snoRNA), microRNAs (miRNA), small interfering RNAs (sRNA), piwi RNAs (piRNA), or any form of synthetic or modified RNA. Nucleic acids used in the compositions and methods of the present invention may vary in length and may be intact or full-length molecules or fragments or smaller parts of larger nucleic acid molecules. In particular implementations, a nucleic acid may have one or more detectable labels, as described elsewhere herein.

The terms “analyte,” “cluster,” “nucleic acid cluster,” “nucleic acid colony,” and “DNA cluster” are used interchangeably and refer to a plurality of copies of a nucleic acid template and/or complements thereof attached to a solid support. Typically and in certain preferred implementations, the nucleic acid cluster comprises a plurality of copies of template nucleic acid and/or complements thereof, attached via their 5′ termini to the solid support. The copies of nucleic acid strands making up the nucleic acid clusters may be in a single or double stranded form. Copies of a nucleic acid template that are present in a cluster can have nucleotides at corresponding positions that differ from each other, for example, due to presence of a label moiety. The corresponding positions can also contain analog structures having different chemical structure but similar Watson-Crick base-pairing properties, such as is the case for uracil and thymine.

Colonies of nucleic acids can also be referred to as “nucleic acid clusters”. Nucleic acid colonies can optionally be created by cluster amplification or bridge amplification techniques as set forth in further detail elsewhere herein. Multiple repeats of a target sequence can be present in a single nucleic acid molecule, such as a concatamer created using a rolling circle amplification procedure.

The nucleic acid clusters of the invention can have different shapes, sizes and densities depending on the conditions used. For example, clusters can have a shape that is substantially round, multi-sided, donut-shaped or ring-shaped. The diameter of a nucleic acid cluster can be designed to be from about 0.2 µm to about 6 µm, about 0.3 µm to about 4 µm, about 0.4 µm to about 3 µm, about 0.5 µm to about 2 µm, about 0.75 µm to about 1.5 µm, or any intervening diameter. In a particular implementation, the diameter of a nucleic acid cluster is about 0.5 µm, about 1 µm, about 1.5 µm, about 2 µm, about 2.5 µm, about 3 µm, about 4 µm, about 5 µm, or about 6 µm. The diameter of a nucleic acid cluster may be influenced by a number of parameters, including, but not limited to the number of amplification cycles performed in producing the cluster, the length of the nucleic acid template or the density of primers attached to the surface upon which clusters are formed. The density of nucleic acid clusters can be designed to typically be in the range of 0.1/mm², 1/mm², 10/mm², 100/mm², 1,000/mm², 10,000/mm² to 100,000/mm². The present invention further contemplates, in part, higher density nucleic acid clusters, for example, 100,000/mm² to 1,000,000/mm² and 1,000,000/mm² to 10,000,000/mm².

As used herein, an “analyte” is an area of interest within a specimen or field of view. When used in connection with microarray devices or other molecular analytical devices, an analyte refers to the area occupied by similar or identical molecules. For example, an analyte can be an amplified oligonucleotide or any other group of a polynucleotide or polypeptide with a same or similar sequence. In other implementations, an analyte can be any element or group of elements that occupy a physical area on a specimen. For example, an analyte could be a parcel of land, a body of water or the like. When an analyte is imaged, each analyte will have some area. Thus, in many implementations, an analyte is not merely one pixel.

The distances between analytes can be described in any number of ways. In some implementations, the distances between analytes can be described from the center of one analyte to the center of another analyte. In other implementations, the distances can be described from the edge of one analyte to the edge of another analyte, or between the outer-most identifiable points of each analyte. The edge of an analyte can be described as the theoretical or actual physical boundary on a chip, or some point inside the boundary of the analyte. In other implementations, the distances can be described in relation to a fixed point on the specimen or in the image of the specimen.

Generally several implementations will be described herein with respect to a method of analysis. It will be understood that systems are also provided for carrying out the methods in an automated or semi-automated way. Accordingly, this disclosure provides neural network-based template generation and base calling systems, wherein the systems can include a processor; a storage device; and a program for image analysis, the program including instructions for carrying out one or more of the methods set forth herein. Accordingly, the methods set forth herein can be carried out on a computer, for example, having components set forth herein or otherwise known in the art.

The methods and systems set forth herein are useful for analyzing any of a variety of objects. Particularly useful objects are solid supports or solid-phase surfaces with attached analytes. The methods and systems set forth herein provide advantages when used with objects having a repeating pattern of analytes in an xy plane. An example is a microarray having an attached collection of cells, viruses, nucleic acids, proteins, antibodies, carbohydrates, small molecules (such as drug candidates), biologically active molecules or other analytes of interest.

An increasing number of applications have been developed for arrays with analytes having biological molecules such as nucleic acids and polypeptides. Such microarrays typically include deoxyribonucleic acid (DNA) or ribonucleic acid (RNA) probes. These are specific for nucleotide sequences present in humans and other organisms. In certain applications, for example, individual DNA or RNA probes can be attached at individual analytes of an array. A test sample, such as from a known person or organism, can be exposed to the array, such that target nucleic acids (e.g., gene fragments, mRNA, or amplicons thereof) hybridize to complementary probes at respective analytes in the array. The probes can be labeled in a target specific process (e.g., due to labels present on the target nucleic acids or due to enzymatic labeling of the probes or targets that are present in hybridized form at the analytes). The array can then be examined by scanning specific frequencies of light over the analytes to identify which target nucleic acids are present in the sample.

Biological microarrays may be used for genetic sequencing and similar applications. In general, genetic sequencing comprises determining the order of nucleotides in a length of target nucleic acid, such as a fragment of DNA or RNA. Relatively short sequences are typically sequenced at each analyte, and the resulting sequence information may be used in various bioinformatics methods to logically fit the sequence fragments together so as to reliably determine the sequence of much more extensive lengths of genetic material from which the fragments were derived. Automated, computer-based algorithms for characteristic fragments have been developed, and have been used more recently in genome mapping, identification of genes and their function, and so forth. Microarrays are particularly useful for characterizing genomic content because a large number of variants are present and this supplants the alternative of performing many experiments on individual probes and targets. The microarray is an ideal format for performing such investigations in a practical manner.

Any of a variety of analyte arrays (also referred to as “microarrays”) known in the art can be used in a method or system set forth herein. A typical array contains analytes, each having an individual probe or a population of probes. In the latter case, the population of probes at each analyte is typically homogenous having a single species of probe. For example, in the case of a nucleic acid array, each analyte can have multiple nucleic acid molecules each having a common sequence. However, in some implementations the populations at each analyte of an array can be heterogeneous. Similarly, protein arrays can have analytes with a single protein or a population of proteins typically, but not always, having the same amino acid sequence. The probes can be attached to the surface of an array for example, via covalent linkage of the probes to the surface or via non-covalent interaction(s) of the probes with the surface. In some implementations, probes, such as nucleic acid molecules, can be attached to a surface via a gel layer as described, for example, in U.S. Pat. Application Ser. No. 13/784,368 and U.S. Pat. App. Pub. No. 2011/0059865A1, each of which is incorporated herein by reference.

Example arrays include, without limitation, a BeadChip Array available from Illumina, Inc. (San Diego, Calif.) or others such as those where probes are attached to beads that are present on a surface (e.g. beads in wells on a surface) such as those described in U.S. Pat. No. 6,266,459; 6,355,431; 6,770,441; 6,859,570; or 7,622,294; or PCT Publication No. WO 00/63437, each of which is incorporated herein by reference. Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies. A spotted microarray can also be used in a method or system according to some implementations of the present disclosure. An example spotted microarray is a CodeLink™ Array available from Amersham Biosciences. Another microarray that is useful is one that is manufactured using inkjet printing methods such as SurePrint™ Technology available from Agilent Technologies.

Other useful arrays include those that are used in nucleic acid sequencing applications. For example, arrays having amplicons of genomic fragments (often referred to as clusters) are particularly useful such as those described in Bentley et al., Nature 456:53-59 (2008), WO 04/018497; WO 91/06678; WO 07/123744; U.S. Pat. No. 7,329,492; 7,211,414; 7,315,019; 7,405,281, or 7,057,026; or U.S. Pat. App. Pub. No. 2008/0108082A1, each of which is incorporated herein by reference. Another type of array that is useful for nucleic acid sequencing is an array of particles produced from an emulsion PCR technique. Examples are described in Dressman et al., Proc. Natl. Acad. Sci. USA 100:8817-8822 (2003), WO 05/010145, U.S. Pat. App. Pub. No. 2005/0130173 or U.S. Pat. App. Pub. No. 2005/0064460, each of which is incorporated herein by reference in its entirety.

Arrays used for nucleic acid sequencing often have random spatial patterns of nucleic acid analytes. For example, HiSeq or MiSeq sequencing platforms available from Illumina Inc. (San Diego, Calif.) utilize flow cells upon which nucleic acid arrays are formed by random seeding followed by bridge amplification. However, patterned arrays can also be used for nucleic acid sequencing or other analytical applications. Example patterned arrays, methods for their manufacture and methods for their use are set forth in U.S. Ser. No. 13/787,396; U.S. Ser. No. 13/783,043; U.S. Ser. No. 13/784,368; U.S. Pat. App. Pub. No. 2013/0116153A1; and U.S. Pat. App. Pub. No. 2012/0316086A1, each of which is incorporated herein by reference. The analytes of such patterned arrays can be used to capture a single nucleic acid template molecule to seed subsequent formation of a homogenous colony, for example, via bridge amplification. Such patterned arrays are particularly useful for nucleic acid sequencing applications.

The size of an analyte on an array (or other object used in a method or system herein) can be selected to suit a particular application. For example, in some implementations, an analyte of an array can have a size that accommodates only a single nucleic acid molecule. A surface having a plurality of analytes in this size range is useful for constructing an array of molecules for detection at single molecule resolution. Analytes in this size range are also useful for use in arrays having analytes that each contain a colony of nucleic acid molecules. Thus, the analytes of an array can each have an area that is no larger than about 1 mm², no larger than about 500 µm², no larger than about 100 µm², no larger than about 10 µm², no larger than about 1 µm², no larger than about 500 nm², or no larger than about 100 nm², no larger than about 10 nm², no larger than about 5 nm², or no larger than about 1 nm². Alternatively or additionally, the analytes of an array will be no smaller than about 1 mm², no smaller than about 500 µm², no smaller than about 100 µm², no smaller than about 10 µm², no smaller than about 1 µm², no smaller than about 500 nm², no smaller than about 100 nm², no smaller than about 10 nm², no smaller than about 5 nm², or no smaller than about 1 nm². Indeed, an analyte can have a size that is in a range between an upper and lower limit selected from those exemplified above. Although several size ranges for analytes of a surface have been exemplified with respect to nucleic acids and on the scale of nucleic acids, it will be understood that analytes in these size ranges can be used for applications that do not include nucleic acids. It will be further understood that the size of the analytes need not necessarily be confined to a scale used for nucleic acid applications.

For implementations that include an object having a plurality of analytes, such as an array of analytes, the analytes can be discrete, being separated with spaces between each other. An array useful in the invention can have analytes that are separated by edge to edge distance of at most 100 µm, 50 µm, 10 µm, 5 µm, 1 µm, 0.5 µm, or less. Alternatively or additionally, an array can have analytes that are separated by an edge to edge distance of at least 0.5 µm, 1 µm, 5 µm, 10 µm, 50 µm, 100 µm, or more. These ranges can apply to the average edge to edge spacing for analytes as well as to the minimum or maximum spacing.

In some implementations the analytes of an array need not be discrete and instead neighboring analytes can abut each other. Whether or not the analytes are discrete, the size of the analytes and/or pitch of the analytes can vary such that arrays can have a desired density. For example, the average analyte pitch in a regular pattern can be at most 100 µm, 50 µm, 10 µm, 5 µm, 1 µm, 0.5 µm, or less. Alternatively or additionally, the average analyte pitch in a regular pattern can be at least 0.5 µm, 1 µm, 5 µm, 10 µm, 50 µm, 100 µm, or more. These ranges can apply to the maximum or minimum pitch for a regular pattern as well. For example, the maximum analyte pitch for a regular pattern can be at most 100 µm, 50 µm, 10 µm, 5 µm, 1 µm, 0.5 µm, or less; and/or the minimum analyte pitch in a regular pattern can be at least 0.5 µm, 1 µm, 5 µm, 10 µm, 50 µm, 100 µm, or more.

The density of analytes in an array can also be understood in terms of the number of analytes present per unit area. For example, the average density of analytes for an array can be at least about 1×10³ analytes/mm², 1×10⁴ analytes/mm², 1×10⁵ analytes/mm², 1×10⁶ analytes/mm², 1×10⁷ analytes/mm², 1×10⁸ analytes/mm², or 1×10⁹ analytes/mm², or higher. Alternatively or additionally the average density of analytes for an array can be at most about 1×10⁹ analytes/mm², 1×10⁸ analytes/mm², 1×10⁷ analytes/mm², 1×10⁶ analytes/mm², 1×10⁵ analytes/mm², 1×10⁴ analytes/mm², or 1×10³ analytes/mm², or less.

The above ranges can apply to all or part of a regular pattern including, for example, all or part of an array of analytes.

The analytes in a pattern can have any of a variety of shapes. For example, when observed in a two dimensional plane, such as on the surface of an array, the analytes can appear rounded, circular, oval, rectangular, square, symmetric, asymmetric, triangular, polygonal, or the like. The analytes can be arranged in a regular repeating pattern including, for example, a hexagonal or rectilinear pattern. A pattern can be selected to achieve a desired level of packing. For example, round analytes are optimally packed in a hexagonal arrangement. Of course other packing arrangements can also be used for round analytes and vice versa.

A pattern can be characterized in terms of the number of analytes that are present in a subset that forms the smallest geometric unit of the pattern. The subset can include, for example, at least about 2, 3, 4, 5, 6, 10 or more analytes. Depending upon the size and density of the analytes the geometric unit can occupy an area of less than 1 mm², 500 µm², 100 µm², 50 µm², 10 µm², 1 µm², 500 nm², 100 nm², 50 nm², 10 nm², or less. Alternatively or additionally, the geometric unit can occupy an area of greater than 10 nm², 50 nm², 100 nm², 500 ₙₘ², 1 µm², 10 µm², 50 µm², 100 µm², 500 µm², 1 mm², or more. Characteristics of the analytes in a geometric unit, such as shape, size, pitch and the like, can be selected from those set forth herein more generally with regard to analytes in an array or pattern.

An array having a regular pattern of analytes can be ordered with respect to the relative locations of the analytes but random with respect to one or more other characteristic of each analyte. For example, in the case of a nucleic acid array, the nuclei acid analytes can be ordered with respect to their relative locations but random with respect to one’s knowledge of the sequence for the nucleic acid species present at any particular analyte. As a more specific example, nucleic acid arrays formed by seeding a repeating pattern of analytes with template nucleic acids and amplifying the template at each analyte to form copies of the template at the analyte (e.g., via cluster amplification or bridge amplification) will have a regular pattern of nucleic acid analytes but will be random with regard to the distribution of sequences of the nucleic acids across the array. Thus, detection of the presence of nucleic acid material generally on the array can yield a repeating pattern of analytes, whereas sequence-specific detection can yield non-repeating distribution of signals across the array.

It will be understood that the description herein of patterns, order, randomness and the like pertain not only to analytes on objects, such as analytes on arrays, but also to analytes in images. As such, patterns, order, randomness and the like can be present in any of a variety of formats that are used to store, manipulate or communicate image data including, but not limited to, a computer readable medium or computer component such as a graphical user interface or other output device.

As used herein, the term “image” is intended to mean a representation of all or part of an object. The representation can be an optically detected reproduction. For example, an image can be obtained from fluorescent, luminescent, scatter, or absorption signals. The part of the object that is present in an image can be the surface or other xy plane of the object. Typically, an image is a 2 dimensional representation, but in some cases information in the image can be derived from 3 or more dimensions. An image need not include optically detected signals. Non-optical signals can be present instead. An image can be provided in a computer readable format or medium such as one or more of those set forth elsewhere herein.

As used herein, “image” refers to a reproduction or representation of at least a portion of a specimen or other object. In some implementations, the reproduction is an optical reproduction, for example, produced by a camera or other optical detector. The reproduction can be a non-optical reproduction, for example, a representation of electrical signals obtained from an array of nanopore analytes or a representation of electrical signals obtained from an ion-sensitive CMOS detector. In particular implementations non-optical reproductions can be excluded from a method or apparatus set forth herein. An image can have a resolution capable of distinguishing analytes of a specimen that are present at any of a variety of spacings including, for example, those that are separated by less than 100 µm, 50 µm, 10 µm, 5 µm, 1 µm or 0.5 µm.

As used herein, “acquiring”, “acquisition” and like terms refer to any part of the process of obtaining an image file. In some implementations, data acquisition can include generating an image of a specimen, looking for a signal in a specimen, instructing a detection device to look for or generate an image of a signal, giving instructions for further analysis or transformation of an image file, and any number of transformations or manipulations of an image file.

As used herein, the term “template” refers to a representation of the location or relation between signals or analytes. Thus, in some implementations, a template is a physical grid with a representation of signals corresponding to analytes in a specimen. In some implementations, a template can be a chart, table, text file or other computer file indicative of locations corresponding to analytes. In implementations presented herein, a template is generated in order to track the location of analytes of a specimen across a set of images of the specimen captured at different reference points. For example, a template could be a set of x,y coordinates or a set of values that describe the direction and/or distance of one analyte with respect to another analyte.

As used herein, the term “specimen” can refer to an object or area of an object of which an image is captured. For example, in implementations where images are taken of the surface of the earth, a parcel of land can be a specimen. In other implementations where the analysis of biological molecules is performed in a flow cell, the flow cell may be divided into any number of subdivisions, each of which may be a specimen. For example, a flow cell may be divided into various flow channels or lanes, and each lane can be further divided into 2, 3, 4, 5, 6, 7, 8, 9, 10, 20, 30, 40, 50, 60 70, 80, 90, 100, 110, 120, 140, 160, 180, 200, 400, 600, 800, 1000 or more separate regions that are imaged. One example of a flow cell has 8 lanes, with each lane divided into 120 specimens or tiles. In another implementation, a specimen may be made up of a plurality of tiles or even an entire flow cell. Thus, the image of each specimen can represent a region of a larger surface that is imaged.

It will be appreciated that references to ranges and sequential number lists described herein include not only the enumerated number but all real numbers between the enumerated numbers.

As used herein, a “reference point” refers to any temporal or physical distinction between images. In a preferred implementation, a reference point is a time point. In a more preferred implementation, a reference point is a time point or cycle during a sequencing reaction. However, the term “reference point” can include other aspects that distinguish or separate images, such as angle, rotational, temporal, or other aspects that can distinguish or separate images.

As used herein, a “subset of images” refers to a group of images within a set. For example, a subset may contain 1, 2, 3, 4, 6, 8, 10, 12, 14, 16, 18, 20, 30, 40, 50, 60 or any number of images selected from a set of images. In particular implementations, a subset may contain no more than 1, 2, 3, 4, 6, 8, 10, 12, 14, 16, 18, 20, 30, 40, 50, 60 or any number of images selected from a set of images. In a preferred implementation, images are obtained from one or more sequencing cycles with four images correlated to each cycle. Thus, for example, a subset could be a group of 16 images obtained through four cycles.

A base refers to a nucleotide base or nucleotide, A (adenine), C (cytosine), T (thymine), or G (guanine). This application uses “base(s)” and “nucleotide(s)” interchangeably.

The term “chromosome” refers to the heredity-bearing gene carrier of a living cell, which is derived from chromatin strands comprising DNA and protein components (especially histones). The conventional internationally recognized individual human genome chromosome numbering system is employed herein.

The term “site” refers to a unique position (e.g., chromosome ID, chromosome position and orientation) on a reference genome. In some implementations, a site may be a residue, a sequence tag, or a segment’s position on a sequence. The term “locus” may be used to refer to the specific location of a nucleic acid sequence or polymorphism on a reference chromosome.

The term “sample” herein refers to a sample, typically derived from a biological fluid, cell, tissue, organ, or organism containing a nucleic acid or a mixture of nucleic acids containing at least one nucleic acid sequence that is to be sequenced and/or phased. Such samples include, but are not limited to sputum/oral fluid, amniotic fluid, blood, a blood fraction, fine needle biopsy samples (e.g., surgical biopsy, fine needle biopsy, etc.), urine, peritoneal fluid, pleural fluid, tissue explant, organ culture and any other tissue or cell preparation, or fraction or derivative thereof or isolated therefrom. Although the sample is often taken from a human subject (e.g., patient), samples can be taken from any organism having chromosomes, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc.

The term “sequence” includes or represents a strand of nucleotides coupled to each other. The nucleotides may be based on DNA or RNA. It should be understood that one sequence may include multiple sub-sequences. For example, a single sequence (e.g., of a PCR amplicon) may have 350 nucleotides. The sample read may include multiple sub-sequences within these 350 nucleotides. For instance, the sample read may include first and second flanking subsequences having, for example, 20-50 nucleotides. The first and second flanking sub-sequences may be located on either side of a repetitive segment having a corresponding sub-sequence (e.g., 40-100 nucleotides). Each of the flanking sub-sequences may include (or include portions of) a primer sub-sequence (e.g., 10-30 nucleotides). For ease of reading, the term “sub-sequence” will be referred to as “sequence,” but it is understood that two sequences are not necessarily separate from each other on a common strand. To differentiate the various sequences described herein, the sequences may be given different labels (e.g., target sequence, primer sequence, flanking sequence, reference sequence, and the like). Other terms, such as “allele,” may be given different labels to differentiate between like objects. The application uses “read(s)” and “sequence read(s)” interchangeably.

The term “paired-end sequencing” refers to sequencing methods that sequence both ends of a target fragment. Paired-end sequencing may facilitate detection of genomic rearrangements and repetitive segments, as well as gene fusions and novel transcripts. Methodology for paired-end sequencing are described in PCT publication WO07010252, PCT application Serial No. PCTGB2007/003798 and U.S. Pat. Application Publication US 2009/0088327, each of which is incorporated by reference herein. In one example, a series of operations may be performed as follows; (a) generate clusters of nucleic acids; (b) linearize the nucleic acids; (c) hybridize a first sequencing primer and carry out repeated cycles of extension, scanning and deblocking, as set forth above; (d) “invert” the target nucleic acids on the flow cell surface by synthesizing a complimentary copy; (e) linearize the resynthesized strand; and (f) hybridize a second sequencing primer and carry out repeated cycles of extension, scanning and deblocking, as set forth above. The inversion operation can be carried out be delivering reagents as set forth above for a single cycle of bridge amplification.

The term “reference genome” or “reference sequence” refers to any particular known genome sequence, whether partial or complete, of any organism which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences. A genome includes both the genes and the noncoding sequences of the DNA. The reference sequence may be larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference genome sequence is that of a full length human genome. In another example, the reference genome sequence is limited to a specific human chromosome such as chromosome 13. In some implementations, a reference chromosome is a chromosome sequence from human genome version hg19. Such sequences may be referred to as chromosome reference sequences, although the term reference genome is intended to cover such sequences. Other examples of reference sequences include genomes of other species, as well as chromosomes, sub-chromosomal regions (such as strands), etc., of any species. In various implementations, the reference genome is a consensus sequence or other combination derived from multiple individuals. However, in certain applications, the reference sequence may be taken from a particular individual. In other implementations, the “genome” also covers so-called “graph genomes”, which use a particular storage format and representation of the genome sequence. In one implementation, graph genomes store data in a linear file. In another implementation, the graph genomes refer to a representation where alternative sequences (e.g., different copies of a chromosome with small differences) are stored as different paths in a graph. Additional information regarding graph genome implementations can be found in https://www.biorxiv.org/content/biorxiv/early/2018/03/20/194530.full.pdf, the content of which is hereby incorporated herein by reference in its entirety.

The term “read” refer to a collection of sequence data that describes a fragment of a nucleotide sample or reference. The term “read” may refer to a sample read and/or a reference read. Typically, though not necessarily, a read represents a short sequence of contiguous base pairs in the sample or reference. The read may be represented symbolically by the base pair sequence (in ACTG) of the sample or reference fragment. It may be stored in a memory device and processed as appropriate to determine whether the read matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

Next-generation sequencing methods include, for example, sequencing by synthesis technology (Illumina), pyrosequencing (454), ion semiconductor technology (Ion Torrent sequencing), single-molecule real-time sequencing and sequencing by ligation (SOLiD sequencing). Depending on the sequencing methods, the length of each read may vary from about 30 bp to more than 10,000 bp. For example, the DNA sequencing method using SOLiD sequencer generates nucleic acid reads of about 50 bp. For another example, Ion Torrent Sequencing generates nucleic acid reads of up to 400 bp and 454 pyrosequencing generates nucleic acid reads of about 700 bp. For yet another example, single-molecule real-time sequencing methods may generate reads of 10,000 bp to 15,000 bp. Therefore, in certain implementations, the nucleic acid sequence reads have a length of 30-100 bp, 50-200 bp, or 50-400 bp.

The terms “sample read”, “sample sequence” or “sample fragment” refer to sequence data for a genomic sequence of interest from a sample. For example, the sample read comprises sequence data from a PCR amplicon having a forward and reverse primer sequence. The sequence data can be obtained from any select sequence methodology. The sample read can be, for example, from a sequencing-by-synthesis (SBS) reaction, a sequencing-by-ligation reaction, or any other suitable sequencing methodology for which it is desired to determine the length and/or identity of a repetitive element. The sample read can be a consensus (e.g., averaged or weighted) sequence derived from multiple sample reads. In certain implementations, providing a reference sequence comprises identifying a locus-of-interest based upon the primer sequence of the PCR amplicon.

The term “raw fragment” refers to sequence data for a portion of a genomic sequence of interest that at least partially overlaps a designated position or secondary position of interest within a sample read or sample fragment. Non-limiting examples of raw fragments include a duplex stitched fragment, a simplex stitched fragment, a duplex un-stitched fragment and a simplex un-stitched fragment. The term “raw” is used to indicate that the raw fragment includes sequence data having some relation to the sequence data in a sample read, regardless of whether the raw fragment exhibits a supporting variant that corresponds to and authenticates or confirms a potential variant in a sample read. The term “raw fragment” does not indicate that the fragment necessarily includes a supporting variant that validates a variant call in a sample read. For example, when a sample read is determined by a variant call application to exhibit a first variant, the variant call application may determine that one or more raw fragments lack a corresponding type of “supporting” variant that may otherwise be expected to occur given the variant in the sample read.

The terms “mapping”, “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain implementations, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

The term “indel” refers to the insertion and/or the deletion of bases in the DNA of an organism. A micro-indel represents an indel that results in a net change of 1 to 50 nucleotides. In coding regions of the genome, unless the length of an indel is a multiple of 3, it will produce a frameshift mutation. Indels can be contrasted with point mutations. An indel inserts and deletes nucleotides from a sequence, while a point mutation is a form of substitution that replaces one of the nucleotides without changing the overall number in the DNA. Indels can also be contrasted with a Tandem Base Mutation (TBM), which may be defined as substitution at adjacent nucleotides (primarily substitutions at two adjacent nucleotides, but substitutions at three adjacent nucleotides have been observed.

The term “variant” refers to a nucleic acid sequence that is different from a nucleic acid reference. Typical nucleic acid sequence variant includes without limitation single nucleotide polymorphism (SNP), short deletion and insertion polymorphisms (Indel), copy number variation (CNV), microsatellite markers or short tandem repeats and structural variation. Somatic variant calling is the effort to identify variants present at low frequency in the DNA sample. Somatic variant calling is of interest in the context of cancer treatment. Cancer is caused by an accumulation of mutations in DNA. A DNA sample from a tumor is generally heterogeneous, including some normal cells, some cells at an early stage of cancer progression (with fewer mutations), and some late-stage cells (with more mutations). Because of this heterogeneity, when sequencing a tumor (e.g., from an FFPE sample), somatic mutations will often appear at a low frequency. For example, a SNV might be seen in only 10% of the reads covering a given base. A variant that is to be classified as somatic or germline by the variant classifier is also referred to herein as the “variant under test”.

The term “noise” refers to a mistaken variant call resulting from one or more errors in the sequencing process and/or in the variant call application.

The term “variant frequency” represents the relative frequency of an allele (variant of a gene) at a particular locus in a population, expressed as a fraction or percentage. For example, the fraction or percentage may be the fraction of all chromosomes in the population that carry that allele. By way of example, sample variant frequency represents the relative frequency of an allele/variant at a particular locus/position along a genomic sequence of interest over a “population” corresponding to the number of reads and/or samples obtained for the genomic sequence of interest from an individual. As another example, a baseline variant frequency represents the relative frequency of an allele/variant at a particular locus/position along one or more baseline genomic sequences where the “population” corresponding to the number of reads and/or samples obtained for the one or more baseline genomic sequences from a population of normal individuals.

The term “variant allele frequency (VAF)” refers to the percentage of sequenced reads observed matching the variant divided by the overall coverage at the target position. VAF is a measure of the proportion of sequenced reads carrying the variant.

The terms “position”, “designated position”, and “locus” refer to a location or coordinate of one or more nucleotides within a sequence of nucleotides. The terms “position”, “designated position”, and “locus” also refer to a location or coordinate of one or more base pairs in a sequence of nucleotides.

The term “haplotype” refers to a combination of alleles at adjacent sites on a chromosome that are inherited together. A haplotype may be one locus, several loci, or an entire chromosome depending on the number of recombination events that have occurred between a given set of loci, if any occurred.

The term “threshold” herein refers to a numeric or non-numeric value that is used as a cutoff to characterize a sample, a nucleic acid, or portion thereof (e.g., a read). A threshold may be varied based upon empirical analysis. The threshold may be compared to a measured or calculated value to determine whether the source giving rise to such value suggests should be classified in a particular manner. Threshold values can be identified empirically or analytically. The choice of a threshold is dependent on the level of confidence that the user wishes to have to make the classification. The threshold may be chosen for a particular purpose (e.g., to balance sensitivity and selectivity). As used herein, the term “threshold” indicates a point at which a course of analysis may be changed and/or a point at which an action may be triggered. A threshold is not required to be a predetermined number. Instead, the threshold may be, for instance, a function that is based on a plurality of factors. The threshold may be adaptive to the circumstances. Moreover, a threshold may indicate an upper limit, a lower limit, or a range between limits.

In some implementations, a metric or score that is based on sequencing data may be compared to the threshold. As used herein, the terms “metric” or “score” may include values or results that were determined from the sequencing data or may include functions that are based on the values or results that were determined from the sequencing data. Like a threshold, the metric or score may be adaptive to the circumstances. For instance, the metric or score may be a normalized value. As an example of a score or metric, one or more implementations may use count scores when analyzing the data. A count score may be based on number of sample reads. The sample reads may have undergone one or more filtering stages such that the sample reads have at least one common characteristic or quality. For example, each of the sample reads that are used to determine a count score may have been aligned with a reference sequence or may be assigned as a potential allele. The number of sample reads having a common characteristic may be counted to determine a read count. Count scores may be based on the read count. In some implementations, the count score may be a value that is equal to the read count. In other implementations, the count score may be based on the read count and other information. For example, a count score may be based on the read count for a particular allele of a genetic locus and a total number of reads for the genetic locus. In some implementations, the count score may be based on the read count and previously-obtained data for the genetic locus. In some implementations, the count scores may be normalized scores between predetermined values. The count score may also be a function of read counts from other loci of a sample or a function of read counts from other samples that were concurrently run with the sample-of-interest. For instance, the count score may be a function of the read count of a particular allele and the read counts of other loci in the sample and/or the read counts from other samples. As one example, the read counts from other loci and/or the read counts from other samples may be used to normalize the count score for the particular allele.

The terms “coverage” or “fragment coverage” refer to a count or other measure of a number of sample reads for the same fragment of a sequence. A read count may represent a count of the number of reads that cover a corresponding fragment. Alternatively, the coverage may be determined by multiplying the read count by a designated factor that is based on historical knowledge, knowledge of the sample, knowledge of the locus, etc.

The term “read depth” (conventionally a number followed by “x”) refers to the number of sequenced reads with overlapping alignment at the target position. This is often expressed as an average or percentage exceeding a cutoff over a set of intervals (such as exons, genes, or panels). For example, a clinical report might say that a panel average coverage is 1,105× with 98% of targeted bases covered > 100×.

The terms “base call quality score” or “Q score” refer to a PHRED-scaled probability ranging from 0-50 inversely proportional to the probability that a single sequenced base is correct. For example, a T base call with Q of 20 is considered likely correct with a probability of 99.99%. Any base call with Q<20 should be considered low quality, and any variant identified where a substantial proportion of sequenced reads supporting the variant are of low quality should be considered potentially false positive.

The terms “variant reads” or “variant read number” refer to the number of sequenced reads supporting the presence of the variant.

Regarding “strandedness” (or DNA strandedness), the genetic message in DNA can be represented as a string of the letters A, G, C, and T. For example, 5′ - AGGACA - 3′. Often, the sequence is written in the direction shown here, i.e., with the 5′ end to the left and the 3′ end to the right. DNA may sometimes occur as single-stranded molecule (as in certain viruses), but normally we find DNA as a double-stranded unit. It has a double helical structure with two antiparallel strands. In this case, the word “antiparallel” means that the two strands run in parallel, but have opposite polarity. The double-stranded DNA is held together by pairing between bases and the pairing is always such that adenine (A) pairs with thymine (T) and cytosine (C) pairs with guanine (G). This pairing is referred to as complementarity, and one strand of DNA is said to be the complement of the other. The double-stranded DNA may thus be represented as two strings, like this: 5′ - AGGACA - 3′ and 3′ - TCCTGT - 5′. Note that the two strands have opposite polarity. Accordingly, the strandedness of the two DNA strands can be referred to as the reference strand and its complement, forward and reverse strands, top and bottom strands, sense and antisense strands, or Watson and Crick strands.

The reads alignment (also called reads mapping) is the process of figuring out where in the genome a sequence is from. Once the alignment is performed, the “mapping quality” or the “mapping quality score (MAPQ)” of a given read quantifies the probability that its position on the genome is correct. The mapping quality is encoded in the phred scale where P is the probability that the alignment is not correct. The probability is calculated as: P = 10 ^{(-MAQ/10)}, where MAPQ is the mapping quality. For example, a mapping quality of 40 = 10 to the power of -4, meaning that there is a 0.01% chance that the read was aligned incorrectly. The mapping quality is therefore associated with several alignment factors, such as the base quality of the read, the complexity of the reference genome, and the paired-end information. Regarding the first, if the base quality of the read is low, it means that the observed sequence might be wrong and thus its alignment is wrong. Regarding the second, the mappability refers to the complexity of the genome. Repeated regions are more difficult to map and reads falling in these regions usually get low mapping quality. In this context, the MAPQ reflects the fact that the reads are not uniquely aligned and that their real origin cannot be determined. Regarding the third, in case of paired-end sequencing data, concordant pairs are more likely to be well aligned. The higher is the mapping quality, the better is the alignment. A read aligned with a good mapping quality usually means that the read sequence was good and was aligned with few mismatches in a high mappability region. The MAPQ value can be used as a quality control of the alignment results. The proportion of reads aligned with an MAPQ higher than 20 is usually for downstream analysis.

As used herein, a “signal” refers to a detectable event such as an emission, preferably light emission, for example, in an image. Thus, in preferred implementations, a signal can represent any detectable light emission that is captured in an image (i.e., a “spot”). Thus, as used herein, “signal” can refer to both an actual emission from an analyte of the specimen, and can refer to a spurious emission that does not correlate to an actual analyte. Thus, a signal could arise from noise and could be later discarded as not representative of an actual analyte of a specimen.

As used herein, the term “clump” refers to a group of signals. In particular implementations, the signals are derived from different analytes. In a preferred implementation, a signal clump is a group of signals that cluster together. In a more preferred implementation, a signal clump represents a physical region covered by one amplified oligonucleotide. Each signal clump should be ideally observed as several signals (one per template cycle, and possibly more due to cross-talk). Accordingly, duplicate signals are detected where two (or more) signals are included in a template from the same clump of signals.

As used herein, terms such as “minimum,” “maximum,” “minimize,” “maximize” and grammatical variants thereof can include values that are not the absolute maxima or minima. In some implementations, the values include near maximum and near minimum values. In other implementations, the values can include local maximum and/or local minimum values. In some implementations, the values include only absolute maximum or minimum values.

As used herein, “cross-talk” refers to the detection of signals in one image that are also detected in a separate image. In a preferred implementation, cross-talk can occur when an emitted signal is detected in two separate detection channels. For example, where an emitted signal occurs in one color, the emission spectrum of that signal may overlap with another emitted signal in another color. In a preferred implementation, fluorescent molecules used to indicate the presence of nucleotide bases A, C, G and T are detected in separate channels. However, because the emission spectra of A and C overlap, some of the C color signal may be detected during detection using the A color channel. Accordingly, cross-talk between the A and C signals allows signals from one color image to appear in the other color image. In some implementations, G and T cross-talk. In some implementations, the amount of cross-talk between channels is asymmetric. It will be appreciated that the amount of cross-talk between channels can be controlled by, among other things, the selection of signal molecules having an appropriate emission spectrum as well as selection of the size and wavelength range of the detection channel.

As used herein, “register”, “registering”, “registration” and like terms refer to any process to correlate signals in an image or data set from a first time point or perspective with signals in an image or data set from another time point or perspective. For example, registration can be used to align signals from a set of images to form a template. In another example, registration can be used to align signals from other images to a template. One signal may be directly or indirectly registered to another signal. For example, a signal from image “S” may be registered to image “G” directly. As another example, a signal from image “N” may be directly registered to image “G”, or alternatively, the signal from image “N” may be registered to image “S”, which has previously been registered to image “G”. Thus, the signal from image “N” is indirectly registered to image “G”.

As used herein, the term “fiducial” is intended to mean a distinguishable point of reference in or on an object. The point of reference can be, for example, a mark, second object, shape, edge, area, irregularity, channel, pit, post or the like. The point of reference can be present in an image of the object or in another data set derived from detecting the object. The point of reference can be specified by an x and/or y coordinate in a plane of the object. Alternatively or additionally, the point of reference can be specified by a z coordinate that is orthogonal to the xy plane, for example, being defined by the relative locations of the object and a detector. One or more coordinates for a point of reference can be specified relative to one or more other analytes of an object or of an image or other data set derived from the object.

As used herein, the term “optical signal” is intended to include, for example, fluorescent, luminescent, scatter, or absorption signals. Optical signals can be detected in the ultraviolet (UV) range (about 200 to 390 nm), visible (VIS) range (about 391 to 770 nm), infrared (IR) range (about 0.771 to 25 microns), or other range of the electromagnetic spectrum. Optical signals can be detected in a way that excludes all or part of one or more of these ranges.

As used herein, the term “signal level” is intended to mean an amount or quantity of detected energy or coded information that has a desired or predefined characteristic. For example, an optical signal can be quantified by one or more of intensity, wavelength, energy, frequency, power, luminance or the like. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

As used herein, the term “simulate” is intended to mean creating a representation or model of a physical thing or action that predicts characteristics of the thing or action. The representation or model can in many cases be distinguishable from the thing or action. For example, the representation or model can be distinguishable from a thing with respect to one or more characteristic such as color, intensity of signals detected from all or part of the thing, size, or shape. In particular implementations, the representation or model can be idealized, exaggerated, muted, or incomplete when compared to the thing or action. Thus, in some implementations, a representation of model can be distinguishable from the thing or action that it represents, for example, with respect to at least one of the characteristics set forth above. The representation or model can be provided in a computer readable format or medium such as one or more of those set forth elsewhere herein.

As used herein, the term “specific signal” is intended to mean detected energy or coded information that is selectively observed over other energy or information such as background energy or information. For example, a specific signal can be an optical signal detected at a particular intensity, wavelength or color; an electrical signal detected at a particular frequency, power or field strength; or other signals known in the art pertaining to spectroscopy and analytical detection.

As used herein, the term “swath” is intended to mean a rectangular portion of an object. The swath can be an elongated strip that is scanned by relative movement between the object and a detector in a direction that is parallel to the longest dimension of the strip. Generally, the width of the rectangular portion or strip will be constant along its full length. Multiple swaths of an object can be parallel to each other. Multiple swaths of an object can be adjacent to each other, overlapping with each other, abutting each other, or separated from each other by an interstitial area.

As used herein, the term “variance” is intended to mean a difference between that which is expected and that which is observed or a difference between two or more observations. For example, variance can be the discrepancy between an expected value and a measured value. Variance can be represented using statistical functions such as standard deviation, the square of standard deviation, coefficient of variation or the like.

As used herein, the term “xy coordinates” is intended to mean information that specifies location, size, shape, and/or orientation in an xy plane. The information can be, for example, numerical coordinates in a Cartesian system. The coordinates can be provided relative to one or both of the x and y axes or can be provided relative to another location in the xy plane. For example, coordinates of an analyte of an object can specify the location of the analyte relative to location of a fiducial or other analyte of the object.

As used herein, the term “xy plane” is intended to mean a 2 dimensional area defined by straight line axes x and y. When used in reference to a detector and an object observed by the detector, the area can be further specified as being orthogonal to the direction of observation between the detector and object being detected.

As used herein, the term “z coordinate” is intended to mean information that specifies the location of a point, line or area along an axis that is orthogonal to an xy plane. In particular implementations, the z axis is orthogonal to an area of an object that is observed by a detector. For example, the direction of focus for an optical system may be specified along the z axis.

In some implementations, acquired signal data is transformed using an affine transformation. In some such implementations, template generation makes use of the fact that the affine transforms between color channels are consistent between runs. Because of this consistency, a set of default offsets can be used when determining the coordinates of the analytes in a specimen. For example, a default offsets file can contain the relative transformation (shift, scale, skew) for the different channels relative to one channel, such as the A channel. In other implementations, however, the offsets between color channels drift during a run and/or between runs, making offset-driven template generation difficult. In such implementations, the methods and systems provided herein can utilize offset-less template generation, which is described further below.

In some implementations of the above implementations, the system can comprise a flow cell. In some implementations, the flow cell comprises lanes, or other configurations, of tiles, wherein at least some of the tiles comprise one or more arrays of analytes. In some implementations, the analytes comprise a plurality of molecules such as nucleic acids. In certain aspects, the flow cell is configured to deliver a labeled nucleotide base to an array of nucleic acids, thereby extending a primer hybridized to a nucleic acid within an analyte so as to produce a signal corresponding to an analyte comprising the nucleic acid. In preferred implementations, the nucleic acids within an analyte are identical or substantially identical to each other.

In some of the systems for image analysis described herein, each image in the set of images includes color signals, wherein a different color corresponds to a different nucleotide base. In some implementations, each image of the set of images comprises signals having a single color selected from at least four different colors. In some implementations, each image in the set of images comprises signals having a single color selected from four different colors. In some of the systems described herein, nucleic acids can be sequenced by providing four different labeled nucleotide bases to the array of molecules so as to produce four different images, each image comprising signals having a single color, wherein the signal color is different for each of the four different images, thereby producing a cycle of four color images that corresponds to the four possible nucleotides present at a particular position in the nucleic acid. In certain aspects, the system comprises a flow cell that is configured to deliver additional labeled nucleotide bases to the array of molecules, thereby producing a plurality of cycles of color images.

In preferred implementations, the methods provided herein can include determining whether a processor is actively acquiring data or whether the processor is in a low activity state. Acquiring and storing large numbers of high-quality images typically requires massive amounts of storage capacity. Additionally, once acquired and stored, the analysis of image data can become resource intensive and can interfere with processing capacity of other functions, such as ongoing acquisition and storage of additional image data. Accordingly, as used herein, the term low activity state refers to the processing capacity of a processor at a given time. In some implementations, a low activity state occurs when a processor is not acquiring and/or storing data. In some implementations, a low activity state occurs when some data acquisition and/or storage is taking place, but additional processing capacity remains such that image analysis can occur at the same time without interfering with other functions.

As used herein, “identifying a conflict” refers to identifying a situation where multiple processes compete for resources. In some such implementations, one process is given priority over another process. In some implementations, a conflict may relate to the need to give priority for allocation of time, processing capacity, storage capacity or any other resource for which priority is given. Thus, in some implementations, where processing time or capacity is to be distributed between two processes such as either analyzing a data set and acquiring and/or storing the data set, a conflict between the two processes exists and can be resolved by giving priority to one of the processes.

Also provided herein are systems for performing image analysis. The systems can include a processor; a storage capacity; and a program for image analysis, the program comprising instructions for processing a first data set for storage and the second data set for analysis, wherein the processing comprises acquiring and/or storing the first data set on the storage device and analyzing the second data set when the processor is not acquiring the first data set. In certain aspects, the program includes instructions for identifying at least one instance of a conflict between acquiring and/or storing the first data set and analyzing the second data set; and resolving the conflict in favor of acquiring and/or storing image data such that acquiring and/or storing the first data set is given priority. In certain aspects, the first data set comprises image files obtained from an optical imaging device. In certain aspects, the system further comprises an optical imaging device. In some implementations, the optical imaging device comprises a light source and a detection device.

As used herein, the term “program” refers to instructions or commands to perform a task or process. The term “program” can be used interchangeably with the term module. In certain implementations, a program can be a compilation of various instructions executed under the same set of commands. In other implementations, a program can refer to a discrete batch or file.

Set forth below are some of the surprising effects of utilizing the methods and systems for performing image analysis set forth herein. In some sequencing implementations, an important measure of a sequencing system’s utility is its overall efficiency. For example, the amount of mappable data produced per day and the total cost of installing and running the instrument are important aspects of an economical sequencing solution. To reduce the time to generate mappable data and to increase the efficiency of the system, real-time base calling can be enabled on an instrument computer and can run in parallel with sequencing chemistry and imaging. This allows much of the data processing and analysis to be completed before the sequencing chemistry finishes. Additionally, it can reduce the storage required for intermediate data and limit the amount of data that needs to travel across the network.

While sequence output has increased, the data per run transferred from the systems provided herein to the network and to secondary analysis processing hardware has substantially decreased. By transforming data on the instrument computer (acquiring computer), network loads are dramatically reduced. Without these on-instrument, off-network data reduction techniques, the image output of a fleet of DNA sequencing instruments would cripple most networks.

The widespread adoption of the high-throughput DNA sequencing instruments has been driven in part by ease of use, support for a range of applications, and suitability for virtually any lab environment. The highly efficient algorithms presented herein allow significant analysis functionality to be added to a simple workstation that can control sequencing instruments. This reduction in the requirements for computational hardware has several practical benefits that will become even more important as sequencing output levels continue to increase. For example, by performing image analysis and base calling on a simple tower, heat production, laboratory footprint, and power consumption are kept to a minimum. In contrast, other commercial sequencing technologies have recently ramped up their computing infrastructure for primary analysis, with up to five times more processing power, leading to commensurate increases in heat output and power consumption. Thus, in some implementations, the computational efficiency of the methods and systems provided herein enables customers to increase their sequencing throughput while keeping server hardware expenses to a minimum.

Accordingly, in some implementations, the methods and/or systems presented herein act as a state machine, keeping track of the individual state of each specimen, and when it detects that a specimen is ready to advance to the next state, it does the appropriate processing and advances the specimen to that state. A more detailed example of how the state machine monitors a file system to determine when a specimen is ready to advance to the next state according to a preferred implementation is set forth in Example 1 below.

In preferred implementations, the methods and systems provided herein are multi-threaded and can work with a configurable number of threads. Thus, for example in the context of nucleic acid sequencing, the methods and systems provided herein are capable of working in the background during a live sequencing run for real-time analysis, or it can be run using a pre-existing set of image data for off-line analysis. In certain preferred implementations, the methods and systems handle multi-threading by giving each thread its own subset of specimen for which it is responsible. This minimizes the possibility of thread contention.

A method of the present disclosure can include a step of obtaining a target image of an object using a detection apparatus, wherein the image includes a repeating pattern of analytes on the object. Detection apparatus that are capable of high resolution imaging of surfaces are particularly useful. In particular implementations, the detection apparatus will have sufficient resolution to distinguish analytes at the densities, pitches, and/or analyte sizes set forth herein. Particularly useful are detection apparatus capable of obtaining images or image data from surfaces. Example detectors are those that are configured to maintain an object and detector in a static relationship while obtaining an area image. Scanning apparatus can also be used. For example, an apparatus that obtains sequential area images (e.g., so called ‘step and shoot’ detectors) can be used. Also useful are devices that continually scan a point or line over the surface of an object to accumulate data to construct an image of the surface. Point scanning detectors can be configured to scan a point (i.e., a small detection area) over the surface of an object via a raster motion in the x-y plane of the surface. Line scanning detectors can be configured to scan a line along the y dimension of the surface of an object, the longest dimension of the line occurring along the x dimension. It will be understood that the detection device, object or both can be moved to achieve scanning detection. Detection apparatus that are particularly useful, for example in nucleic acid sequencing applications, are described in U.S. Pat. App. Pub. Nos. 2012/0270305Al; 2013/0023422 Al; and 2013/0260372 Al; and U.S. Pat. Nos. 5,528,050; 5,719,391; 8,158,926 and 8,241,573, each of which is incorporated herein by reference.

The implementations disclosed herein may be implemented as a method, apparatus, system, or article of manufacture using programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), coarse grained reconfigurable architectures (CGRAs), application-specific integrated circuits (ASICs), complex programmable logic devices (CPLDs), programmable logic arrays (PLAs), microprocessors, or other similar processing devices. In particular implementations, information or algorithms set forth herein are present in non-transient storage media.

In particular implementations, a computer implemented method set forth herein can occur in real time while multiple images of an object are being obtained. Such real time analysis is particularly useful for nucleic acid sequencing applications wherein an array of nucleic acids is subjected to repeated cycles of fluidic and detection steps. Analysis of the sequencing data can often be computationally intensive such that it can be beneficial to perform the methods set forth herein in real time or in the background while other data acquisition or analysis algorithms are in process. Example real time analysis methods that can be used with the present methods are those used for the MiSeq and HiSeq sequencing devices commercially available from Illumina, Inc. (San Diego, Calif.) and/or described in U.S. Pat. App. Pub. No. 2012/0020537 A1, which is incorporated herein by reference.

An example data analysis system, formed by one or more programmed computers, with programming being stored on one or more machine readable media with code executed to carry out one or more steps of methods described herein. In one implementation, for example, the system includes an interface designed to permit networking of the system to one or more detection systems (e.g., optical imaging systems) that are configured to acquire data from target objects. The interface may receive and condition data, where appropriate. In particular implementations the detection system will output digital image data, for example, image data that is representative of individual picture elements or pixels that, together, form an image of an array or other object. A processor processes the received detection data in accordance with a one or more routines defined by processing code. The processing code may be stored in various types of memory circuitry.

In accordance with the presently contemplated implementations, the processing code executed on the detection data includes a data analysis routine designed to analyze the detection data to determine the locations and metadata of individual analytes visible or encoded in the data, as well as locations at which no analyte is detected (i.e., where there is no analyte, or where no meaningful signal was detected from an existing analyte). In particular implementations, analyte locations in an array will typically appear brighter than non-analyte locations due to the presence of fluorescing dyes attached to the imaged analytes. It will be understood that the analytes need not appear brighter than their surrounding area, for example, when a target for the probe at the analyte is not present in an array being detected. The color at which individual analytes appear may be a function of the dye employed as well as of the wavelength of the light used by the imaging system for imaging purposes. Analytes to which targets are not bound or that are otherwise devoid of a particular label can be identified according to other characteristics, such as their expected location in the microarray.

Once the data analysis routine has located individual analytes in the data, a value assignment may be carried out. In general, the value assignment will assign a digital value to each analyte based upon characteristics of the data represented by detector components (e.g., pixels) at the corresponding location. That is, for example when imaging data is processed, the value assignment routine may be designed to recognize that a specific color or wavelength of light was detected at a specific location, as indicated by a group or cluster of pixels at the location. In a typical DNA imaging application, for example, the four common nucleotides will be represented by four separate and distinguishable colors. Each color, then, may be assigned a value corresponding to that nucleotide.

As used herein, the terms “module”, “system,” or “system controller” may include a hardware and/or software system and circuitry that operates to perform one or more functions. For example, a module, system, or system controller may include a computer processor, controller, or other logic-based device that performs operations based on instructions stored on a tangible and non-transitory computer readable storage medium, such as a computer memory. Alternatively, a module, system, or system controller may include a hard-wired device that performs operations based on hard-wired logic and circuitry. The module, system, or system controller shown in the attached figures may represent the hardware and circuitry that operates based on software or hardwired instructions, the software that directs hardware to perform the operations, or a combination thereof. The module, system, or system controller can include or represent hardware circuits or circuitry that include and/or are connected with one or more processors, such as one or computer microprocessors.

As used herein, the terms “software” and “firmware” are interchangeable and include any computer program stored in memory for execution by a computer, including RAM memory, ROM memory, EPROM memory, EEPROM memory, and non-volatile RAM (NVRAM) memory. The above memory types are examples only and are thus not limiting as to the types of memory usable for storage of a computer program.

In the molecular biology field, one of the processes for nucleic acid sequencing in use is sequencing-by-synthesis. The technique can be applied to massively parallel sequencing projects. For example, by using an automated platform, it is possible to carry out hundreds of thousands of sequencing reactions simultaneously. Thus, one of the implementations of the present invention relates to instruments and methods for acquiring, storing, and analyzing image data generated during nucleic acid sequencing.

Enormous gains in the amount of data that can be acquired and stored make streamlined image analysis methods even more beneficial. For example, the image analysis methods described herein permit both designers and end users to make efficient use of existing computer hardware. Accordingly, presented herein are methods and systems which reduce the computational burden of processing data in the face of rapidly increasing data output. For example, in the field of DNA sequencing, yields have scaled 15-fold over the course of a recent year and can now reach hundreds of gigabases in a single run of a DNA sequencing device. If computational infrastructure requirements grew proportionately, large genome-scale experiments would remain out of reach to most researchers. Thus, the generation of more raw sequence data will increase the need for secondary analysis and data storage, making optimization of data transport and storage extremely valuable. Some implementations of the methods and systems presented herein can reduce the time, hardware, networking, and laboratory infrastructure requirements needed to produce usable sequence data.

The present disclosure describes various methods and systems for carrying out the methods. Examples of some of the methods are described as a series of steps. However, it should be understood that implementations are not limited to the particular steps and/or order of steps described herein. Steps may be omitted, steps may be modified, and/or other steps may be added. Moreover, steps described herein may be combined, steps may be performed simultaneously, steps may be performed concurrently, steps may be split into multiple sub-steps, steps may be performed in a different order, or steps (or a series of steps) may be re-performed in an iterative fashion. In addition, although different methods are set forth herein, it should be understood that the different methods (or steps of the different methods) may be combined in other implementations.

In some implementations, a processing unit, processor, module, or computing system that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

Moreover, the operations of the methods described herein can be sufficiently complex such that the operations cannot be mentally performed by an average human being or a person of ordinary skill in the art within a commercially reasonable time period. For example, the methods may rely on relatively complex computations such that such a person cannot complete the methods within a commercially reasonable time.

Throughout this application various publications, patents or patent applications have been referenced. The disclosures of these publications in their entireties are hereby incorporated by reference in this application in order to more fully describe the state of the art to which this invention pertains.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

Although the invention has been described with reference to the examples provided above, it should be understood that various modifications can be made without departing from the invention.

The modules in this application can be implemented in hardware or software and need not be divided up in precisely the same blocks as shown in the figures. Some can also be implemented on different processors or computers or spread among a number of different processors or computers. In addition, it will be appreciated that some of the modules can be combined, operated in parallel or in a different sequence than that shown in the figures without affecting the functions achieved. Also as used herein, the term “module” can include “sub-modules”, which themselves can be considered herein to constitute modules. The blocks in the figures designated as modules can also be thought of as flowchart steps in a method.

As used herein, the “identification” of an item of information does not necessarily require the direct specification of that item of information. Information can be “identified” in a field by simply referring to the actual information through one or more layers of indirection, or by identifying one or more items of different information which are together sufficient to determine the actual item of information. In addition, the term “specify” is used herein to mean the same as “identify”.

As used herein, a given signal, event or value is “in dependence upon” a predecessor signal, event or value of the predecessor signal, event or value influenced by the given signal, event, or value. If there is an intervening processing element, step or time period, the given signal, event, or value can still be “in dependence upon” the predecessor signal, event, or value. If the intervening processing element or step combines more than one signal, event or value, the signal output of the processing element or step is considered “in dependence upon” each of the signal, event, or value inputs. If the given signal, event, or value is the same as the predecessor signal, event, or value, this is merely a degenerate case in which the given signal, event or value is still considered to be “in dependence upon” or “dependent on” or “based on” the predecessor signal, event, or value. “Responsiveness” of a given signal, event or value upon another signal, event or value is defined similarly.

As used herein, “concurrently” or “in parallel” does not require exact simultaneity. It is sufficient if the processing of one of the images begins before the processing of another of the images completes. It is sufficient if the outputting of one of the base calls begins before the outputting of another of the base calls completes.

This application refers to “sequencing images,” “cluster images” and “cluster intensity images” interchangeably.

### Clauses

The technology disclosed, in particularly, the clauses disclosed in this section, can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections - these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

1. A computer-implemented method of base calling, including:
accessing a time series sequence of a read, wherein respective time series elements in the time series sequence represent respective bases in the read;
generating a composite sequence for the read based on respective aggregate transformations of time series elements in the time series sequence, wherein a subject composite element in the composite sequence is generated based on an aggregate transformation of a corresponding group of time series elements in the time series sequence; and
processing the composite sequence as an aggregate and generating a base call sequence having respective base calls for the respective bases in the read.

2. The computer-implemented method of clause 1, further including generating the composite sequence for the read based on the respective aggregate transformations of respective sliding windows of the time series elements in the time series sequence.

3. The computer-implemented method of clause 2, wherein the respective sliding windows have overlapping time series elements.

4. The computer-implemented method of clause 2, wherein the respective sliding windows are nonoverlapping.

5. The computer-implemented method of clause 2, wherein each of the respective sliding windows has N time series elements, where N is an integer greater than 1.

6. The computer-implemented method of clause 1, wherein the respective base calls for the respective bases in the read are concurrently generated.

7. The computer-implemented method of clause 1, wherein a linear projection layer is trained to learn weights that apply the respective aggregate transformations and generate the composite sequence.

8. The computer-implemented method of clause 7, wherein the linear projection layer is trained to learn the weights that apply the respective aggregate transformations on the respective sliding windows of the time series elements in the time series sequence and generate the composite sequence.

9. The computer-implemented method of clause 1, wherein a multi-headed attention encoder is trained to process the composite sequence as the aggregate and to generate an alternative representation of the composite sequence.

10. The computer-implemented method of clause 9, wherein the multi-headed attention encoder is trained using self-attention.

11. The computer-implemented method of clause 9, wherein the multi-headed attention encoder is trained using cross-attention.

12. The computer-implemented method of clause 9, wherein an output layer is trained to process the alternative representation of the composite sequence and generate the base call sequence.

13. The computer-implemented method of clause 12, wherein the output layer is trained to concurrently generate base-wise classification likelihoods for each composite element in the composite sequence.

14. The computer-implemented method of clause 13, wherein a base call for a subject base in the read is determined based on a maximum base-wise classification likelihood generated by the output layer for a corresponding composite element in the composite sequence.

15. The computer-implemented method of clause 9, wherein the multi-headed attention encoder is trained to correct for systematic errors in cluster amplification that are encoded in the read.

16. The computer-implemented method of clause 15, wherein the systematic errors include phasing and prephasing errors.

17. The computer-implemented method of clause 15, wherein the systematic errors include context dependent intensity modulations.

18. The computer-implemented method of clause 9, wherein the multi-headed attention encoder is trained to analyze backward and forward flanking composite elements in conjunction with analyzing a subject composite element in the composite sequence.

19. The computer-implemented method of clause 18, wherein a forward mask of the multi-headed attention encoder is deactivated to account for the forward flanking composite elements.

20. The computer-implemented method of clause 9, wherein the multi-headed attention encoder is trained on read data from multiple human gene sources.

21. The computer-implemented method of clause 20, wherein the trained multi-headed attention encoder is tested on read data from multiple bacteria gene sources.

22. The computer-implemented method of clause 1, wherein the time series sequence has a dimensionality of L × C, where L is a number of bases in the read, and C is a number of channels.

23. The computer-implemented method of clause 22, wherein the composite sequence has a dimensionality of (L-(W-1) × (W × C), where W is a size of the respective sliding windows.

24. The computer-implemented method of clause 23, wherein the base call sequence has a dimensionality of (L-(W-1) × 4.

25. The computer-implemented method of clause 22, wherein the composite sequence has a dimensionality of L × C in dependence upon zero padded composite elements.

26. The computer-implemented method of clause 25, wherein the base call sequence has a dimensionality of L × C.

27. The computer-implemented method of clause 1, wherein the respective time series elements are respective intensity values for respective sequencing cycles of a sequencing run.

28. The computer-implemented method of clause 27, wherein each of the respective intensity values has respective channel-specific measurements for respective channels.

29. The computer-implemented method of clause 28, wherein the respective intensity values are corrected for scale variation and shift variation.

30. The computer-implemented method of clause 1, wherein the respective time series elements are respective voltage values for respective sequencing cycles of a sequencing run.

31. The computer-implemented method of clause 1, wherein the respective time series elements are respective current values for respective sequencing cycles of a sequencing run.

32. The computer-implemented method of clause 1, wherein the respective time series elements are supplemented with respective state values for respective sequencing cycles of a sequencing run.

33. The computer-implemented method of clause 32, wherein the respective state values are channel-specific.

34. The computer-implemented method of clause 1, wherein the multi-headed attention encoder uses a positional embedding to determine relative inter-element spatial arrangement of composite elements in the composite sequence.

35. The computer-implemented method of clause 34, wherein the positional embedding is learned during training of the multi-headed attention encoder.

36. The computer-implemented method of clause 34, wherein the positional embedding is provided as a Fourier embedding.

37. A computer-implemented method of base calling, including:
accessing a time series sequence of a read, wherein respective time series elements in the time series sequence represent respective bases in the read;
generating a composite sequence for the read based on respective aggregate transformations of respective sliding windows of time series elements in the time series sequence, wherein a subject composite element in the composite sequence is generated based on an aggregate transformation of a corresponding window of time series elements in the time series sequence; and
processing the composite sequence as an aggregate and generating a base call sequence having respective base calls for the respective bases in the read.

38. A computer-implemented method of base calling, including:

processing sequencing data through a Transformer-based sequence-to-sequence base caller, and generating one or more base calls as output.

What is claimed is:

1. A system comprising:
at least one processor; and
a non-transitory computer readable medium comprising instructions that, when executed by the at least one processor, cause the system to:
access a time series sequence of a read, wherein respective time series elements in the time series sequence represent respective bases in the read;
generate a composite sequence for the read based on respective aggregate transformations of time series elements in the time series sequence, wherein a subject composite element in the composite sequence is generated based on an aggregate transformation of a corresponding group of time series elements in the time series sequence; and
process the composite sequence as an aggregate and generating a base call sequence having respective base calls for the respective bases in the read.

2. The system of claim 1, further comprising instructions that, when executed by the at least one processor, cause the system to generate the composite sequence for the read based on the respective aggregate transformations of respective sliding windows of the time series elements in the time series sequence.

3. The system of claim 2, wherein the respective sliding windows have overlapping time series elements.

4. The system of claim 2, wherein the respective sliding windows are non-overlapping.

5. The system of claim 1, further comprising instructions that, when executed by the at least one processor, cause the system to concurrently generate the respective base calls for the respective bases in the read.

6. The system of claim 1, wherein a linear projection layer is trained to learn weights that apply the respective aggregate transformations and generate the composite sequence.

7. The system of claim 6, wherein the linear projection layer is trained to learn the weights that apply the respective aggregate transformations on respective sliding windows of the time series elements in the time series sequence and generate the composite sequence.

8. A non-transitory computer readable medium comprising instructions that, when executed by at least one processor, cause a system to:
access a time series sequence of a read, wherein respective time series elements in the time series sequence represent respective bases in the read;
generate a composite sequence for the read based on respective aggregate transformations of time series elements in the time series sequence, wherein a subject composite element in the composite sequence is generated based on an aggregate transformation of a corresponding group of time series elements in the time series sequence; and
process the composite sequence as an aggregate and generating a base call sequence having respective base calls for the respective bases in the read.

9. The non-transitory computer readable medium of claim 8, wherein a multi-headed attention encoder is trained to process the composite sequence as the aggregate and to generate an alternative representation of the composite sequence.

10. The non-transitory computer readable medium of claim 9, wherein an output layer is trained to process the alternative representation of the composite sequence and generate the base call sequence.

11. The non-transitory computer readable medium of claim 10, wherein the output layer is trained to concurrently generate base-wise classification likelihoods for each composite element in the composite sequence.

12. The non-transitory computer readable medium of claim 11, wherein a base call for a subject base in the read is determined based on a maximum base-wise classification likelihood generated by the output layer for a corresponding composite element in the composite sequence.

13. The non-transitory computer readable medium of claim 9, wherein the multi-headed attention encoder is trained to correct for systematic errors in cluster amplification that are encoded in the read.

14. The non-transitory computer readable medium of claim 13, wherein the systematic errors include phasing and prephasing errors.

15. The non-transitory computer readable medium of claim 14, wherein the systematic errors include context dependent intensity modulations.

16. A computer-implemented method of base calling, including:
accessing a time series sequence of a read, wherein respective time series elements in the time series sequence represent respective bases in the read;
generating a composite sequence for the read based on respective aggregate transformations of time series elements in the time series sequence, wherein a subject composite element in the composite sequence is generated based on an aggregate transformation of a corresponding group of time series elements in the time series sequence; and
processing the composite sequence as an aggregate and generating a base call sequence having respective base calls for the respective bases in the read.

17. The computer-implemented method of claim 16, wherein a multi-headed attention encoder is trained to process the composite sequence as the aggregate and to generate an alternative representation of the composite sequence.

18. The computer-implemented method of claim 17, wherein the multi-headed attention encoder is trained to analyze backward and forward flanking composite elements in conjunction with analyzing a subject composite element in the composite sequence.

19. The computer-implemented method of claim 18, wherein a forward mask of the multi-headed attention encoder is deactivated to account for the forward flanking composite elements.

20. The computer-implemented method of claim 16, wherein the respective time series elements are respective intensity values for respective sequencing cycles of a sequencing run.
